(19) United States
(12) Patent Application Publication
(10) Pub. No.: US 2007/0270312 A1
(43) Pub. Date: Nov. 22, 2007

(54) Heteroaroyl-Substituted Serineamides

(76) Inventors: Matthias Witschel, Bad Durkheim (DE); Frank Stelzer, Mannheim (DE); Toralf Kuhn, Mannheim (DE); Liliana Parra Rapado, Offenburg (DE); Michael Rack, Heidelberg (DE); Eike Hupe, Ludwigshafen (DE); Cyrill Zagar, Mannheim (DE); Robert Reinhard, Ludwigshafen (DE); Bernd Sievernich, Hassloch (DE); Thomas Ehrhardt, Speyer (DE)

Correspondence Address: ROTHWELL, FIGG, ERNST & MANBECK, P.C., 1425 K STREET, N.W., SUITE 800, WASHINGTON, DC, 20005, US

(21) Appl. No.: 11/662,586

(22) PCT Filed: Sep. 14, 2005
(86) PCT No.: PCT/EP05/09856

§ 371 Date: Mar. 13, 2007

(30) Foreign Application Priority Data
Sep. 16, 2004 (DE) 10 2004 045 298.9

(51) Int. Cl. A01N 43/10; A01N 43/42; A01N 43/48; A01N 43/56; A01P 13/00; C07D 215/38; C07D 231/00; C07D 333/22; C07D 401/00

(52) U.S. Cl. 504/247; 504/253; 504/280; 504/289; 546/175; 546/275.4; 548/374.1; 549/72

(57) ABSTRACT

The present invention relates to heteroaroyl-substituted serinamides of the formula I 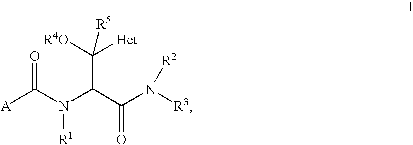
in which the variables A, Het and R¹ to R⁵ are as defined in the description, and to their agriculturally useful salts, to processes and intermediates for their preparation, and to the use of these compounds or of the compositions comprising these compounds for controlling unwanted plants.

The present invention relates to heteroaroyl-substituted serineamides of the formula I 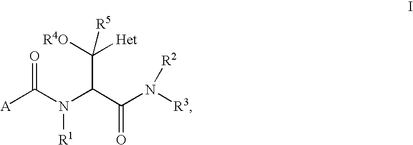
in which the variables are as defined below:
A is 5- or 6-membered heteroaryl having one to four nitrogen atoms or one to three nitrogen atoms and one oxygen or sulfur atom or one oxygen or sulfur atom, which heteroaryl may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
Het is mono- or bicyclic heteroaryl having 5 to 10 ring members comprising 1 to 4 heteroatoms from the group consisting of nitrogen, oxygen and sulfur, which heteroaryl may be partially or fully halogenated and/or may carry 1 to 3 radicals from the group consisting of cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxy-carbonyl-C₁-C₆-alkoxy, C₁-C₆-alkoxycarbonyl-C₁-C₆-alkoxy, amino, C₁-C₆-alkylamino, di-(C₁-C₆-alkyl)amino, C₁-C₆-alkylsulfonylamino, C₁-C₆-haloalkyl-sulfonylamino, aminocarbonylamino, (C₁-C₆-alkylamino)carbonylamino, di-(C₁-C₆-alkyl)aminocarbonylamino, aryl and aryl-(C₁-C₆-alkyl);
R¹, R² are hydrogen, hydroxyl or C₁-C₆-alkoxy;
R³ is C₁-C₆-alkyl, C₁-C₄-cyanoalkyl or C₁-C₆-haloalkyl;
R⁴ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkyl-carbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, C₁-C₆-alkylaminocarbonyl, C₃-C₆-alkenylaminocarbonyl, C₃-C₆-alkynylaminocarbonyl, C₁-C₆-alkylsulfonyl-aminocarbonyl, di-(C₁-C₆-alkyl)aminocarbonyl, N—(C₃-C₆-alkenyl)-N—(C₁-C₆-alkyl)-aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)-aminocarbonyl, N—(C₃-C₆-alkynyl)-N—(C₁-C₆-alkoxy)aminocarbonyl, di-(C₁-C₆-alkyl)aminothiocarbonyl, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-(di-C₁-C₆-alkylamino)imino-C₁-C₆-alkyl or tri-C₁-C₄-alkylsilyl,
   where the alkyl, cycloalkyl and alkoxy radicals mentioned may be partially or fully halogenated and/or may carry one to three of the following groups: cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di-(C₁-C₄-alkyl)-amino, C₁-C₄-alkyl-C₁-C₄-alkoxycarbonylamino, C₁-C₄-alkylcarbonyl, hydroxycarbonyl, C₁-C₄-alkoxycarbonyl, aminocarbonyl, C₁-C₄-alkylamino-carbonyl, di-(C₁-C₄-alkyl)aminocarbonyl or C₁-C₄-alkylcarbonyloxy;
phenyl, phenyl-C₁-C₆-alkyl, phenylcarbonyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl, heterocyclyl, heterocyclyl-C₁-C₆-alkyl, heterocyclylcarbonyl, heterocyclylcarbonyl-C₁-C₆-alkyl, heterocyclyloxycarbonyl, heterocyclylaminocarbonyl, heterocyclylsulfonyl-aminocarbonyl, N—(C₁-C₆-alkyl)-N-(heterocyclyl)aminocarbonyl, or heterocyclyl-C₁-C₆-alkylcarbonyl,
   where the phenyl and the heterocyclyl radical of the 17 last-mentioned substituents may be partially or fully halogenated and/or may carry 1 to 3 of the following groups: nitro, cyano, C₁-C₄-alkyl, C₁-C₄-haloalkyl, C₁-C₄-alkoxy or C₁-C₄-haloalkoxy; or
SO₂R⁶;
R⁵ is hydrogen or C₁-C₆-alkyl;
R⁶ is C₁-C₆-alkyl, C₁-C₆-haloalkyl or phenyl, where the phenyl radical may be partially or fully halogenated and/or may carry one to three of the following groups: C₁-C₆-alkyl, C₁-C₆-haloalkyl or C₁-C₆-alkoxy;
   and their agriculturally useful salts.

Moreover, the invention relates to processes and intermediates for preparing compounds of the formula I, to compositions comprising them and to the use of these derivatives or of the compositions comprising them for controlling harmful plants.

Benzoyl-substituted serineamides having pharmaceutical activity which carry a tetrazolyl radical in the β-position are described, inter alia, in JP 03/294253.

Also known from the literature, for example from WO 03/066576, are herbicidally active phenylalanine derivatives which are unsubstituted in the β-position or may carry unsubstituted or halogen-substituted alkyl, alkenyl or alkynyl radicals.

However, the herbicidal properties of the prior-art compounds and/or their compatibility with crop plants are not entirely satisfactory. Accordingly, it is an object of the present invention to provide novel, in particular herbicidally active, compounds having improved properties.

We have found that this object is achieved by the heteroaroyl-substituted serineamides of the formula I and their herbicidal action.

Furthermore, we have found herbicidal compositions which comprise the compounds I and have very good herbicidal action. Moreover, we have found processes for preparing these compositions and methods for controlling unwanted vegetation using the compounds I.

Depending on the substitution pattern, the compounds of the formula I comprise two or more centers of chiralty, in which case they are present as enantiomers or diastereomer mixtures. The invention provides both the pure enantiomers or diastereomers and their mixtures.

The compounds of the formula I may also be present in the form of their agriculturally useful salts, the nature of the salt generally being immaterial. Suitable salts are, in general, the cations or the acid addition salts of those acids whose cation and anions, respectively, have no adverse effect on the herbicidal action of the compounds I.

Suitable cations are in particular the ions of the alkali metals, preferably lithium, sodium and potassium, of the alkaline earth metals, preferably calcium and magnesium, and of the transition metals, preferably manganese, copper, zinc and iron, and also ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-yl-ammonium, di-(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄alkyl)sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and the anions of C₁-C₄-alkanoic acids, preferably formate, acetate, propionate and butyrate.

The organic moieties mentioned for the substituents R¹-R⁶ or as radicals on phenyl, aryl, heteroaryl or heterocyclyl rings are collective terms for individual enumerations of the specific group members. All hydrocarbon chains, i.e. all alkyl, alkylsilyl, alkenyl, alkynyl, cyanoalkyl, haloalkyl, haloalkenyl, haloalkynyl, alkoxy, haloalkoxy, alkoxyalkyl, alkoxyalkoxyalkyl, alkylcarbonyl, alkenylcarbonyl, alkynylcarbonyl, alkoxycarbonyl, alkenyloxycarbonyl, alkynyloxycarbonyl, alkylamino, alkylsulfonylamino, haloalkyl-sulfonylamino, alkylalkoxycarbonylamino, alkylaminocarbonyl, alkenylaminocarbonyl, alkynylaminocarbonyl, alkylsulfonylaminocarbonyl, dialkylaminocarbonyl, N-alkenyl-N-alkylaminocarbonyl, N-alkynyl-N-alkylamino-carbonyl, N-alkoxy-N-alkylamino-carbonyl, N-alkenyl-N-alkoxyaminocarbonyl, N-alkynyl-N-alkoxyaminocarbonyl, dialkylaminothiocarbonyl, alkylcarbonylalkyl, alkoximinoalkyl, N-(alkylamino)aminoalkyl, N-(dialkylamino)aminoalkyl, phenylalkyl, phenylcarbonylalkyl, N-alkyl-N-phenyl-aminocarbonyl, phenylalkylcarbonyl, arylalkyl, heterocyclylalkyl, heterocyclylcarbonyl-alkyl, N-alkyl-N-heterocyclylaminocarbonyl, heterocyclylalkylcarbonyl, alkylthio and alkylcarbonyloxy moieties may be straight-chain or branched.

Unless indicated otherwise, halogenated substituents preferably carry one to five identical or different halogen atoms. The term halogen denotes in each case fluorine, chlorine, bromine or iodine.

Examples of other meanings are:
C₁-C₄-alkyl and the alkyl moieties of tri-C₁-C₄-alkylsilyl, C₁-C₄-alkylcarbonyloxy, C₁-C₄-alkyl-C₁-C₄-alkoxycarbonylamino, C₁-C₆-alkyliminooxy-C₁-C₄-alkyl, C₁-C₄-alkoxy-C₁-C₄-alkoxy-C₁-C₄-alkyl and aryl(C₁-C₄-alkyl): for example methyl, ethyl, n-propyl, 1-methylethyl, n-butyl, 1-methylpropyl, 2-methylpropyl and 1,1-dimethyl-ethyl;
C₁-C₆-alkyl and the alkyl moieties of C₁-C₆-alkylsulfonylamino, C₁-C₆-alkylsulfonyl-aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, C₁-C₆-alkylcarbonyl-C₁-C₆-alkyl, C₁-C₆-alkoxyimino-C₁-C₆-alkyl, N—(C₁-C₆-alkyl-amino)imino-C₁-C₆-alkyl, N-(di-C₁-C₆-alkylamino)imino-C₁-C₆-alkyl, phenyl-C₁-C₆-alkyl, aryl-(C₁-C₆-alkyl), phenylcarbonyl-C₁-C₆-alkyl, N—(C₁-C₆-alkyl)-N-(phenyl)-aminocarbonyl, heterocyclyl-C₁-C₆-alkyl, hetrocyclylcarbonyl-C₁-C₆-alkyl and N—(C₁-C₆-alkyl)-N-(heterocyclyl)aminocarbonyl:
C₁-C₄-alkyl as mentioned above, and also, for example, n-pentyl, 1-methyl-butyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methyl-pentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethyl-butyl, 2-ethylbutyl, 1,1,2-trimethylpropyl, 1-ethyl-1-methylpropyl and 1-ethyl-3-methylpropyl;
C₁-C₄-alkylcarbonyl: for example methylcarbonyl, ethylcarbonyl, propylcarbonyl, 1-methylethylcarbonyl, butylcarbonyl, 1-methylpropylcarbonyl, 2-methylpropyl-carbonyl or 1,1-dimethylethylcarbonyl;
C₁-C₆-alkylcarbonyl, and the alkylcarbonyl radicals of C₁-C₆-alkylcarbonyl-C₁-C₆-alkyl, phenyl-C₁-C₆-alkylcarbonyl and heterocyclyl-C₁-C₆-alkylcarbonyl: C₁-C₄-alkylcarbonyl as mentioned above, and also, for example, pentylcarbonyl, 1-methyl-butylcarbonyl, 2-methylbutylcarbonyl, 3-methylbutylcarbonyl, 2,2-dimethylpropyl-carbonyl, 1-ethylpropylcarbonyl, hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl;
C₃-C₆-cycloalkyl and the cycloalkyl moieties of C₃-C₆-cycloalkylcarbonyl: monocyclic saturated hydrocarbons having 3 to 6 ring members, such as cyclopropyl, cyclobutyl, cyclopentyl and cyclohexyl;
C₃-C₆-alkenyl and the alkenyl moieties of C₃-C₆-alkenyloxycarbonyl, C₃-C₆-alkenyl-aminocarbonyl, N—(C₃-C₆-alkenyl)-N—(C₁-C₆-alkyl)aminocarbonyl and N—(C₃-C₆-alkenyl)-N—(C₁-C₆-alkoxy)aminocarbonyl: for example 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-ethyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
C₂-C₆-alkenyl and the alkenyl moieties of C₂-C₆-alkenylcarbonyl: C₃-C₆-alkenyl as mentioned above, and also ethenyl;
C₃-C₆-alkynyl and the alkynyl moieties of C₃-C₆-alkynyloxycarbonyl, C₃-C₆-alkynyl-aminocarbonyl, N—(C₃-C₆-alkynyl)-N—(C₁-C₆-alkyl)aminocarbonyl, N—(C₃-C₆-alkynyl)-N—(C₁-C₆-alkoxy)aminocarbonyl: for example 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
C₂-C₆-alkynyl and the alkynyl moieties of C₂-C₆-alkynylcarbonyl: C₃-C₆-alkynyl as mentioned above, and also ethynyl;
C₁-C₄-cyanoalkyl: for example cyanomethyl, 1-cyanoeth-1-yl, 2-cyanoeth-1-yl, 1-cyanoprop-1-yl, 2-cyanoprop-1-yl, 3-cyanoprop-1-yl, 1-cyanoprop-2-yl, 2-cyano-prop-2-yl, 1-cyanobut-1-yl, 2-cyanobut-1-yl, 3-cyanobut-1-yl, 4-cyanobut-1-yl, 1-cyanobut-2-yl, 2-cyanobut-2-yl, 1-cyanobut-3-yl, 2-cyanobut-3-yl, 1-cyano-2-methylprop-3-yl, 2-cyano-2-methylprop-3-yl, 3-cyano-2-methylprop-3-yl and 2-cyanomethylprop-2-yl;
C₁-C₄-haloalkyl: a C₁-C₄-alkyl radical as mentioned above which is partially or fully substituted by fluorine, chlorine, bromine and/or iodine, i.e., for example, chloro-methyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloro-propyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl;
C₁-C₆-haloalkyl and the haloalkyl moieties of C₁-C₆-haloalkylsulfonylamino: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and tridecafluorohexyl;
C₃-C₆-haloalkenyl: a C₃-C₆-alkenyl radical as mentioned above which is partially or fully substituted by fluorine, chlorine, bromine and/or iodine, for example 2-chloro-prop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl;
C₃-C₆-haloalkynyl: a C₃-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₁-C₄-alkoxy and also all the alkoxy moieties of hydroxycarbonyl-C₁-C₄-alkoxy, C₁-C₄-alkoxycarbonyl-C₁-C₄-alkoxy, C₁-C₄-alkoxy-C₁-C₄-alkoxy-C₁-C₄-alkyl and C₁-C₄-alkyl-C₁-C₄-alkoxycarbonylamino: for example methoxy, ethoxy, propoxy, 1-methylethoxy, butoxy, 1-methylpropoxy, 2-methylpropoxy and 1,1-dimethylethoxy;
C₁-C₆-alkoxy and the alkoxy moieties of hydroxycarbon-C₁-C₆-alkoxy, C₁-C₆-alkoxycarbonyl-C₁-C₆-alkoxy, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl and C₁-C₆-alkoxyimino-C₁-C₆-alkyl: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methyl-propoxy and 1-ethyl-2-methylpropoxy;
C₁-C₄-haloalkoxy: a C₁-C₄-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromo-difluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoro-ethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-penta-fluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy and nonafluorobutoxy;
C₁-C₆-haloalkoxy: C₁-C₄-haloalkoxy as mentioned above, and also, for example, 5-fluoropentoxy, 5-chloropentoxy, 5-bromopentoxy, 5-iodopentoxy, undecafluoro-pentoxy, 6-fluorohexoxy, 6-chlorohexoxy, 6-bromohexoxy, 6-iodohexoxy and tridecafluorohexoxy;
C₁-C₆-alkoxy-C₁-C₄-alkyl: C₁-C₄-alkyl which is substituted by C₁-C₆-alkoxy as mentioned above, i.e., for example, methoxymethyl, ethoxymethyl, propoxymethyl, (1-methylethoxy)methyl, butoxymethyl, (1-methylpropoxy)methyl, (2-methyl-propoxy)methyl, (1,1-dimethylethoxy)methyl, 2-(methoxy)ethyl, 2-(ethoxy)ethyl, 2-(propoxy)ethyl, 2-(1-methylethoxy)ethyl, 2-(butoxy)ethyl, 2-(1-methylpropoxy)-ethyl, 2-(2-methylpropoxy)ethyl, 2-(1,1-dimethylethoxy)ethyl, 2-(methoxy)propyl, 2-(ethoxy)propyl, 2-(propoxy)propyl, 2-(1-methylethoxy)propyl, 2-(butoxy)propyl, 2-(1-methylpropoxy)propyl, 2-(2-methylpropoxy)propyl, 2-(1,1-dimethylethoxy)-propyl, 3-(methoxy)propyl, 3-(ethoxy)propyl, 3-(propoxy)propyl, 3-(1-methylethoxy)-propyl, 3-(butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(propoxy)butyl, 2-(1-methylethoxy)butyl, 2-(butoxy)butyl, 2-(1-methylpropoxy)butyl, 2-(2-methyl-propoxy)butyl, 2-(1,1-dimethylethoxy)butyl, 3-(methoxy)butyl, 3-(ethoxy)butyl, 3-(propoxy)butyl, 3-(1-methylethoxy)butyl, 3-(butoxy)butyl, 3-(1-methylpropoxy)-butyl, 3-(2-methylpropoxy)butyl, 3-(1,1-dimethylethoxy)butyl, 4-(methoxy)butyl, 4-(ethoxy)butyl, 4-(propoxy)butyl, 4-(1-methylethoxy)butyl, 4-(butoxy)butyl, 4-(1-methylpropoxy)butyl, 4-(2-methylpropoxy)butyl and 4-(1,1-dimethylethoxy)butyl;
C₁-C₄-alkoxycarbonyl and the alkoxycarbonyl moieties of C₁-C₄-alkoxy-C₁-C₄-alkoxycarbonyl: for example methoxycarbonyl, ethoxycarbonyl, propoxycarbonyl, 1-methylethoxycarbonyl, butoxycarbonyl, 1-methylpropoxycarbonyl, 2-methylpropoxycarbonyl or 1,1-dimethylethoxycarbonyl;
C₁-C₆-alkoxycarbonyl and the alkoxycarbonyl moieties of C₁-C₆-alkoxycarbonyl-C₁-C₆-alkoxy: C₁-C₄-alkoxycarbonyl as mentioned above, and also, for example, pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxy-carbonyl, 3,3-dimethylbutoxycarbonyl, 1-ethylbutoxycarbonyl, 2-ethylbutoxy-carbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl;
C₁-C₄-alkylthio: for example methylthio, ethylthio, propylthio, 1-methylethylthio, butylthio, 1-methylpropylthio, 2-methylpropylthio and 1,1-dimethylethylthio;
C₁-C₆-alkylamino and the alkylamino radicals of N—(C₁-C₆-alkylamino)imino-C₁-C₆-alkyl: for example methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methyl-pentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropyl-amino, 1,2,2-trimethylpropylamino, 1-ethyl-1-methylpropylamino or 1-ethyl-2-methyl-propylamino;
di-(C₁-C₄-alkyl)amino: for example N,N-dimethylamino, N,N-diethylamino, N,N-dipropylamino, N,N-di-(1-methylethyl)amino, N,N-dibutylamino, N,N-di-(1-methylpropyl)amino, N,N-di-(2-methylpropyl)amino, N,N-di-(1,1-dimethyl-ethyl)amino, N-ethyl-N-methylamino, N-methyl-N-propylamino, N-methyl-N-(1-methylethyl)amino, N-butyl-N-methylamino, N-methyl-N-(1-methylpropyl)-amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propyl-amino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methyl-ethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methyl-propyl)-N-(2-methylpropyl)amino, N-(1,1-dimethylethyl)-N-(1-methylpropyl)amino and N-(1,1-dimethylethyl)-N-(2-methylpropyl)amino;
di-(C₁-C₆-alkyl)amino and the dialkylamino radicals of N-(di-C₁-C₆-alkylamino)imino-C₁-C₆-alkyl: di-(C₁-C₄-alkyl)amino as mentioned above, and also, for example, N,N-dipentylamino, N,N-dihexylamino, N-methyl-N-pentylamino, N-ethyl-N-pentyl-amino, N-methyl-N-hexylamino and N-ethyl-N-hexylamino;
(C₁-C₄-alkylamino)carbonyl and the (C₁-C₄-alkylamino)carbonyl moieties of (C₁-C₄-alkylamino)carbonylamino: for example methylaminocarbonyl, ethylaminocarbonyl, propylaminocarbonyl, 1-methylethylaminocarbonyl, butylaminocarbonyl, 1-methyl-propylaminocarbonyl, 2-methylproylaminocarbonyl or 1,1-dimethylaminocarbonyl;
di-(C₁-C₄-alkyl)aminocarbonyl and also di-(C₁-C₄-alkyl)aminocarbonyl moieties of di-(C₁-C₄-alkyl)aminocarbonylamino: for example N,N-dimethylaminocarbonyl, N,N-diethylaminocarbonyl, N,N-di-(1-methylethyl)aminocarbonyl, N,N-dipropyl-aminocarbonyl, N,N-dibutylaminocarbonyl, N,N-di-(1-methylpropyl)aminocarbonyl, N,N-di-(2-methylpropyl)aminocarbonyl, N,N-di-(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylamino-carbonyl, N-butyl-N-propylaminocarbonyl, N-(1-methylpropyl)-N-propylamino-carbonyl, N-(2-methylpropyl)-N-propylaminocarbonyl, N-(1,1-dimethylethyl)-N-propylaminocarbonyl, N-butyl-N-(1-methylethyl)aminocarbonyl, N-(1-methylethyl)-N-(1-methylpropyl)aminocarbonyl, N-(1-methylethyl)-N-(2-methylpropyl)amino-carbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methyl-propyl)aminocarbonyl, N-(1,1-dimethylethyl)-N-(1-methylpropyl)aminocarbonyl or N-(1,1-dimethylethyl)-N-(2-methylpropyl)aminocarbonyl;
(C₁-C₆-alkylamino)carbonyl and also the (C₁-C₆-alkylamino)carbonyl moieties of (C₁-C₆-alkylamino)carbonylamino: (C₁-C₄-alkylamino)carbonyl as mentioned above, and also, for example, pentylaminocarbonyl, 1-methylbutylaminocarbonyl, 2-methyl-butylaminocarbonyl, 3-methylbutylaminocarbonyl, 2,2-dimethylpropylaminocarbonyl, 1-ethylpropylaminocarbonyl, hexylaminocarbonyl, 1,1-dimethylpropylaminocarbonyl, 1,2-dimethylpropylaminocarbonyl, 1-methylpentylaminocarbonyl, 2-methylpentyl-aminocarbonyl, 3-methylpentylaminocarbonyl, 4-methylpentylaminocarbonyl, 1,1-dimethylbutylaminocarbonyl, 1,2-dimethylbutylaminocarbonyl, 1,3-dimethylbutyl-aminocarbonyl, 2,2-dimethylbutylaminocarbonyl, 2,3-dimethylbutylaminocarbonyl, 3,3-dimethylbutylaminocarbonyl, 1-ethylbutylaminocarbonyl, 2-ethylbutylamino-carbonyl, 1,1,2-trimethylpropylaminocarbonyl, 1,2,2-trimethylpropylaminocarbonyl, 1-ethyl-1-methylpropylaminocarbonyl or 1-ethyl-2-methylpropylaminocarbonyl;
di-(C₁-C₆-alkyl)aminocarbonyl and also the di-(C₁-C₆-alkyl)aminocarbonyl moieties of di-(C₁-C₆-alkyl)aminocarbonylamino: di-(C₁-C₄-alkyl)aminocarbonyl as mentioned above, and also, for example, N-methyl-N-pentylaminocarbonyl, N-methyl-N-(1-methylbutyl)aminocarbonyl, N-methyl-N-(2-methylbutyl)aminocarbonyl, N-methyl-N-(3-methylbutyl)aminocarbonyl, N-methyl-N-(2,2-dimethylpropyl)amino-carbonyl, N-methyl-N-(1-ethylpropyl)aminocarbonyl, N-methyl-N-hexylamino-carbonyl, N-methyl-N-(1,1-dimethylpropyl)aminocarbonyl, N-methyl-N-(1,2-dimethyl-propyl)aminocarbonyl, N-methyl-N-(1-methylpentyl)aminocarbonyl, N-methyl-N-(2-methylpentyl)aminocarbonyl, N-methyl-N-(3-methylpentyl)aminocarbonyl, N-methyl-N-(4-methylpentyl)aminocarbonyl, N-methyl-N-(1,1-dimethylbutyl)amino-carbonyl, N-methyl-N-(1,2-dimethylbutyl)aminocarbonyl, N-methyl-N-(1,3-dimethyl-butyl)aminocarbonyl, N-methyl-N-(2,2-dimethylbutyl)aminocarbonyl, N-methyl-N-(2,3-dimethylbutyl)aminocarbonyl, N-methyl-N-(3,3-dimethylbutyl)aminocarbonyl, N-methyl-N-(1-ethylbutyl)aminocarbonyl, N-methyl-N-(2-ethylbutyl)aminocarbonyl, N-methyl-N-(1,1,2-trimethylpropyl)aminocarbonyl, N-methyl-N-(1,2,2-trimethyl-propyl)aminocarbonyl, N-methyl-N-(1-ethyl-1-methylpropyl)aminocarbonyl, N-methyl-N-(1-ethyl-2-methylpropyl)aminocarbonyl, N-ethyl-N-pentylaminocarbonyl, N-ethyl-N-(1-methylbutyl)aminocarbonyl, N-ethyl-N-(2-methylbutyl)aminocarbonyl, N-ethyl-N-(3-methylbutyl)aminocarbonyl, N-ethyl-N-(2,2-dimethylpropyl)amino-carbonyl, N-ethyl-N-(1-ethylpropyl)aminocarbonyl, N-ethyl-N-hexylaminocarbonyl, N-ethyl-N-(1,1-dimethylpropyl)aminocarbonyl, N-ethyl-N-(1,2-dimethylpropyl)amino-carbonyl, N-ethyl-N-(1-methylpentyl)aminocarbonyl, N-ethyl-N-(2-methylpentyl)-aminocarbonyl, N-ethyl-N-(3-methylpentyl)aminocarbonyl, N-ethyl-N-(4-methyl-pentyl)aminocarbonyl, N-ethyl-N-(1,1-dimethylbutyl)aminocarbonyl, N-ethyl-N-(1,2-dimethylbutyl)aminocarbonyl, N-ethyl-N-(1,3-dimethylbutyl)aminocarbonyl, N-ethyl-N-(2,2-dimethylbutyl)aminocarbonyl, N-ethyl-N-(2,3-dimethylbutyl)amino-carbonyl, N-ethyl-N-(3,3-dimethylbutyl)aminocarbonyl, N-ethyl-N-(1-ethylbutyl)-aminocarbonyl, N-ethyl-N-(2-ethylbutyl)aminocarbonyl, N-ethyl-N-(1,1,2-trimethyl-propyl)aminocarbonyl, N-ethyl-N-(1,2,2-trimethylpropyl)aminocarbonyl, N-ethyl-N-(1-ethyl-1-methylpropyl)aminocarbonyl, N-ethyl-N-(1-ethyl-2-methylpropyl)-aminocarbonyl, N-propyl-N-pentylaminocarbonyl, N-butyl-N-pentylaminocarbonyl, N,N-dipentylaminocarbonyl, N-propyl-N-hexylaminocarbonyl, N-butyl-N-hexylamino-carbonyl, N-pentyl-N-hexylaminocarbonyl or N,N-dihexylaminocarbonyl;
di-(C₁-C₆-alkyl)aminothiocarbonyl: for example N,N-dimethylaminothiocarbonyl, N,N-diethylaminothiocarbonyl, N,N-di-(1-methylethyl)aminothiocarbonyl, N,N-dipropylaminothiocarbonyl, N,N-dibutylaminothiocarbonyl, N,N-di-(1-methyl-propyl)aminothiocarbonyl, N,N-di-(2-methylpropyl)aminothiocarbonyl, N,N-di-(1,1-dimethylethyl)aminothiocarbonyl, N-ethyl-N-methylaminothiocarbonyl, N-methyl-N-propylaminothiocarbonyl, N-methyl-N-(1-methylethyl)aminothiocarbonyl, N-butyl-N-methylaminothiocarbonyl, N-methyl-N-(1-methylpropyl)aminothiocarbonyl, N-methyl-N-(2-methylpropyl)aminothiocarbonyl, N-(1,1-dimethylethyl)-N-methyl-aminothiocarbonyl, N-ethyl-N-propylaminothiocarbonyl, N-ethyl-N-(1-methylethyl)-aminothiocarbonyl, N-butyl-N-ethylaminothiocarbonyl, N-ethyl-N-(1-methylpropyl)-aminothiocarbonyl, N-ethyl-N-(2-methylpropyl)aminothiocarbonyl, N-ethyl-N-(1,1-dimethylethyl)aminothiocarbonyl, N-(1-methylethyl)-N-propylaminothio-carbonyl, N-butyl-N-propylaminothiocarbonyl, N-(1-methylpropyl)-N-propylamino-thiocarbonyl, N-(2-methylpropyl)-N-propylamino-thiocarbonyl, N-(1,1-dimethylethyl)-N-propylaminothiocarbonyl, N-butyl-N-(1-methylethyl)aminothiocarbonyl, N-(1-methylethyl)-N-(1-methylpropyl)aminothiocarbonyl, N-(1-methylethyl)-N-(2-methylpropyl)aminothiocarbonyl, N-(1,1-dimethylethyl)-N-(1-methylethyl)amino-thiocarbonyl, N-butyl-N-(1-methylpropyl)aminothiocarbonyl, N-butyl-N-(2-methyl-propyl)aminothiocarbonyl, N-butyl-N-(1,1-dimethylethyl)aminothiocarbonyl, N-(1-methylpropyl)-N-(2-methylpropyl)aminothiocarbonyl, N-(1,1-dimethylethyl)-N-(1-methylpropyl)aminothiocarbonyl, N-(1,1-dimethylethyl)-N-(2-methylpropyl)-aminothiocarbonyl, N-methyl-N-pentylaminothiocarbonyl, N-methyl-N-(1-methyl-butyl)aminothiocarbonyl, N-methyl-N-(2-methylbutyl)aminothiocarbonyl, N-methyl-N-(3-methylbutyl)aminothiocarbonyl, N-methyl-N-(2,2-dimethylpropyl)amino-thiocarbonyl, N-methyl-N-(1-ethylpropyl)aminothiocarbonyl, N-methyl-N-hexylamino-thiocarbonyl, N-methyl-N-(1,1-dimethylpropyl)aminothiocarbonyl, N-methyl-N-(1,2-dimethylpropyl)aminothiocarbonyl, N-methyl-N-(1-methylpentyl)-aminothiocarbonyl, N-methyl-N-(2-methylpentyl)aminothiocarbonyl, N-methyl-N-(3-methylpentyl)aminothiocarbonyl, N-methyl-N-(4-methylpentyl)aminothio-carbonyl, N-methyl-N-(1,1-dimethylbutyl)aminothiocarbonyl, N-methyl-N-(1,2-dimethylbutyl)aminothiocarbonyl, N-methyl-N-(1,3-dimethylbutyl)amino-thiocarbonyl, N-methyl-N-(2,2-dimethylbutyl)aminothiocarbonyl, N-methyl-N-(2,3-dimethylbutyl)aminothiocarbonyl, N-methyl-N-(3,3-dimethylbutyl)aminothio-carbonyl, N-methyl-N-(1-ethylbutyl)aminothiocarbonyl, N-methyl-N-(2-ethylbutyl)-aminothiocarbonyl, N-methyl-N-ethyl-N-(1,1,2-trimethylpropyl)aminothiocarbonyl, N-methyl-N-(1,2,2-trimethylpropyl)aminothiocarbonyl, N-methyl-N-(1-ethyl-1-methyl-propyl)aminothiocarbonyl, N-methyl-N-(1-ethyl-2-methylpropyl)aminothiocarbonyl, N-ethyl-N-pentylaminothiocarbonyl, N-ethyl-N-(1-methylbutyl)aminothiocarbonyl, N-ethyl-N-(2-methylbutyl)aminothiocarbonyl, N-ethyl-N-(3-methylbutyl)aminothio-carbonyl, N-ethyl-N-(2,2-dimethylpropyl)aminothiocarbonyl, N-ethyl-N-(1-ethyl-propyl)aminothiocarbonyl, N-ethyl-N-hexylaminothiocarbonyl, N-ethyl-N-(1,1-dimethylpropyl)aminothiocarbonyl, N-ethyl-N-(1,2-dimethylpropyl)amino-thiocarbonyl, N-ethyl-N-(1-methylpentyl)aminothiocarbonyl, N-ethyl-N-(2-methyl-pentyl)aminothiocarbonyl, N-ethyl-N-(3-methylpentyl)aminothiocarbonyl, N-ethyl-N-(4-methylpentyl)aminothiocarbonyl, N-ethyl-N-(1,1-dimethylbutyl)aminothio-carbonyl, N-ethyl-N-(1,2-dimethylbutyl)aminothiocarbonyl, N-ethyl-N-(1,3-dimethyl-butyl)aminothiocarbonyl, N-ethyl-N-(2,2-dimethylbutyl)aminothiocarbonyl, N-ethyl-N-(2,3-dimethylbutyl)aminothiocarbonyl, N-ethyl-N-(3,3-dimethylbutyl)amino-thiocarbonyl, N-ethyl-N-(1-ethylbutyl)aminothiocarbonyl, N-ethyl-N-(2-ethylbutyl)-aminothiocarbonyl, N-ethyl-N-(1,1,2-trimethylpropyl)aminothiocarbonyl, N-ethyl-N-(1,2,2-trimethylpropyl)aminothiocarbonyl, N-ethyl-N-(1-ethyl-1-methylpropyl)-aminothiocarbonyl, N-ethyl-N-(1-ethyl-2-methylpropyl)aminothiocarbonyl, N-propyl-N-pentylaminothiocarbonyl, N-butyl-N-pentylaminothiocarbonyl, N,N-dipentyl-aminothiocarbonyl, N-propyl-N-hexylaminothiocarbonyl, N-butyl-N-hexylaminothio-carbonyl, N-pentyl-N-hexylaminothiocarbonyl or N,N-dihexylaminothiocarbonyl;
heterocyclyl and the heterocyclyl moieties of heterocyclyl-C₁-C₆-alkyl, heterocyclyl-carbonyl, heterocyclylcarbonyl-C₁-C₆-alkyl, heterocyclyloxycarbonyl, heterocyclyl-aminocarbonyl, heterocyclylsulfonylaminocarbonyl, N—(C₁-C₆-alkyl)-N-(heterocyclyl)-aminocarbonyl and heterocyclyl-C₁-C₆-alkylcarbonyl:
a saturated, partially unsaturated or aromatic 5- or 6-membered heterocyclic ring which contains one to four identical or different heteroatoms selected from the group consisting of oxygen, sulfur and nitrogen and which may be attached via carbon or nitrogen, for example
5-membered saturated rings attached via carbon, such as: tetrahydrofuran-2-yl, tetrahydrofuran-3-yl, tetrahydrothien-2-yl, tetrahydrothien-3-yl, tetrahydropyrrol-2-yl, tetrahydropyrrol-3-yl, tetrahydropyrazol-3-yl, tetrahydropyrazol-4-yl, tetrahydro-isoxazol-3-yl, tetrahydroisoxazol-4-yl, tetrahydroisoxazol-5-yl, 1,2-oxathiolan-3-yl, 1,2-oxathiolan-4-yl, 1,2-oxathiolan-5-yl, tetrahydroisothiazol-3-yl, tetrahydro-isothiazol-4-yl, tetrahydroisothiazol-5-yl, 1,2-dithiolan-3-yl, 1,2-dithiolan-4-yl, tetrahydroimidazol-2-yl, tetrahydroimidazol-4-yl, tetrahydrooxazol-2-yl, tetrahydrooxazol-4-yl, tetrahydrooxazol-5-yl, tetrahydrothiazol-2-yl, tetra-hydrothiazol-4-yl, tetrahydrothiazol-5-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-dithiolan-2-yl, 1,3-dithiolan-4-yl, 1,3,2-dioxathiolan-4-yl;
5-membered saturated rings which are attached via nitrogen, such as: tetrahydro-pyrrol-1-yl, tetrahydropyrazol-1-yl, tetrahydroisoxazol-2-yl, tetrahydroisothiazol-2-yl, tetrahydroimidazol-1-yl, tetrahydrooxazol-3-yl, tetrahydrothiazol-3-yl;
5-membered partially unsaturated rings which are attached via carbon, such as: 2,3-dihydrofuran-2-yl, 2,3-dihydrofuran-3-yl, 2,5-dihydrofuran-2-yl, 2,5-dihydrofuran-3-yl, 4,5-dihydrofuran-2-yl, 4,5-dihydrofuran-3-yl, 2,3-dihydrothien-2-yl, 2,3-dihydro-thien-3-yl, 2,5-dihydrothien-2-yl, 2,5-dihydrothien-3-yl, 4,5-dihydrothien-2-yl, 4,5-dihydrothien-3-yl, 2,3-dihydro-1H-pyrrol-2-yl, 2,3-dihydro-1H-pyrrol-3-yl, 2,5-dihydro-1H-pyrrol-2-yl, 2,5-dihydro-1H-pyrrol-3-yl, 4,5-dihydro-1H-pyrrol-2-yl, 4,5-dihydro-1H-pyrrol-3-yl, 3,4-dihydro-2H-pyrrol-2-yl, 3,4-dihydro-2H-pyrrol-3-yl, 3,4-dihydro-5H-pyrrol-2-yl, 3,4-dihydro-5H-pyrrol-3-yl, 4,5-dihydro-1H-pyrazol-3-yl, 4,5-dihydro-1H-pyrazol-4-yl, 4,5-dihydro-1H-pyrazol-5-yl, 2,5-dihydro-1H-pyrazol-3-yl, 2,5-dihydro-1H-pyrazol-4-yl, 2,5-dihydro-1H-pyrazol-5-yl, 4,5-dihydroisoxazol-3-yl, 4,5-dihydroisoxazol-4-yl, 4,5-dihydroisoxazol-5-yl, 2,5-dihydroisoxazol-3-yl, 2,5-dihydroisoxazol-4-yl, 2,5-dihydroisoxazol-5-yl, 2,3-dihydroisoxazol-3-yl, 2,3-dihydroisoxazol-4-yl, 2,3-dihydroisoxazol-5-yl, 4,5-dihydroisothiazol-3-yl, 4,5-dihydroisothiazol-4-yl, 4,5-dihydroisothiazol-5-yl, 2,5-dihydroisothiazol-3-yl, 2,5-dihydroisothiazol-4-yl, 2,5-dihydroisothiazol-5-yl, 2,3-dihydroisothiazol-3-yl, 2,3-dihydroisothiazol-4-yl, 2,3-dihydroisothiazol-5-yl, Δ³-1,2-dithiol-3-yl, Δ³-1,2-dithiol-4-yl, Δ³-1,2-dithiol-5-yl, 4,5-dihydro-1H-imidazol-2-yl, 4,5-dihydro-1H-imidazol-4-yl, 4,5-dihydro-1H-imidazol-5-yl, 2,5-dihydro-1H-imidazol-2-yl, 2,5-dihydro-1H-imidazol-4-yl, 2,5-dihydro-1H-imidazol-5-yl, 2,3-dihydro-1H-imidazol-2-yl, 2,3-dihydro-1H-imidazol-4-yl, 4,5-dihydrooxazol-2-yl, 4,5-dihydrooxazol-4-yl, 4,5-dihydrooxazol-5-yl, 2,5-dihydrooxazol-2-yl, 2,5-dihydrooxazol-4-yl, 2,5-dihydro-oxazol-5-yl, 2,3-dihydrooxazol-2-yl, 2,3-dihydrooxazol-4-yl, 2,3-dihydrooxazol-5-yl, 4,5-dihydrothiazol-2-yl, 4,5-dihydrothiazol-4-yl, 4,5-dihydrothiazol-5-yl, 2,5-dihydro-thiazol-2-yl, 2,5-dihydrothiazol-4-yl, 2,5-dihydrothiazol-5-yl, 2,3-dihydrothiazol-2-yl, 2,3-dihydrothiazol-4-yl, 2,3-dihydrothiazol-5-yl, 1,3-dioxol-2-yl, 1,3-dioxol-4-yl, 1,3-dithiol-2-yl, 1,3-dithiol-4-yl, 1,3-oxathiol-2-yl, 1,3-oxathiol-4-yl, 1,3-oxathiol-5-yl, 1,2,3-Δ²-oxadiazolin-4-yl, 1,2,3-Δ²-oxadiazolin-5-yl, 1,2,4-Δ⁴-oxadiazolin-3-yl, 1,2,4-Δ⁴-oxadiazolin-5-yl, 1,2,4-Δ²-oxadiazolin-3-yl, 1,2,4-Δ²-oxadiazolin-5-yl, 1,2,4-Δ³-oxadiazolin-3-yl, 1,2,4-Δ³-oxadiazolin-5-yl, 1,3,4-Δ²-oxadiazolin-2-yl, 1,3,4-Δ²-oxadiazolin-5-yl, 1,3,4-Δ³-oxadiazolin-2-yl, 1,3,4-oxadiazolin-2-yl, 1,2,4-Δ⁴-thiadiazolin-3-yl, 1,2,4-Δ⁴-thiadiazolin-5-yl, 1,2,4-Δ³-thiadiazolin-3-yl, 1,2,4-Δ³-thiadiazolin-5-yl, 1,2,4-Δ²-thiadiazolin-3-yl, 1,2,4-Δ²-thiadiazolin-5-yl, 1,3,4-Δ²-thiadiazolin-2-yl, 1,3,4-Δ²-thiadiazolin-5-yl, 1,3,4-Δ³-thiadiazolin-2-yl, 1,3,4-thiadiazolin-2-yl, 1,2,3-Δ²-triazoline-4-yl, 1,2,3-Δ²-triazoline-5-yl, 1,2,4-Δ²-triazoline-3-yl, 1,2,4-Δ²-triazoline-5-yl, 1,2,4-Δ³-triazoline-3-yl, 1,2,4-Δ³-triazoline-5-yl, 1,2,4-Δ¹-triazoline-2-yl, 1,2,4-triazolin-3-yl, 3H-1,2,4-dithiazol-5-yl, 2H-1,3,4-dithiazol-5-yl, 2H-1,3,4-oxathiazol-5-yl;
5-membered partially unsaturated rings attached via nitrogen, such as: 2,3-dihydro-1H-pyrrol-1-yl, 2,5-dihydro-1H-pyrrol-1-yl, 4,5-dihydro-1H-pyrazol-1-yl, 2,5-dihydro-1H-pyrazol-1-yl, 2,3-dihydro-1H-pyrazol-1-yl, 2,5-dihydroisoxazol-2-yl, 2,3-dihydroisoxazol-2-yl, 2,5-dihydroisothiazol-2-yl, 2,3-dihydroisoxazol-2-yl, 4,5-dihydro-1H-imidazol-1-yl, 2,5-dihydro-1H-imidazol-1-yl, 2,3-dihydro-1H-imidazol-1-yl, 2,3-dihydrooxazol-3-yl, 2,3-dihydrothiazol-3-yl, 1,2,4-Δ⁴-oxadiazolin-2-yl, 1,2,4-Δ²-oxadiazolin-4-yl, 1,2,4-Δ³-oxadiazolin-2-yl, 1,3,4-Δ²-oxadiazolin-4-yl, 1,2,4-Δ⁵-thiadiazolin-2-yl, 1,2,4-Δ³-thiadiazolin-2-yl, 1,2,4-Δ²-thiadiazolin-4-yl, 1,3,4-Δ²-thiadiazolin-4-yl, 1,2,3-Δ²-triazolin-1-yl, 1,2,4-Δ²-triazolin-1-yl, 1,2,4-Δ²-triazolin-4-yl, 1,2,4-Δ³-triazolin-1-yl, 1,2,4-Δ²-triazolin-4-yl;
5-membered aromatic rings which are attached via carbon, such as: 2-furyl, 3-furyl, 2-thienyl, 3-thienyl, pyrrol-2-yl, pyrrol-3-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-2-yl, imidazol-4-yl, oxazol-2-yl, oxazol-4-yl, oxazol-5-yl, thiazol-2-yl, thiazol-4-yl, thiazol-5-yl, 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl, 1,2,3-triazol-4-yl, 1,2,4-triazol-3-yl, tetrazol-5-yl;
5-membered aromatic rings which are attached via nitrogen, such as: pyrrol-1-yl, pyrazol-1-yl, imidazol-1-yl, 1,2,3-triazol-1-yl, 1,2,4-triazol-1-yl, tetrazol-1-yl;
6-membered saturated rings which are attached via carbon, such as: tetrahydro-pyran-2-yl, tetrahydropyran-3-yl, tetrahydropyran-4-yl, piperidin-2-yl, piperidin-3-yl, piperidin-4-yl, tetrahydrothiopyran-2-yl, tetrahydrothiopyran-3-yl, tetrahydro-thiopyran-4-yl, 1,3-dioxan-2-yl, 1,3-dioxan-4-yl, 1,3-dioxan-5-yl, 1,4-dioxan-2-yl, 1,3-dithiane-2-yl, 1,3-dithiane-4-yl, 1,3-dithiane-5-yl, 1,4-dithiane-2-yl, 1,3-oxathiane-2-yl, 1,3-oxathiane-4-yl, 1,3-oxathiane-5-yl, 1,3-oxathiane-6-yl, 1,4-oxathiane-2-yl, 1,4-oxathiane-3-yl, 1,2-dithiane-3-yl, 1,2-dithiane-4-yl, hexahydropyrimidine-2-yl, hexahydro-pyrimidin-4-yl, hexahydropyrimidine-5-yl, hexahydropyrazine-2-yl, hexahydropyridazine-3-yl, hexahydropyridazine-4-yl, tetrahydro-1,3-oxazin-2-yl, tetrahydro-1,3-oxazin-4-yl, tetrahydro-1,3-oxazin-5-yl, tetrahydro-1,3-oxazin-6-yl, tetrahydro-1,3-thiazin-2-yl, tetrahydro-1,3-thiazin-4-yl, tetrahydro-1,3-thiazin-5-yl, tetrahydro-1,3-thiazin-6-yl, tetrahydro-1,4-thiazin-2-yl, tetrahydro-1,4-thiazin-3-yl, tetrahydro-1,4-oxazin-2-yl, tetrahydro-1,4-oxazin-3-yl, tetrahydro-1,2-oxazin-3-yl, tetrahydro-1,2-oxazin-4-yl, tetrahydro-1,2-oxazin-5-yl, tetrahydro-1,2-oxazin-6-yl;
6-membered saturated rings which are attached via nitrogen, such as: piperidin-1-yl, hexahydropyrimidine-1-yl, hexahydropyrazine-1-yl, hexahydropyridazine-1-yl, tetrahydro-1,3-oxazin-3-yl, tetrahydro-1,3-thiazin-3-yl, tetrahydro-1,4-thiazin-4-yl, tetrahydro-1,4-oxazin-4-yl, tetrahydro-1,2-oxazin-2-yl;
6-membered partially unsaturated rings which are attached via carbon, such as: 2H-3,4-dihydropyran-6-yl, 2H-3,4-dihydropyran-5-yl, 2H-3,4-dihydropyran-4-yl, 2H-3,4-dihydropyran-3-yl, 2H-3,4-dihydropyran-2-yl, 2H-3,4-dihydropyran-6-yl, 2H-3,4-dihydrothiopyran-5-yl, 2H-3,4-dihydrothiopyran-4-yl, 2H-3,4-dihydropyran-3-yl, 2H-3,4-dihydropyran-2-yl, 1,2,3,4-tetrahydropyridin-6-yl, 1,2,3,4-tetrahydro-pyridin-5-yl, 1,2,3,4-tetrahydropyridin-4-yl, 1,2,3,4-tetrahydropyridin-3-yl, 1,2,3,4-tetrahydropyridin-2-yl, 2H-5,6-dihydropyran-2-yl, 2H-5,6-dihydropyran-3-yl, 2H-5,6-dihydropyran-4-yl, 2H-5,6-dihydropyran-5-yl, 2H-5,6-dihydropyran-6-yl, 2H-5,6-dihydrothiopyran-2-yl, 2H-5,6-dihydrothiopyran-3-yl, 2H-5,6-dihydro-thiopyran-4-yl, 2H-5,6-dihydrothiopyran-5-yl, 2H-5,6-dihydrothiopyran-6-yl, 1,2,5,6-tetrahydropyridin-2-yl, 1,2,5,6-tetrahydropyridin-3-yl, 1,2,5,6-tetrahydro-pyridin-4-yl, 1,2,5,6-tetrahydropyridin-5-yl, 1,2,5,6-tetrahydropyridin-6-yl, 2,3,4,5-tetrahydropyridin-2-yl, 2,3,4,5-tetrahydropyridin-3-yl, 2,3,4,5-tetrahydro-pyridin-4-yl, 2,3,4,5-tetrahydropyridin-5-yl, 2,3,4,5-tetrahydropyridin-6-yl, 4H-pyran-2-yl, 4H-pyran-3-yl, 4H-pyran-4-yl, 4H-thiopyran-2-yl, 4H-thiopyran-3-yl, 4H-thio-pyran-4-yl, 1,4-dihydropyridine-2-yl, 1,4-dihydropyridine-3-yl, 1,4-dihydropyridine-4-yl, 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, 1,2-dihydropyridine-2-yl, 1,2-dihydropyridine-3-yl, 1,2-dihydro-pyridin-4-yl, 1,2-dihydropyridine-5-yl, 1,2-dihydropyridine-6-yl, 3,4-dihydropyridine-2-yl, 3,4-dihydropyridine-3-yl, 3,4-dihydropyridine-4-yl, 3,4-dihydropyridine-5-yl, 3,4-dihydro-pyridin-6-yl, 2,5-dihydropyridine-2-yl, 2,5-dihydropyridine-3-yl, 2,5-dihydropyridine-4-yl, 2,5-dihydropyridine-5-yl, 2,5-dihydropyridine-6-yl, 2,3-dihydropyridine-2-yl, 2,3-dihydro-pyridin-3-yl, 2,3-dihydropyridine-4-yl, 2,3-dihydropyridine-5-yl, 2,3-dihydropyridine-6-yl, 2H-5,6-dihydro-1,2-oxazin-3-yl, 2H-5,6-dihydro-1,2-oxazin-4-yl, 2H-5,6-dihydro-1,2-oxazin-5-yl, 2H-5,6-dihydro-1,2-oxazin-6-yl, 2H-5,6-dihydro-1,2-thiazin-3-yl, 2H-5,6-dihydro-1,2-thiazin-4-yl, 2H-5,6-dihydro-1,2-thiazin-5-yl, 2H-5,6-dihydro-1,2-thiazin-6-yl, 4H-5,6-dihydro-1,2-oxazin-3-yl, 4H-5,6-dihydro-1,2-oxazin-4-yl, 4H-5,6-dihydro-1,2-oxazin-5-yl, 4H-5,6-dihydro-1,2-oxazin-6-yl, 4H-5,6-dihydro-1,2-thiazin-3-yl, 4H-5,6-dihydro-1,2-thiazin-4-yl, 4H-5,6-dihydro-1,2-thiazin-5-yl, 4H-5,6-dihydro-1,2-thiazin-6-yl, 2H-3,6-dihydro-1,2-oxazin-3-yl, 2H-3,6-dihydro-1,2-oxazin-4-yl, 2H-3,6-dihydro-1,2-oxazin-5-yl, 2H-3,6-dihydro-1,2-oxazin-6-yl, 2H-3,6-dihydro-1,2-thiazin-3-yl, 2H-3,6-dihydro-1,2-thiazin-4-yl, 2H-3,6-dihydro-1,2-thiazin-5-yl, 2H-3,6-dihydro-1,2-thiazin-6-yl, 2H-3,4-dihydro-1,2-oxazin-3-yl, 2H-3,4-dihydro-1,2-oxazin-4-yl, 2H-3,4-dihydro-1,2-oxazin-5-yl, 2H-3,4-dihydro-1,2-oxazin-6-yl, 2H-3,4-dihydro-1,2-thiazin-3-yl, 2H-3,4-dihydro-1,2-thiazin-4-yl, 2H-3,4-dihydro-1,2-thiazin-5-yl, 2H-3,4-dihydro-1,2-thiazin-6-yl, 2,3,4,5-tetrahydro-pyridazin-3-yl, 2,3,4,5-tetrahydropyridazine-4-yl, 2,3,4,5-tetrahydropyridazine-5-yl, 2,3,4,5-tetrahydropyridazine-6-yl, 3,4,5,6-tetrahydropyridazine-3-yl, 3,4,5,6-tetra-hydropyridazin-4-yl, 1,2,5,6-tetrahydropyridazine-3-yl, 1,2,5,6-tetrahydropyridazine-4-yl, 1,2,5,6-tetrahydropyridazine-5-yl, 1,2,5,6-tetrahydropyridazine-6-yl, 1,2,3,6-tetra-hydropyridazin-3-yl, 1,2,3,6-tetrahydropyridazine-4-yl, 4H-5,6-dihydro-1,3-oxazin-2-yl, 4H-5,6-dihydro-1,3-oxazin-4-yl, 4H-5,6-dihydro-1,3-oxazin-5-yl, 4H-5,6-dihydro-1,3-oxazin-6-yl, 4H-5,6-dihydro-1,3-thiazin-2-yl, 4H-5,6-dihydro-1,3-thiazin-4-yl, 4H-5,6-dihydro-1,3-thiazin-5-yl, 4H-5,6-dihydro-1,3-thiazin-6-yl, 3,4,5,6-tetrahydro-pyrimidin-2-yl, 3,4,5,6-tetrahydropyrimidin-4-yl, 3,4,5,6-tetrahydropyrimidin-5-yl, 3,4,5,6-tetrahydropyrimidin-6-yl, 1,2,3,4-tetrahydropyrazine-2-yl, 1,2,3,4-tetrahydro-pyrazin-5-yl, 1,2,3,4-tetrahydro-pyrimidin-2-yl, 1,2,3,4-tetrahydropyrimidin-4-yl, 1,2,3,4-tetrahydropyrimidin-5-yl, 1,2,3,4-tetrahydropyrimidin-6-yl, 2,3-dihydro-1,4-thiazin-2-yl, 2,3-dihydro-1,4-thiazin-3-yl, 2,3-dihydro-1,4-thiazin-5-yl, 2,3-dihydro-1,4-thiazin-6-yl, 2H-1,2-oxazin-3-yl, 2H-1,2-oxazin-4-yl, 2H-1,2-oxazin-5-yl, 2H-1,2-oxazin-6-yl, 2H-1,2-thiazin-3-yl, 2H-1,2-thiazin-4-yl, 2H-1,2-thiazin-5-yl, 2H-1,2-thiazin-6-yl, 4H-1,2-oxazin-3-yl, 4H-1,2-oxazin-4-yl, 4H-1,2-oxazin-5-yl, 4H-1,2-oxazin-6-yl, 4H-1,2-thiazin-3-yl, 4H-1,2-thiazin-4-yl, 4H-1,2-thiazin-5-yl, 4H-1,2-thiazin-6-yl, 6H-1,2-oxazin-3-yl, 6H-1,2-oxazin-4-yl, 6H-1,2-oxazin-5-yl, 6H-1,2-oxazin-6-yl, 6H-1,2-thiazin-3-yl, 6H-1,2-thiazin-4-yl, 6H-1,2-thiazin-5-yl, 6H-1,2-thiazin-6-yl, 2H-1,3-oxazin-2-yl, 2H-1,3-oxazin-4-yl, 2H-1,3-oxazin-5-yl, 2H-1,3-oxazin-6-yl, 2H-1,3-thiazin-2-yl, 2H-1,3-thiazin-4-yl, 2H-1,3-thiazin-5-yl, 2H-1,3-thiazin-6-yl, 4H-1,3-oxazin-2-yl, 4H-1,3-oxazin-4-yl, 4H-1,3-oxazin-5-yl, 4H-1,3-oxazin-6-yl, 4H-1,3-thiazin-2-yl, 4H-1,3-thiazin-4-yl, 4H-1,3-thiazin-5-yl, 4H-1,3-thiazin-6-yl, 6H-1,3-oxazin-2-yl, 6H-1,3-oxazin-4-yl, 6H-1,3-oxazin-5-yl, 6H-1,3-oxazin-6-yl, 6H-1,3-thiazin-2-yl, 6H-1,3-oxazin-4-yl, 6H-1,3-oxazin-5-yl, 6H-1,3-thiazin-6-yl, 2H-1,4-oxazin-2-yl, 2H-1,4-oxazin-3-yl, 2H-1,4-oxazin-5-yl, 2H-1,4-oxazin-6-yl, 2H-1,4-thiazin-2-yl, 2H-1,4-thiazin-3-yl, 2H-1,4-thiazin-5-yl, 2H-1,4-thiazin-6-yl, 4H-1,4-oxazin-2-yl, 4H-1,4-oxazin-3-yl, 4H-1,4-thiazin-2-yl, 4H-1,4-thiazin-3-yl, 1,4-dihydropyridazine-3-yl, 1,4-dihydropyridazine-4-yl, 1,4-dihydro-pyridazin-5-yl, 1,4-dihydropyridazine-6-yl, 1,4-dihydropyrazine-2-yl, 1,2-dihydro-pyrazin-2-yl, 1,2-dihydropyrazine-3-yl, 1,2-dihydropyrazine-5-yl, 1,2-dihydropyrazine-6-yl, 1,4-dihydropyrimidine-2-yl, 1,4-dihydropyrimidine-4-yl, 1,4-dihydropyrimidine-5-yl, 1,4-dihydropyrimidine-6-yl, 3,4-dihydropyrimidine-2-yl, 3,4-dihydropyrimidine-4-yl, 3,4-dihydropyrimidine-5-yl or 3,4-dihydropyrimidine-6-yl;
6-membered partially unsaturated rings which are attached via nitrogen, such as: 1,2,3,4-tetrahydropyridin-1-yl, 1,2,5,6-tetrahydropyridin-1-yl, 1,4-dihydropyridine-1-yl, 1,2-dihydropyridine-1-yl, 2H-5,6-dihydro-1,2-oxazin-2-yl, 2H-5,6-dihydro-1,2-thiazin-2-yl, 2H-3,6-dihydro-1,2-oxazin-2-yl, 2H-3,6-dihydro-1,2-thiazin-2-yl, 2H-3,4-dihydro-1,2-oxazin-2-yl, 2H-3,4-dihydro-1,2-thiazin-2-yl, 2,3,4,5-tetrahydropyridazine-2-yl, 1,2,5,6-tetrahydropyridazine-1-yl, 1,2,5,6-tetrahydropyridazine-2-yl, 1,2,3,6-tetrahydro-pyridazin-1-yl, 3,4,5,6-tetrahydropyrimidin-3-yl, 1,2,3,4-tetrahydropyrazine-1-yl, 1,2,3,4-tetrahydropyrimidin-1-yl, 1,2,3,4-tetrahydropyrimidin-3-yl, 2,3-dihydro-1,4-thiazin-4-yl, 2H-1,2-oxazin-2-yl, 2H-1,2-thiazin-2-yl, 4H-1,4-oxazin-4-yl, 4H-1,4-thiazin-4-yl, 1,4-dihydropyridazine-1-yl, 1,4-dihydropyrazine-1-yl, 1,2-dihydro-pyrazin-1-yl, 1,4-dihydropyrimidine-1-yl or 3,4-dihydropyrimidine-3-yl;
6-membered aromatic rings which are attached via carbon, such as: pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl, 1,2,4-triazin-5-yl, 1,2,4-triazin-6-yl, 1,2,4,5-tetrazine-3-yl;
it being possible for a bicyclic ring system to be formed with a fused-on phenyl ring or with a C₃-C₆-carbocycle or a further 5- or 6-membered heterocycle.
Aryl and the aryl moiety of aryl-(C₁-C₆-alkyl): a monocyclic to tricyclic aromatic carbocycle having 6 to 14 ring members, such as, for example, phenyl, naphthyl and anthracenyl;
5- or 6-membered heteroaryl having one to four nitrogen atoms or one to three nitrogen atoms and one oxygen or sulfur atom or having one oxygen or sulfur atom: for example aromatic 5-membered heterocycles which are attached via a carbon atom and which, in addition to carbon atoms, may contain one to four nitrogen atoms or one to three nitrogen atoms and one sulfur or oxygen atom or one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; for example aromatic 6-membered heterocycles which are attached via a carbon atom and which, in addition to carbon atoms, may contain one to four, preferably one to three, nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;
mono- or bicyclic heteroaryl having 5 to 10 ring members and containing 1 to 4 heteroatoms from the group consisting of nitrogen, oxygen and sulfur: mono- or bicyclic aromatic heteroaryl having 5 to 10 ring members which, in addition to carbon atoms, contains 1 to 4 nitrogen atoms or 1 to 3 nitrogen atoms and one oxygen or one sulfur atom or one oxygen or one sulfur atom, for example monocycles, such as furyl (for example 2-furyl, 3-furyl), thienyl (for example 2-thienyl, 3-thienyl), pyrrolyl (for example pyrrol-2-yl, pyrrol-3-yl), pyrazolyl (for example pyrazol-3-yl, pyrazol-4-yl), isoxazolyl (for example isoxazol-3-yl, isoxazol-4-yl, isoxazol-5-yl), isothiazolyl (for example isothiazol-3-yl, isothiazol-4-yl, isothiazol-5-yl), imidazolyl (for example imidazol-2-yl, imidazol-4-yl), oxazolyl (for example oxazol-2-yl, oxazol-4-yl, oxazol-5-yl), thiazolyl (for example thiazol-2-yl, thiazol-4-yl, thiazol-5-yl), oxadiazolyl (for example 1,2,3-oxadiazol-4-yl, 1,2,3-oxa-diazol-5-yl, 1,2,4-oxadiazol-3-yl, 1,2,4,-oxadiazol-5-yl, 1,3,4-oxadiazol-2-yl), thiadiazolyl (for example 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl), triazolyl (for example 1, 2,3-triazol-4-yl, 1,2,4-triazol-3-yl), tetrazol-5-yl, pyridyl (for example pyridin-2-yl, pyridin-3-yl, pyridin-4-yl), pyrazinyl (for example pyridazin-3-yl, pyridazin-4-yl), pyrimidinyl (for example pyrimidin-2-yl, pyrimidin-4-yl, pyrimidin-5-yl), pyrazin-2-yl, triazinyl (for example 1, 3,5-triazin-2-yl, 1,2,4-triazin-3-yl, 1,2,4-triazin-5-yl, 1,2,4-triazin-6-yl), tetrazineyl (for example 1,2,4,5-tetrazine-3-yl); and also bicycles, such as the benzo-fused derivatives of the abovementioned monocycles, for example quinolinyl, isoquinolinyl, indolyl, benzothienyl, benzofuranyl, benzoxazolyl, benzothiazolyl, benzisothiazolyl, benzimidazolyl, benzopyrazolyl, benzothiadiazolyl, benzotriazolyl.

All phenyl and aryl rings or heterocyclyl and heteroaryl radicals and all phenyl components in phenyl-C₁-C₆-alkyl, phenylcarbonyl, phenylcarbonyl-C₁-C₆-alkyl, phenoxycarbonyl, phenylaminocarbonyl, phenylsulfonylaminocarbonyl, N—(C₁-C₆-alkyl)-N-phenylaminocarbonyl and phenyl-C₁-C₆-alkylcarbonyl, all aryl components in aryl(C₁-C₄-alkyl), all heteroaryl components in mono- or bicyclic heteroaryl and all heterocyclyl components in heterocyclyl-C₁-C₆-alkyl, heterocyclylcarbonyl, heterocyclylcarbonyl-C₁-C₆-alkyl, heterocyclyloxycarbonyl, heterocyclylaminocarbonyl, heterocyclylsulfonylaminocarbonyl, N—(C₁-C₆-alkyl)-N-heterocyclylaminocarbonyl and heterocyclyl-C₁-C₆-alkylcarbonyl are, unless indicated otherwise, preferably unsubstituted or carry one to three halogen atoms and/or one nitro group, one cyano radical and/or one or two methyl, trifluoromethyl, methoxy or trifluoromethoxy substituents.

In a particular embodiment, the variables of the heteroaroyl-substituted serineamides of the formula I are as defined below, these definitions being, both on their own and in combination with one another, particular embodiments of the compounds of the formula I:

Preference is given to the heteroaroyl-substituted serineamides of the formula I in which
A is 5-membered heteroaryl having one to four nitrogen atoms or one to three nitrogen atoms and one oxygen or sulfur atom or having one oxygen or sulfur atom;
   particularly preferably 5-membered heteroaryl selected from the group consisting of thienyl, furyl, pyrazolyl, imidazolyl, thiazolyl and oxazolyl;
   especially preferably 5-membered heteroaryl selected from the group consisting of thienyl, furyl, pyrazolyl and imidazolyl;
   where the heteroaryl radicals mentioned may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl.

Preference is also given to the heteroaroyl-substituted serineamides of the formula I in which
A is 6-membered heteroaryl having one to four nitrogen atoms;
particularly preferably pyridyl or pyrimidyl;
especially preferably pyrimidyl;

where the heteroaryl radicals mentioned may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl.

Preference is also given to the heteroaroyl-substituted serineamides of the formula I in which
A is 5- or 6-membered heteroaryl selected from the group consisting of pyrrolyl, thienyl, furyl, pyrazolyl, imidazolyl, thiazolyl, oxazolyl, tetrazolyl, pyridyl and pyrimidinyl; where the heteroaryl radicals mentioned may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
   particularly preferably 5- or 6-membered heteroaryl selected from the group consisting of thienyl, furyl, pyrazolyl, imidazolyl, thiazolyl, oxazolyl and pyridyl; where the heteroaryl radicals mentioned may be partially or fully halogenated and/or may carry 1 to 3 radicals from the group consisting of C₁-C₆-alkyl, C₃-C₆-cycloalkyl and C₁-C₆-haloalkyl;
   especially preferably 5-membered heteroaryl selected from the group consisting of thienyl, furyl, pyrazolyl, imidazolyl, thiazolyl and oxazolyl; where the heteroaryl radicals mentioned may be partially halogenated and/or may carry 1 to 2 radicals from the group consisting of C₁-C₆-alkyl and C₁-C₄-haloalkyl;
   most preferably 5-membered heteroaryl selected from the group consisting of thienyl, furyl, pyrazolyl and imidazolyl; where the heteroaryl radicals mentioned may be partially halogenated and/or may carry 1 to 2 radicals from the group consisting of C₁-C₆-alkyl and C₁-C₄-haloalkyl.

Preference is also given to the heteroaroyl-substituted serineamides of the formula I in which
A is 5- or 6-membered heteroaryl which is attached via carbon and selected from the group consisting of A1 to A14 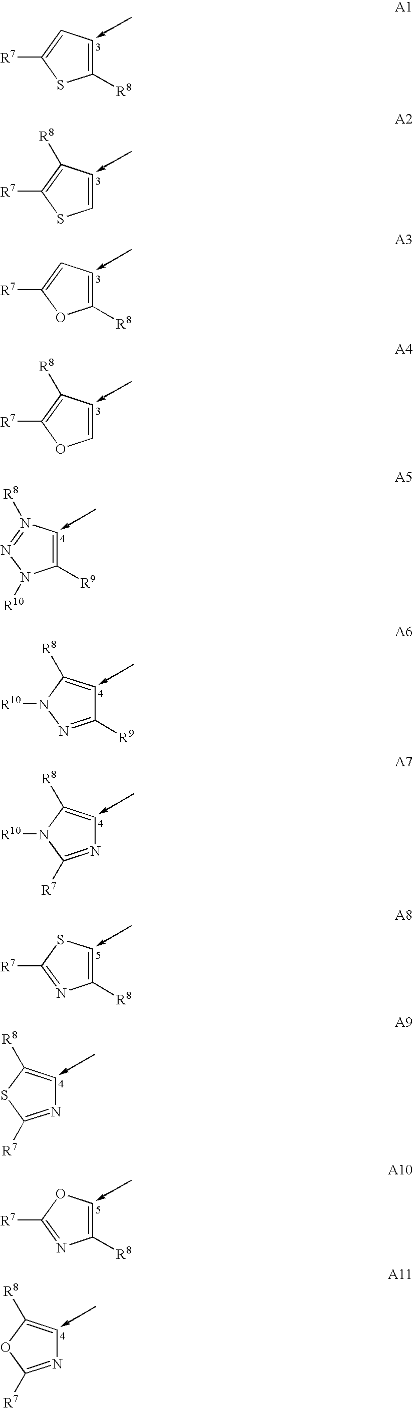 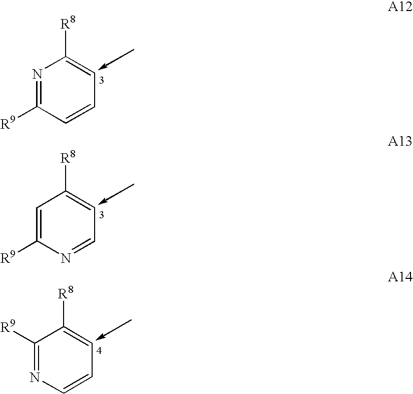
   where the arrow indicates the point of attachment and
      R⁷ is hydrogen, halogen, C₁-C₆-alkyl or C₁-C₆-haloalkyl;
      particularly preferably hydrogen, C₁-C₄-alkyl or C₁-C₄-haloalkyl; especially preferably hydrogen or C₁-C₄-alkyl;
      most preferably hydrogen;
      R⁸ is halogen, C₁-C₆-alkyl, C₁-C₆-haloalkyl or C₁-C₆-haloalkoxy;
      particularly preferably halogen, C₁-C₄-alkyl or C₁-C₄-haloalkyl;
      especially preferably halogen or C₁-C₄-haloalkyl;
      most preferably CF₃;
      R⁹ is hydrogen, halogen, C₁-C₆-alkyl or C₁-C₆-haloalkyl;
      particularly preferably hydrogen, halogen or C₁-C₄-haloalkyl;
      especially preferably hydrogen or halogen;
      most preferably hydrogen; and
      R¹⁰ is hydrogen, C₁-C₆-alkyl, C₃-C₆-cycloalkyl, C₁-C₆-haloalkyl or C₁-C₆-alkoxy-C₁-C₄-alkyl;
      particularly preferably C₁-C₄-alkyl, C₃-C₆-cycloalkyl, C₁-C₄-haloalkyl or C₁-C₄-alkoxy-C₁-C₄-alkyl;
      especially preferably C₁-C₄-alkyl or C₁-C₄-haloalkyl;
      most preferably C₁-C₄-alkyl;
      with utmost preference CH₃;
   particularly preferably A1, A2, A3, A4, A5, A6, A8 or A9;
      where R⁷ to R⁹ are as defined above;
   most preferably A1, A2, A5 or A6;
      where R⁷ to R⁹ are as defined above.

Preference is given to the heteroaroyl-substituted serineamides of the formula I in which
Het is mono- or bicyclic heteroaryl having 5 to 10 ring members including 1 to 4 heteroatoms from the group consisting of nitrogen, oxygen and sulfur,
   which may be partially or fully halogenated and/or may carry 1 to 3 radicals from the group consisting of nitro, C₁-C₄-alkyl, C₁-C₄-haloalkyl, hydroxyl, C₁-C₄-alkoxy, C₁-C₄-haloalkoxy, hydroxycarbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)amino, C₁-C₄-alkylsulfonylamino, C₁-C₄-haloalkylsulfonylamino, aminocarbonylamino, (C₁-C₄-alkylamino)-carbonylamino and di-(C₁-C₄-alkyl)aminocarbonylamino;
particularly preferably mono- or bicyclic heteroaryl selected from the group consisting of furyl, thienyl, pyrrolyl, pyrazolyl, isoxazolyl, isothiazolyl, imidazolyl, oxazolyl, thiazolyl, oxadiazolyl, thiadiazolyl, triazolyl, tetrazolyl, pyridyl, pyridazinyl, pyrimidinyl, pyrazinyl, triazinyl, tetrazineyl, quinolinyl, isoquinolinyl, indolyl, benzothienyl, benzofuranyl, benzoxazolyl, benzothiazolyl, benzisothiazolyl, benzimidazolyl, benzopyrazolyl, benzothiadiazolyl and benzotriazolyl,
   where the heteroaryls mentioned may be partially or fully halogenated and/or may carry 1 to 3 radicals from the group consisting of nitro, C₁-C₄-alkyl, C₁-C₄-haloalkyl, hydroxyl, C₁-C₄-alkoxy, C₁-C₄-haloalkoxy, hydroxy-carbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)-amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)-aminocarbonylamino;
especially preferably mono- or bicyclic heteroaryl selected from the group consisting of furyl, thienyl, pyrazolyl, imidazolyl, thiazolyl, triazolyl, tetrazolyl, pyridyl, pyrimidinyl, quinolinyl and indolyl,
   where the heteroaryls mentioned may be partially or fully halogenated and/or may carry 1 to 3 radicals from the group consisting of nitro, C₁-C₄-alkyl, C₁-C₄-haloalkyl, hydroxyl, C₁-C₄-alkoxy, C₁-C₄-haloalkoxy, hydroxy-carbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)-amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)-aminocarbonylamino;
most preference is given to mono- or bicyclic heteroaryl selected from the group consisting of thienyl, thiazolyl, tetrazolyl, pyridyl and indolyl,
   where the heteroaryls mentioned may be partially or fully halogenated and/or may carry 1 to 2 radicals from the group consisting of nitro, C₁-C₄-alkyl, C₁-C₄-haloalkyl, hydroxyl, C₁-C₄-alkoxy, C₁-C₄-haloalkoxy, hydroxy-carbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)-amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)-aminocarbonylamino.

Particular preference is given to the heteroaroyl-substituted serineamides of the formula I in which Het is Het-1 to Het-6 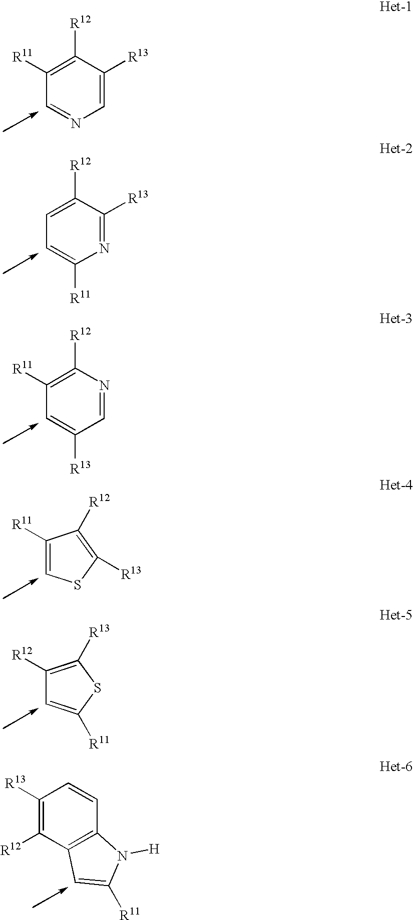
where the arrow indicates the point of attachment and
R¹¹ is hydrogen, halogen, C₁-C₆-alkyl or C₁-C₆-haloalkyl;
preferably hydrogen, halogen, C₁-C₄-alkyl or C₁-C₄-haloalkyl;
especially preferably hydrogen, halogen or C₁-C₄-alkyl;
particularly preferably hydrogen, fluorine, chlorine or methyl;
R¹² is hydrogen, halogen, C₁-C₆-alkyl or C₁-C₆-haloalkyl;
preferably hydrogen, halogen, C₁-C₄-alkyl or C₁-C₄-haloalkyl;
especially preferably hydrogen, halogen or C₁-C₄-alkyl;
particularly preferably hydrogen, fluorine, chlorine or methyl;
R¹³ is hydrogen, halogen or C₁-C₄-alkyl;
preferably hydrogen or halogen;
especially preferably hydrogen or fluorine.

Preference is likewise given to the heteroaroyl-substituted serineamides of the formula I in which
R¹ is hydrogen; and
R² is hydrogen or hydroxyl;
particularly preferably hydrogen.

Preference is likewise given to the heteroaroyl-substituted serine amides of the formula I in which
R³ is C₁-C₆-alkyl or C₁-C₆-haloalkyl;
particularly preferably C₁-C₆-alkyl;
especially preferably C₁-C₄-alkyl;
most preferably CH₃.

Preference is likewise given to the heteroaroyl-substituted serineamides of the formula I, in which
R⁴ is hydrogen, C₁-C₆-alkyl, C₃-C₆-alkenyl, C₃-C₆-alkynyl, C₁-C₆-alkylcarbonyl, C₂-C₆-alkenylcarbonyl, C₃-C₆-cycloalkylcarbonyl, C₁-C₆-alkoxycarbonyl, C₁-C₆-alkylaminocarbonyl, C₁-C₆-alkylsulfonylaminocarbonyl, di-(C₁-C₆-alkyl)amino-carbonyl, N—(C₁-C₆-alkoxy)-N—(C₁-C₆-alkyl)aminocarbonyl, di-(C₁-C₆-alkyl)-aminothiocarbonyl, C₁-C₆-alkoxyimino-C₁-C₆-alkyl,
   where the alkyl, cycloalkyl and alkoxy radicals mentioned may be partially or fully halogenated and/or may carry one to three of the following groups: cyano, hydroxyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl, or C₁-C₄-alkylcarbonyloxy;
phenyl, phenyl-C₁-C₆-alkyl, phenylcarbonyl, phenylcarbonyl-C₁-C₆-alkyl, phenylsulfonylaminocarbonyl or phenyl-C₁-C₆-alkylcarbonyl,
   where the phenyl radical of the 6 last-mentioned substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R⁶;
particularly preferably hydrogen, C₁-C₆-alkyl, C₃-C₆-alkenyl, C₃-C₆-alkynyl, C₁-C₆-alkylcarbonyl, C₂-C₆-alkenylcarbonyl, C₁-C₆-alkoxycarbonyl, C₁-C₆-alkyl-sulfonylaminocarbonyl, di-(C₁-C₆-alkyl)aminocarbonyl, N—(C₁-C₆-alkoxy)-N—(C₁-C₆-alkyl)aminocarbonyl or di-(C₁-C₆-alkyl)aminothiocarbonyl,
   where the alkyl or alkoxy radicals mentioned may be partially or fully halogenated and/or may carry one to three of the following groups: cyano, C₁-C₄-alkoxy, C₁-C₄-alkoxycarbonyl, C₁-C₄-alkylaminocarbonyl, di-(C₁-C₄-alkyl)aminocarbonyl or C₁-C₄-alkylcarbonyloxy;
phenyl-C₁-C₆-alkyl, phenylcarbonyl, phenylcarbonyl-C₁-C₆-alkyl, phenylsulfonyl-aminocarbonyl or phenyl-C₁-C₆-alkylcarbonyl,
   where the phenyl ring of the 5 last-mentioned substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R⁶;
especially preferably hydrogen, C₁-C₆-alkyl, C₃-C₆-alkenyl, C₃-C₆-alkynyl, C₁-C₆-alkylcarbonyl, C₂-C₆-alkenylcarbonyl, C₁-C₆-alkoxycarbonyl, di-(C₁-C₆-alkyl)-aminocarbonyl, N—(C₁-C₆-alkoxy)-N—(C₁-C₆-alkyl)aminocarbonyl, di-(C₁-C₆-alkyl)-aminothiocarbonyl, phenyl-C₁-C₆-alkyl, phenylcarbonyl, phenylcarbonyl-C₁-C₆-alkyl or phenyl-C₁-C₆-alkylcarbonyl
   where the phenyl ring of the 4 last-mentioned substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R⁶.

Preference is likewise given to the heteroaroyl-substituted serineamides of the formula I, in which
R⁴ is hydrogen, C₁-C₆-alkyl, C₃-C₆-alkenyl, C₃-C₆-alkynyl, C₁-C₆-alkylcarbonyl, C₂-C₆-alkenylcarbonyl, C₃-C₆-cycloalkylcarbonyl, C₁-C₆-alkoxycarbonyl, C₁-C₆-alkylaminocarbonyl, di-(C₁-C₆-alkyl)aminocarbonyl, N—(C₁-C₆-alkoxy)-N—(C₁-C₆-alkyl)aminocarbonyl, di-(C₁-C₆-alkyl)aminothiocarbonyl, C₁-C₆-alkoxyimino-C₁-C₆-alkyl,
   where the alkyl, cycloalkyl or alkoxy radicals mentioned may be partially or fully halogenated and/or may carry one to three of the following groups:

cyano, hydroxyl, C₃-C₆-Cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy; or
SO₂R⁶.

Preference is likewise given to the heteroaroyl-substituted serineamides of the formula I, in which
R⁴ is hydrogen, C₁-C₆-alkyl, C₃-C₆-alkenyl, C₃-C₆-alkynyl, C₁-C₆-alkylcarbonyl, C₁-C₆-alkoxycarbonyl, C₁-C₆-alkylaminocarbonyl, di-(C₁-C₆-alkyl)aminocarbonyl, N—(C₁-C₆-alkoxy)-N—(C₁-C₆-alkyl)aminocarbonyl,
   where the alkyl and alkoxy radicals mentioned may be partially or fully halogenated and/or may carry one to three of the following groups: cyano, C₁-C₄-alkoxy, C₁-C₄-alkylaminocarbonyl or di-(C₁-C₄-alkyl)aminocarbonyl;
phenyl-C₁-C₆-alkyl, phenylcarbonyl, phenylcarbonyl-C₁-C₆-alkyl, phenyl-aminocarbonyl, N—(C₁-C₆-alkyl)-N-(phenyl)aminocarbonyl or heterocyclylcarbonyl,
   where the phenyl and the heterocyclyl radical of the 6 last-mentioned substituents may be partially or fully halogenated and/or may carry one to three of the following groups: cyano, C₁-C₄-alkyl or C₁-C₄-haloalkyl; or
SO₂R⁶;
particularly preferably hydrogen, C₁-C₄-alkyl, C₃-C₄-alkenyl, C₃-C₄-alkynyl, C₁-C₄-alkylcarbonyl, C₁-C₄-alkoxycarbonyl, C₁-C₄-alkylaminocarbonyl, di-(C₁-C₄-alkyl)-aminocarbonyl, N—(C₁-C₄-alkoxy)-N—(C₁-C₄-alkyl)aminocarbonyl, especially preferably hydrogen or C₁-C₄-alkyl;
   where the alkyl and alkoxy radicals may be partially or fully halogenated and/or may carry one to three of the following groups: cyano, C₁-C₄-alkoxy, C₁-C₄-alkylaminocarbonyl or di-(C₁-C₄-alkyl)aminocarbonyl;
phenyl-C₁-C₄-alkyl, phenylcarbonyl, phenylcarbonyl-C₁-C₄-alkyl,
phenylaminocarbonyl, N—(C₁-C₄-alkyl)-N-(phenyl)aminocarbonyl or heterocyclylcarbonyl,
   where the phenyl and the heterocyclyl radical of the 6 last-mentioned substituents may be partially or fully halogenated and/or may carry one to three of the following groups: cyano, C₁-C₄-alkyl or C₁-C₄-haloalkyl; or
SO₂R⁶;
most preferably hydrogen, C₁-C₄-alkylcarbonyl, C₁-C₄-alkylaminocarbonyl, di-(C₁-C₄-alkyl)aminocarbonyl, phenylaminocarbonyl, N—(C₁-C₄-alkyl)-N-(phenyl)-aminocarbonyl, SO₂CH₃ or SO₂(C₆H₅).

Preference is likewise given to the heteroaroyl-substituted serineamides of the formula I, in which
R⁵ is hydrogen or C₁-C₄-alkyl;
preferably hydrogen or CH₃;
especially preferably hydrogen.

Preference is likewise given to the heteroaroyl-substituted serineamides of the formula I, in which
R⁶ is C₁-C₆-alkyl, C₁-C₆-haloalkyl or phenyl,
   where the phenyl radical may be partially or fully halogenated and/or may be substituted by C₁-C₄-alkyl;
particularly preferably C₁-C₄-alkyl, C₁-C₄-haloalkyl or phenyl;
especially preferably methyl, trifluoromethyl or phenyl.

Particular preference is given to the heteroaroyl-substituted serineamides of the formula I in which
A is 5- or 6-membered heteroaryl selected from the group consisting of thienyl, furyl, pyrazolyl, imidazolyl, thiazolyl, oxazolyl and pyridyl;
   where the heteroaryl radicals mentioned may be partially or fully halogenated and/or may carry 1 to 3 radicals from the group consisting of C₁-C₆-alkyl, C₃-C₆-cycloalkyl and C₁-C₆-haloalkyl;
Het is mono- or bicyclic heteroaryl selected from the group consisting of thienyl, thiazolyl, tetrazolyl, pyridyl and indolyl,
   where the heteroaryls mentioned may be partially or fully halogenated and/or may carry 1 to 2 radicals from the group consisting of nitro, C₁-C₄-alkyl, C₁-C₄-haloalkyl, hydroxyl, C₁-C₄-alkoxy, C₁-C₄-haloalkoxy, hydroxy-carbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)-amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)aminocarbonylamino;
R¹ and R² are hydrogen;
R³ is C₁-C₄-alkyl,
particularly preferably CH₃;
R⁴ is hydrogen, C₁-C₄-alkylcarbonyl, C₁-C₄-alkylaminocarbonyl, di-(C₁-C₄-alkyl)-aminocarbonyl, phenylaminocarbonyl, N—(C₁-C₄-alkyl)-N-(phenyl)aminocarbonyl, SO₂CH₃ or SO₂(C₆H₅); and
R⁵ is hydrogen.

Most preference is given to the compounds of the formula I.a.1 (corresponds to formula I where A=A1 where R⁷=H, R⁸=CF₃; Het=Het-1; R¹, R² and R⁵=H; R³=CH₃), in particular to the compounds of the formula I.a.1.1 to I.a.1.192 of Table 1, where the definitions of the variables A, Het and R¹ to R¹³ are of particular importance for the compounds according to the invention not only in combination with one another, but in each case also on their own.

TABLE 1

| | |
|---|---|
| | I.a.1 |
| 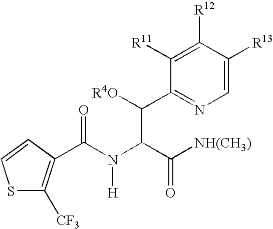 | |

| No. | R⁴ | R¹¹ | R¹² | R¹³ |
|---|---|---|---|---|
| I.a.1.1 | H | H | H | H |
| I.a.1.2 | H | H | H | F |
| I.a.1.3 | H | H | CH₃ | H |
| I.a.1.4 | H | H | CH₃ | F |
| I.a.1.5 | H | H | F | H |
| I.a.1.6 | H | H | F | F |
| I.a.1.7 | H | H | Cl | H |
| I.a.1.8 | H | H | Cl | F |
| I.a.1.9 | H | CH₃ | H | H |
| I.a.1.10 | H | CH₃ | H | F |
| I.a.1.11 | H | CH₃ | CH₃ | H |
| I.a.1.12 | H | CH₃ | CH₃ | F |
| I.a.1.13 | H | CH₃ | F | H |
| I.a.1.14 | H | CH₃ | F | F |
| I.a.1.15 | H | CH₃ | Cl | H |
| I.a.1.16 | H | CH₃ | Cl | F |
| I.a.1.17 | H | F | H | H |
| I.a.1.18 | H | F | H | F |
| I.a.1.19 | H | F | CH₃ | H |
| I.a.1.20 | H | F | CH₃ | F |
| I.a.1.21 | H | F | F | H |
| I.a.1.22 | H | F | F | F |
| I.a.1.23 | H | F | Cl | H |
| I.a.1.24 | H | F | Cl | F |
| I.a.1.25 | H | Cl | H | H |
| I.a.1.26 | H | Cl | H | F |
| I.a.1.27 | H | Cl | CH₃ | H |
| I.a.1.28 | H | Cl | CH₃ | F |
| I.a.1.29 | H | Cl | F | H |
| I.a.1.30 | H | Cl | F | F |
| I.a.1.31 | H | Cl | Cl | H |
| I.a.1.32 | H | Cl | Cl | F |
| I.a.1.33 | C(O)CH₃ | H | H | H |
| I.a.1.34 | C(O)CH₃ | H | H | F |
| I.a.1.35 | C(O)CH₃ | H | CH₃ | H |
| I.a.1.36 | C(O)CH₃ | H | CH₃ | F |
| I.a.1.37 | C(O)CH₃ | H | F | H |
| I.a.1.38 | C(O)CH₃ | H | F | F |
| I.a.1.39 | C(O)CH₃ | H | Cl | H |
| I.a.1.40 | C(O)CH₃ | H | Cl | F |
| I.a.1.41 | C(O)CH₃ | CH₃ | H | H |
| I.a.1.42 | C(O)CH₃ | CH₃ | H | F |
| I.a.1.43 | C(O)CH₃ | CH₃ | CH₃ | H |
| I.a.1.44 | C(O)CH₃ | CH₃ | CH₃ | F |
| I.a.1.45 | C(O)CH₃ | CH₃ | F | H |
| I.a.1.46 | C(O)CH₃ | CH₃ | F | F |
| I.a.1.47 | C(O)CH₃ | CH₃ | Cl | H |
| I.a.1.48 | C(O)CH₃ | CH₃ | Cl | F |
| I.a.1.49 | C(O)CH₃ | F | H | H |
| I.a.1.50 | C(O)CH₃ | F | H | F |
| I.a.1.51 | C(O)CH₃ | F | CH₃ | H |
| I.a.1.52 | C(O)CH₃ | F | CH₃ | F |
| I.a.1.53 | C(O)CH₃ | F | F | H |
| I.a.1.54 | C(O)CH₃ | F | F | F |
| I.a.1.55 | C(O)CH₃ | F | Cl | H |
| I.a.1.56 | C(O)CH₃ | F | Cl | F |
| I.a.1.57 | C(O)CH₃ | Cl | H | H |
| I.a.1.58 | C(O)CH₃ | Cl | H | F |
| I.a.1.59 | C(O)CH₃ | Cl | CH₃ | H |
| I.a.1.60 | C(O)CH₃ | Cl | CH₃ | F |
| I.a.1.61 | C(O)CH₃ | Cl | F | H |
| I.a.1.62 | C(O)CH₃ | Cl | F | F |
| I.a.1.63 | C(O)CH₃ | Cl | Cl | H |
| I.a.1.64 | C(O)CH₃ | Cl | Cl | F |
| I.a.1.65 | C(O)tertC₄H₉ | H | H | H |
| I.a.1.66 | C(O)tertC₄H₉ | H | H | F |
| I.a.1.67 | C(O)tertC₄H₉ | H | CH₃ | H |
| I.a.1.68 | C(O)tertC₄H₉ | H | CH₃ | F |
| I.a.1.69 | C(O)tertC₄H₉ | H | F | H |
| I.a.1.70 | C(O)tertC₄H₉ | H | F | F |
| I.a.1.71 | C(O)tertC₄H₉ | H | Cl | H |
| I.a.1.72 | C(O)tertC₄H₉ | H | Cl | F |
| I.a.1.73 | C(O)tertC₄H₉ | CH₃ | H | H |
| I.a.1.74 | C(O)tertC₄H₉ | CH₃ | H | F |
| I.a.1.75 | C(O)tertC₄H₉ | CH₃ | CH₃ | H |
| I.a.1.76 | C(O)tertC₄H₉ | CH₃ | CH₃ | F |
| I.a.1.77 | C(O)tertC₄H₉ | CH₃ | F | H |
| I.a.1.78 | C(O)tertC₄H₉ | CH₃ | F | F |
| I.a.1.79 | C(O)tertC₄H₉ | CH₃ | Cl | H |
| I.a.1.80 | C(O)tertC₄H₉ | CH₃ | Cl | F |
| I.a.1.81 | C(O)tertC₄H₉ | F | H | H |
| I.a.1.82 | C(O)tertC₄H₉ | F | H | F |
| I.a.1.83 | C(O)tertC₄H₉ | F | CH₃ | H |
| I.a.1.84 | C(O)tertC₄H₉ | F | CH₃ | F |
| I.a.1.85 | C(O)tertC₄H₉ | F | F | H |
| I.a.1.86 | C(O)tertC₄H₉ | F | F | F |
| I.a.1.87 | C(O)tertC₄H₉ | F | Cl | H |
| I.a.1.88 | C(O)tertC₄H₉ | F | Cl | F |
| I.a.1.89 | C(O)tertC₄H₉ | Cl | H | H |
| I.a.1.90 | C(O)tertC₄H₉ | Cl | H | F |
| I.a.1.91 | C(O)tertC₄H₉ | Cl | CH₃ | H |
| I.a.1.92 | C(O)tertC₄H₉ | Cl | CH₃ | F |
| I.a.1.93 | C(O)tertC₄H₉ | Cl | F | H |
| I.a.1.94 | C(O)tertC₄H₉ | Cl | F | F |
| I.a.1.95 | C(O)tertC₄H₉ | Cl | Cl | H |
| I.a.1.96 | C(O)tertC₄H₉ | Cl | Cl | F |
| I.a.1.97 | C(O)N(CH₃)₂ | H | H | H |
| I.a.1.98 | C(O)N(CH₃)₂ | H | H | F |
| I.a.1.99 | C(O)N(CH₃)₂ | H | CH₃ | H |
| I.a.1.100 | C(O)N(CH₃)₂ | H | CH₃ | F |
| I.a.1.101 | C(O)N(CH₃)₂ | H | F | H |
| I.a.1.102 | C(O)N(CH₃)₂ | H | F | F |
| I.a.1.103 | C(O)N(CH₃)₂ | H | Cl | H |
| I.a.1.104 | C(O)N(CH₃)₂ | H | Cl | F |
| I.a.1.105 | C(O)N(CH₃)₂ | CH₃ | H | H |
| I.a.1.106 | C(O)N(CH₃)₂ | CH₃ | H | F |
| I.a.1.107 | C(O)N(CH₃)₂ | CH₃ | CH₃ | H |
| I.a.1.108 | C(Q)N(CH₃)₂ | CH₃ | CH₃ | F |
| I.a.1.109 | C(O)N(CH₃)₂ | CH₃ | F | H |
| I.a.1.110 | O(O)N(CH₃)₂ | CH₃ | F | F |
| I.a.1.111 | C(O)N(CH₃)₂ | CH₃ | Cl | H |
| I.a.1.112 | C(O)N(CH₃)₂ | CH₃ | Cl | F |
| I.a.1.113 | C(O)N(CH₃)₂ | F | H | H |
| I.a.1.114 | C(O)N(CH₃)₂ | F | H | F |
| I.a.1.115 | C(O)N(CH₃)₂ | F | CH₃ | H |
| I.a.1.116 | C(O)N(CH₃)₂ | F | CH₃ | F |
| I.a.1.117 | C(O)N(CH₃)₂ | F | F | H |
| I.a.1.118 | C(O)N(CH₃)₂ | F | F | F |
| I.a.1.119 | C(O)N(CH₃)₂ | F | Cl | H |
| I.a.1.120 | C(O)N(CH₃)₂ | F | Cl | F |
| I.a.1.121 | C(O)N(CH₃)₂ | Cl | H | H |
| I.a.1.122 | C(O)N(CH₃)₂ | Cl | H | F |
| I.a.1.123 | C(O)N(CH₃)₂ | Cl | CH₃ | H |
| I.a.1.124 | C(O)N(CH₃)₂ | Cl | CH₃ | F |
| I.a.1.125 | C(O)N(CH₃)₂ | Cl | F | H |
| I.a.1.126 | C(O)N(CH₃)₂ | Cl | F | F |
| I.a.1.127 | C(O)N(CH₃)₂ | Cl | Cl | H |
| I.a.1.128 | C(O)N(CH₃)₂ | Cl | Cl | F |
| I.a.1.129 | C(O)N(CH₃)(C₆H₅) | H | H | H |
| I.a.1.130 | C(O)N(CH₃)(C₆H₅) | H | H | F |
| I.a.1.131 | C(O)N(CH₃)(C₆H₅) | H | CH₃ | H |
| I.a.1.132 | C(O)N(CH₃)(C₆H₅) | H | CH₃ | F |
| I.a.1.133 | C(O)N(CH₃)(C₆H₅) | H | F | H |
| I.a.1.134 | C(O)N(CH₃)(C₆H₅) | H | F | F |
| I.a.1.135 | C(O)N(CH₃)(C₆H₅) | H | Cl | H |
| I.a.1.136 | C(O)N(CH₃)(C₆H₅) | H | Cl | F |
| I.a.1.137 | C(O)N(CH₃)(C₆H₅) | CH₃ | H | H |
| I.a.1.138 | C(O)N(CH₃)(C₆H₅) | CH₃ | H | F |
| I.a.1.139 | C(O)N(CH₃)(C₆H₅) | CH₃ | CH₃ | H |
| I.a.1.140 | C(O)N(CH₃)(C₆H₅) | CH₃ | CH₃ | F |
| I.a.1.141 | C(O)N(CH₃)(C₆H₅) | CH₃ | F | H |
| I.a.1.142 | C(O)N(CH₃)(C₆H₅) | CH₃ | F | F |
| I.a.1.143 | C(O)N(CH₃)(C₆H₅) | CH₃ | Cl | H |
| I.a.1.144 | C(O)N(CH₃)(C₆H₅) | CH₃ | Cl | F |
| I.a.1.145 | C(O)N(CH₃)(C₆H₅) | F | H | H |
| I.a.1.146 | C(O)N(CH₃)(C₆H₅) | F | H | F |
| I.a.1.147 | C(O)N(CH₃)(C₆H₅) | F | CH₃ | H |
| I.a.1.148 | C(O)N(CH₃)(C₆H₅) | F | CH₃ | F |
| I.a.1.149 | C(O)N(CH₃)(C₆H₅) | F | F | H |
| I.a.1.150 | C(O)N(CH₃)(C₆H₅) | F | F | F |
| I.a.1.151 | C(O)N(CH₃)(C₆H₅) | F | Cl | H |
| I.a.1.152 | C(O)N(CH₃)(C₆H₅) | F | Cl | F |
| I.a.1.153 | C(O)N(CH₃)(C₆H₅) | Cl | H | H |
| I.a.1.154 | C(O)N(CH₃)(C₆H₅) | Cl | H | F |
| I.a.1.155 | C(O)N(CH₃)(C₆H₅) | Cl | CH₃ | H |
| I.a.1.156 | C(O)N(CH₃)(C₆H₅) | Cl | CH₃ | F |
| I.a.1.157 | C(O)N(CH₃)(C₆H₅) | Cl | F | H |
| I.a.1.158 | C(O)N(CH₃)(C₆H₅) | Cl | F | F |
| I.a.1.159 | C(O)N(CH₃)(C₆H₅) | Cl | Cl | H |
| I.a.1.160 | C(O)N(CH₃)(C₆H₅) | Cl | Cl | F |
| I.a.1.161 | SO₂CH₃ | H | H | H |
| I.a.1.162 | SO₂CH₃ | H | H | F |
| I.a.1.163 | SO₂CH₃ | H | CH₃ | H |
| I.a.1.164 | SO₂CH₃ | H | CH₃ | F |
| I.a.1.165 | SO₂CH₃ | H | F | H |
| I.a.1.166 | SO₂CH₃ | H | F | F |
| I.a.1.167 | SO₂CH₃ | H | Cl | H |
| I.a.1.168 | SO₂CH₃ | H | Cl | F |
| I.a.1.169 | SO₂CH₃ | CH₃ | H | H |
| I.a.1.170 | SO₂CH₃ | CH₃ | H | F |
| I.a.1.171 | SO₂CH₃ | CH₃ | CH₃ | H |
| I.a.1.172 | SO₂CH₃ | CH₃ | CH₃ | F |
| I.a.1.173 | SO₂CH₃ | CH₃ | F | H |
| I.a.1.174 | SO₂CH₃ | CH₃ | F | F |
| I.a.1.175 | SO₂CH₃ | CH₃ | Cl | H |
| I.a.1.176 | SO₂CH₃ | CH₃ | Cl | F |
| I.a.1.177 | SO₂CH₃ | F | H | H |
| I.a.1.178 | SO₂CH₃ | F | H | F |
| I.a.1.179 | SO₂CH₃ | F | CH₃ | H |
| I.a.1.180 | SO₂CH₃ | F | CH₃ | F |
| I.a.1.181 | SO₂CH₃ | F | F | H |
| I.a.1.182 | SO₂CH₃ | F | F | F |
| I.a.1.183 | SO₂CH₃ | F | Cl | H |
| I.a.1.184 | SO₂CH₃ | F | Cl | F |
| I.a.1.185 | SO₂CH₃ | Cl | H | H |
| I.a.1.186 | SO₂CH₃ | Cl | H | F |
| I.a.1.187 | SO₂CH₃ | Cl | CH₃ | H |
| I.a.1.188 | SO₂CH₃ | Cl | CH₃ | F |
| I.a.1.189 | SO₂CH₃ | Cl | F | H |
| I.a.1.190 | SO₂CH₃ | Cl | F | F |
| I.a.1.191 | SO₂CH₃ | Cl | Cl | H |
| I.a.1.192 | SO₂CH₃ | Cl | Cl | F |

Most preference is likewise given to the compounds of the formula I.a.2, in particular to the compounds of the formulae I.a.2.1 to I.a.2.192, which differ from the corresponding compounds of the formulae I.a.1.1 to I.a.1.192 in that A is A1 where R⁷=CH₃ and R⁸=CF₃.

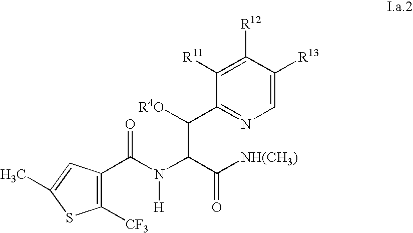

Most preference is likewise given to the compounds of the formula I.a.3, in particular to the compounds of the formulae I.a.3.1 to I.a.3.192, which differ from the corresponding compounds of the formulae I.a.1.1 to I.a.1.192 in that A is A2 where R⁷=H and R⁸=CF₃.

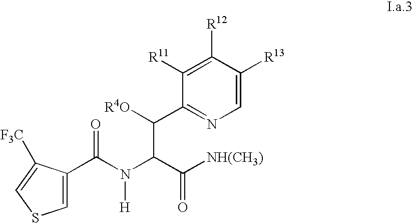

Most preference is likewise given to the compounds of the formula I.a.4, in particular to the compounds of the formulae I.a.4.1 to I.a.4.192, which differ from the corresponding compounds of the formulae I.a.1.1 to I.a.1.192 in that A is A3 where R⁷=H and R⁸=CF₃.

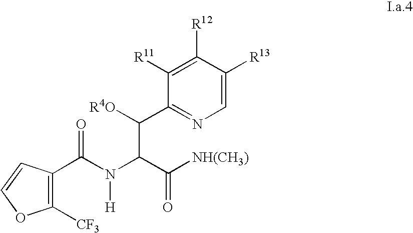

Most preference is likewise given to the compounds of the formula I.a.5, in particular to the compounds of the formulae I.a.5.1 to I.a.5.192, which differ from the corresponding compounds of the formulae I.a.1.1 to I.a.1.192 in that A is A3 where R⁷=CH₃ and R⁸=CF₃

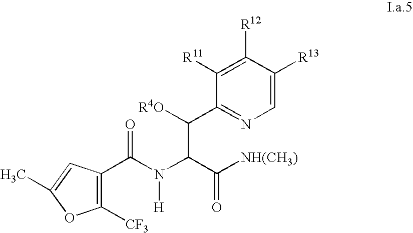

Most preference is likewise given to the compounds of the formula I.a.6, in particular to the compounds of the formulae I.a.6.1 to I.a.6.192, which differ from the corresponding compounds of the formulae I.a.1.1 to I.a.1.192 in that A is A4 where R⁷=H and R⁸=CF₃.

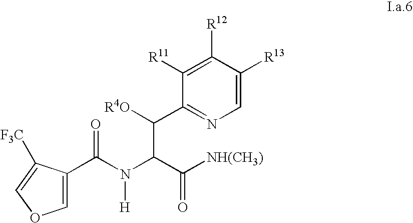

Most preference is likewise given to the compounds of the formula I.a.7, in particular to the compounds of the formulae I.a.7.1 to I.a.7.192, which differ from the corresponding compounds of the formulae I.a.1.1 to I.a.1.192 in that A is A5 where R⁷=H, R⁸=CF₃ and R⁹=H.

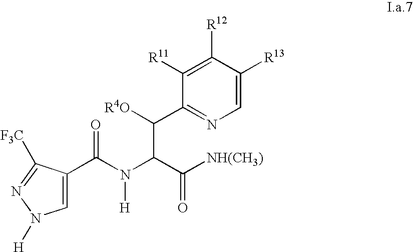

Most preference is likewise given to the compounds of the formula I.a.8, in particular to the compounds of the formulae I.a.8.1 to I.a.8.192, which differ from the corresponding compounds of the formulae I.a.1.1 to I.a.1.192 in that A is A5 where R⁷=CH₃, R⁸=CF₃ and R⁹=H.

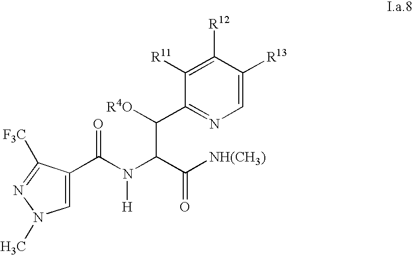

Most preference is likewise given to the compounds of the formula I.a.9, in particular to the compounds of the formulae I.a.9.1 to I.a.9.192, which differ from the corresponding compounds of the formulae I.a.1.1 to I.a.1.192 in that A is A8 where R⁷=H and R⁸=CF₃.

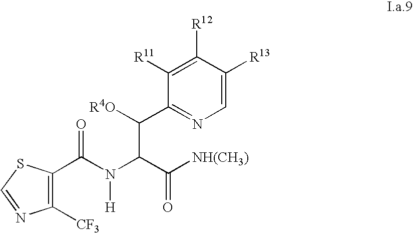

Most preference is likewise given to the compounds of the formula I.a.10, in particular to the compounds of the formulae I.a.10.1 to I.a.10.192, which differ from the corresponding compounds of the formulae I.a.1.1 to I.a.1.192 in that A is A8 where R⁷=CH₃ and R⁸=CF₃.

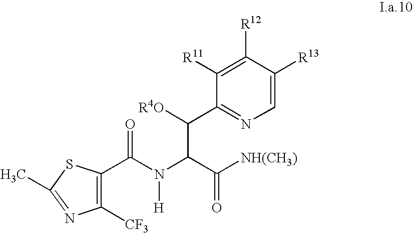

Most preference is likewise given to the compounds of the formula I.b.1, in particular to the compounds of the formulae I.b.1.1 to I.b.1.192, which differ from the corresponding compounds of the formulae I.a.1.1 to I.a.1.192 in that Het is Het-2.

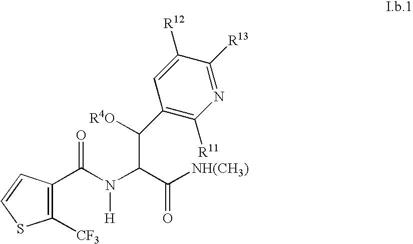

Most preference is likewise given to the compounds of the formula I.b.2, in particular to the compounds of the formulae I.b.2.1 to I.b.2.192, which differ from the corresponding compounds of the formulae I.a.1.1 to I.a.1.192 in that A is A1 where R⁷=CH₃ and R⁸=CF₃ and Het is Het-2.

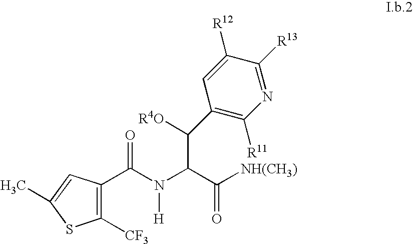

Most preference is likewise given to the compounds of the formula I.b.3, in particular to the compounds of the formulae I.b.3.1 to I.b.3.192, which differ from the corresponding compounds of the formulae I.a.1.1 to I.a.1.192 in that A is A2 where R⁷=H and R⁸=CF₃ and Het is Het-2.

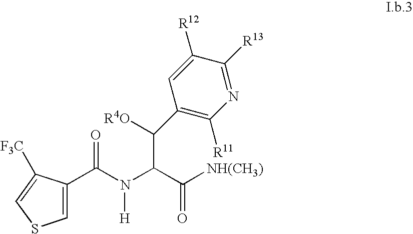

Most preference is likewise given to the compounds of the formula I.b.4, in particular to the compounds of the formulae I.b.4.1 to I.b.4.192, which differ from the corresponding compounds of the formulae I.a.1.1 to I.a.1.192 in that A is A3 where R⁷=H and R⁸=CF₃ and Het is Het-2.

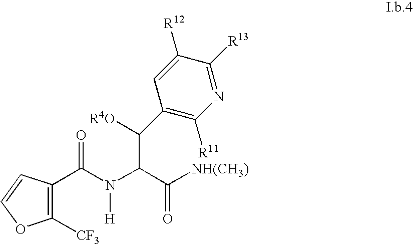

Most preference is likewise given to the compounds of the formula I.b.5, in particular to the compounds of the formulae I.b.5.1 to I.b.5.192, which differ from the corresponding compounds of the formulae I.a.1.1 to I.a.1.192 in that A is A3 where R⁷=CH₃ and R⁸=CF₃ and Het is Het-2.

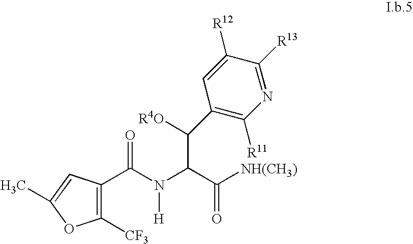

Most preference is likewise given to the compounds of the formula I.b.6, in particular to the compounds of the formulae I.b.6.1 to I.b.6.192, which differ from the corresponding compounds of the formulae I.a.1.1 to I.a.1.192 in that A is A4 where R⁷=H and R⁸=CF₃ and Het is Het-2.

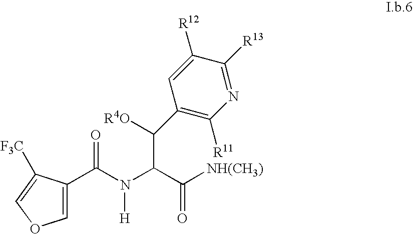

Most preference is likewise given to the compounds of the formula I.b.7, in particular to the compounds of the formulae I.b.7.1 to I.b.7.192, which differ from the corresponding compounds of the formulae I.a.1.1 to I.a.1.192 in that A is A5 where R⁷=H, R⁸=CF₃ and R⁹=H and Het is Het-2.

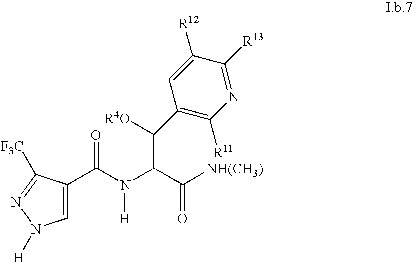

Most preference is likewise given to the compounds of the formula I.b.8, in particular to the compounds of the formulae I.b.8.1 to I.b.8.192, which differ from the corresponding compounds of the formulae I.a.1.1 to I.a.1.192 in that A is A5 where R⁷=CH₃, R⁸=CF₃ and R⁹=H and Het is Het-2.

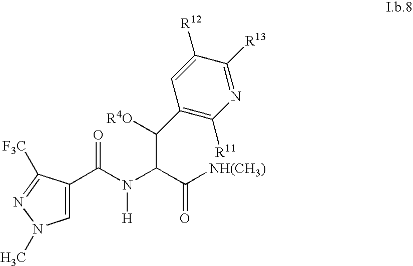

Most preference is likewise given to the compounds of the formula I.b.9, in particular to the compounds of the formulae I.b.9.1 to I.b.9.192, which differ from the corresponding compounds of the formulae I.a.1.1 to I.a.1.192 in that A is A8 where R⁷=H and R⁸=CF₃ and Het is Het-2.

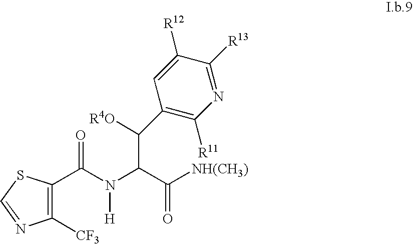

Most preference is likewise given to the compounds of the formula I.b.10, in particular to the compounds of the formulae I.b.10.1 to I.b.10.192, which differ from the corresponding compounds of the formulae I.a.1.1 to I.a.1.192 in that A is A8 where R⁷=CH₃ and R⁸=CF₃ and Het is Het-2.

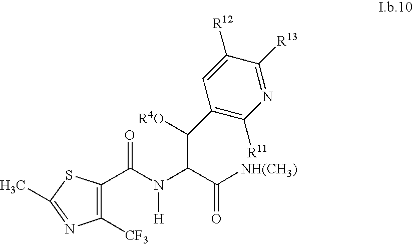

Most preference is likewise given to the compounds of the formula I.c.1, in particular to the compounds of the formulae I.c.1.1 to I.c.1.192, which differ from the corresponding compounds of the formulae I.a.1.1 to I.a.1.192 in that Het is Het-3.

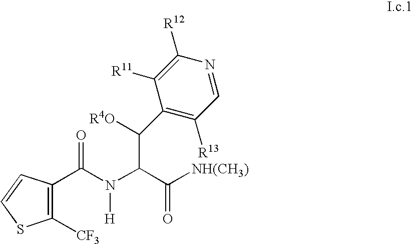

Most preference is likewise given to the compounds of the formula I.c.2, in particular to the compounds of the formulae I.c.2.1 to I.c.2.192, which differ from the corresponding compounds of the formulae I.a.1.1 to I.a.1.192 in that A is A1 where R⁷=CH₃ and R⁸=CF₃ and Het is Het-3.

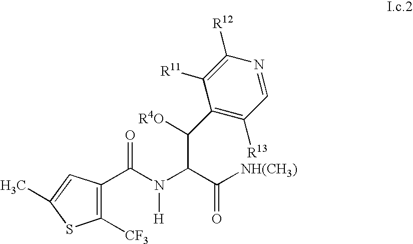

Most preference is likewise given to the compounds of the formula I.c.3, in particular to the compounds of the formulae I.c.3.1 to I.c.3.192, which differ from the corresponding compounds of the formulae I.a.1.1 to I.a.1.192 in that A is A2 where R⁷=H and R⁸=CF₃ and Het is Het-3.

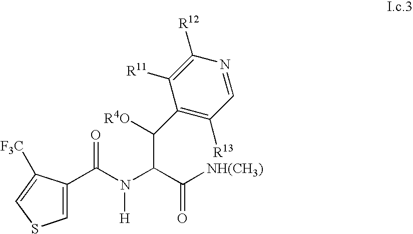

Most preference is likewise given to the compounds of the formula I.c.4, in particular to the compounds of the formulae I.c.4.1 to I.c.4.192, which differ from the corresponding compounds of the formulae I.a.1.1 to I.a.1.192 in that A is A3 where R⁷=H and R⁸=CF₃ and Het is Het-3.

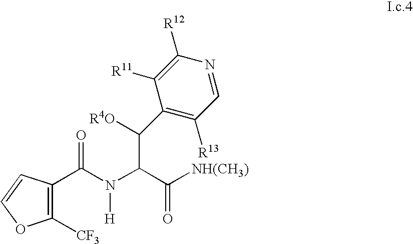

Most preference is likewise given to the compounds of the formula I.c.5, in particular to the compounds of the formulae I.c.5.1 to I.c.5.192, which differ from the corresponding compounds of the formulae I.a.1.1 to I.a.1.192 in that A is A3 where R⁷=CH₃ and R⁸=CF₃ and Het is Het-3.

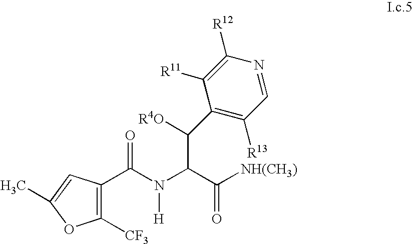

Most preference is likewise given to the compounds of the formula I.c.6, in particular to the compounds of the formulae I.c.6.1 to I.c.6.192, which differ from the corresponding compounds of the formulae I.a.1.1 to I.a.1.192 in that A is A4 where R⁷=H and R⁸=CF₃ and Het is Het-3.

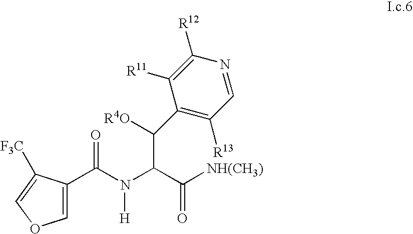

Most preference is likewise given to the compounds of the formula I.c.7, in particular to the compounds of the formulae I.c.7.1 to I.c.7.192, which differ from the corresponding compounds of the formulae I.a.1.1 to I.a.1.192 in that A is A5 where R⁷=H, R=CF₃ and R⁹=H and Het is Het-3.

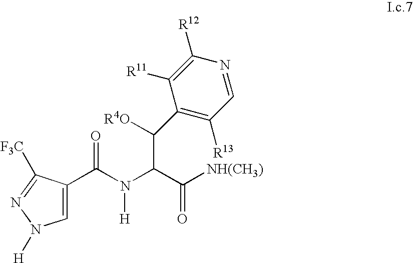

Most preference is likewise given to the compounds of the formula I.c.8, in particular to the compounds of the formulae I.c.8.1 to I.c.8.192, which differ from the corresponding compounds of the formulae I.a.1.1 to I.a.1.192 in that A is A5 where R⁷=CH₃, R⁸=CF₃ and R⁹=H and Het is Het-3.

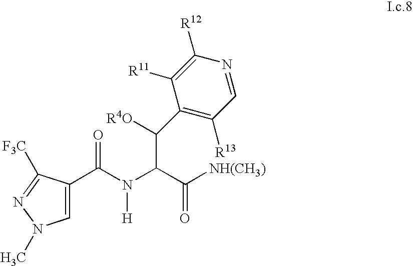

Most preference is likewise given to the compounds of the formula I.c.9, in particular to the compounds of the formulae I.c.9.1 to I.c.9.192, which differ from the corresponding compounds of the formulae I.a.1.1 to I.a.1.192 in that A is A8 where R⁷=H and R⁸=CF₃ and Het is Het-3.

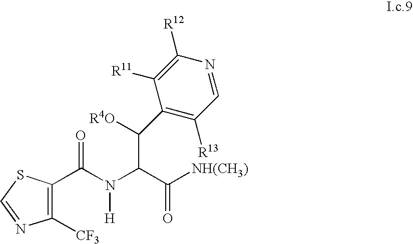

Most preference is likewise given to the compounds of the formula I.c.10, in particular to the compounds of the formulae I.c.10.1 to I.c.10.192, which differ from the corresponding compounds of the formulae I.a.1.1 to I.a.1.192 in that A is A8 where R⁷=CH₃ and R⁸=CF₃ and Het is Het-3.

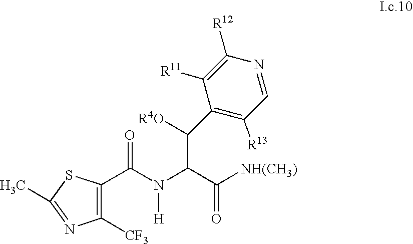

Most preference is likewise given to the compounds of the formula I.d.1, in particular to the compounds of the formulae I.d.1.1 to I.d.1.192, which differ from the corresponding compounds of the formulae I.a.1.1 to I.a.1.192 in that Het is Het-4.

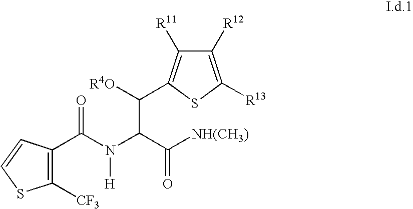

Most preference is likewise given to the compounds of the formula I.d.2, in particular to the compounds of the formulae I.d.2.1 to I.d.2.192, which differ from the corresponding compounds of the formulae I.a.1.1 to I.a.1.192 in that A is A1 where R⁷=CH₃ and R⁸=CF₃ and Het is Het-4.

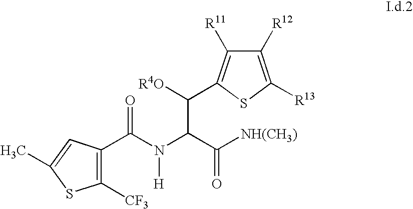

Most preference is likewise given to the compounds of the formula I.d.3, in particular to the compounds of the formulae I.d.3.1 to I.d.3.192, which differ from the corresponding compounds of the formulae I.a.1.1 to I.a.1.192 in that A is A2 where R⁷=H and R⁸=CF₃ and Het is Het-4.

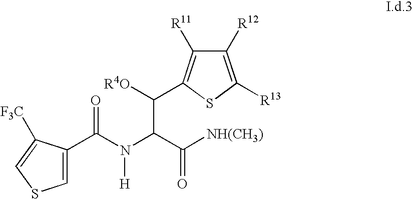

Most preference is likewise given to the compounds of the formula I.d.4, in particular to the compounds of the formulae I.d.4.1 to I.d.4.192, which differ from the corresponding compounds of the formulae I.a.1.1 to I.a.1.192 in that A is A3 where R⁷=H and R⁸=CF₃ and Het is Het4.

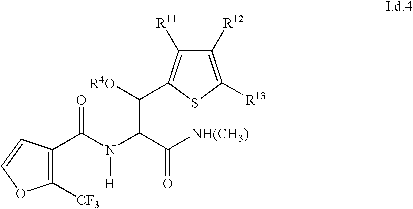

Most preference is likewise given to the compounds of the formula I.d.5, in particular to the compounds of the formulae I.d.5.1 to I.d.5.192, which differ from the corresponding compounds of the formulae I.a.1.1 to I.a.1.192 in that A is A3 where R⁷=CH₃ and R⁸=CF₃ and Het is Het4.

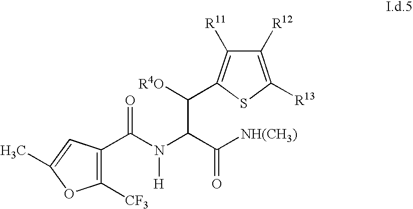

Most preference is likewise given to the compounds of the formula I.d.6, in particular to the compounds of the formulae I.d.6.1 to I.d.6.192, which differ from the corresponding compounds of the formulae I.a.1.1 to I.a.1.192 in that A is A4 where R⁷=H and R⁸=CF₃ and Het is Het4.

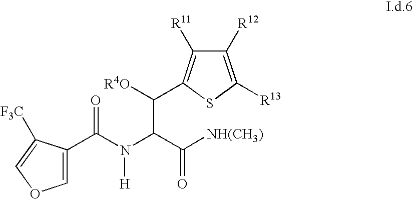

Most preference is likewise given to the compounds of the formula I.d.7, in particular to the compounds of the formulae I.d.7.1 to I.d.7.192, which differ from the corresponding compounds of the formulae I.a.1.1 to I.a.1.192 in that A is A5 where R⁷=H, R⁸=CF₃ and R⁹=H and Het is Het-4.

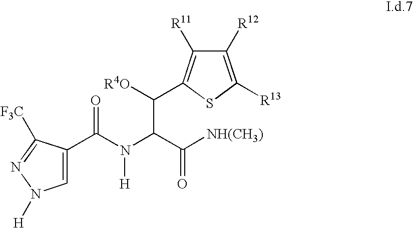

Most preference is likewise given to the compounds of the formula I.d.8, in particular to the compounds of the formulae I.d.8.1 to I.d.8.192, which differ from the corresponding compounds of the formulae I.a.1.1 to I.a.1.192 in that A is A5 where R⁷=CH₃, R³=CF₃ and R⁹=H and Het is Het-4.

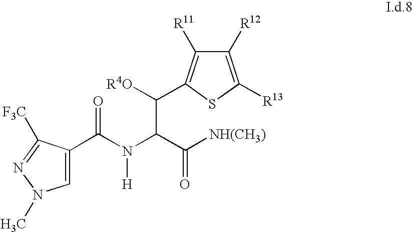

Most preference is likewise given to the compounds of the formula I.d.9, in particular to the compounds of the formulae I.d.9.1 to I.d.9.192, which differ from the corresponding compounds of the formulae I.a.1.1 to I.a.1.192 in that A is A8 where R⁷H and R⁸=CF₃ and Het is Het4.

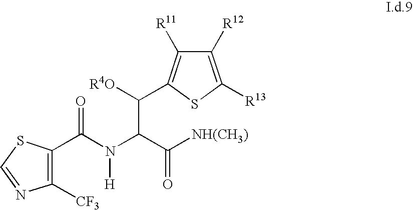

Most preference is likewise given to the compounds of the formula I.d.10, in particular to the compounds of the formulae I.d.10.1 to I.d.10.192, which differ from the corresponding compounds of the formulae I.a.1.1 to I.a.1.192 in that A is A8 where R⁷=CH₃ and R³=CF₃ and Het is Het-4.

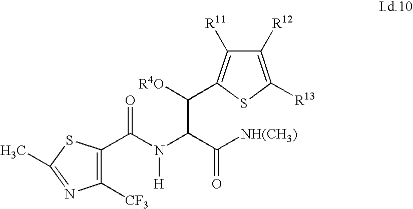

Most preference is likewise given to the compounds of the formula I.e. 1, in particular to the compounds of the formulae I.e.1.1 to I.e.1.192, which differ from the corresponding compounds of the formulae I.a.1.1 to I.a.1.192 in that Het is Het-5.

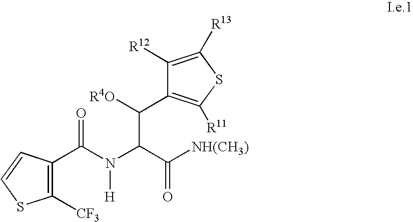

Most preference is likewise given to the compounds of the formula I.e.2, in particular to the compounds of the formulae I.e.2.1 to I.e.2.192, which differ from the corresponding compounds of the formulae I.a.1.1 to I.a.1.192 in that A is A1 where R⁷=CH₃ and R⁸=CF₃ and Het is Het-5.

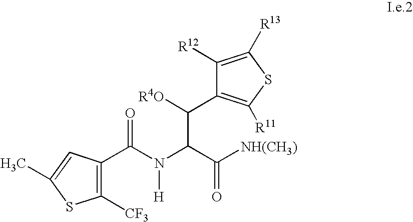

Most preference is likewise given to the compounds of the formula I.e.3, in particular to the compounds of the formulae I.e.3.1 to I.e.3.192, which differ from the corresponding compounds of the formulae I.a.1.1 to I.a.1.192 in that A is A2 where R⁷=H and R⁸=CF₃ and Het is Het-5.

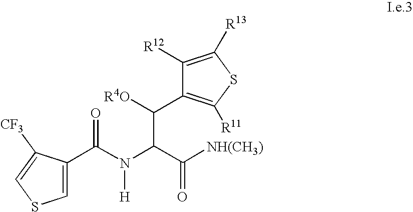

Most preference is likewise given to the compounds of the formula I.e.4, in particular to the compounds of the formulae I.e.4.1 to I.e.4.192, which differ from the corresponding compounds of the formulae I.a.1.1 to I.a.11.92 in that A is A3 where R⁷=H and R⁸=CF₃ and Het is Het-5.

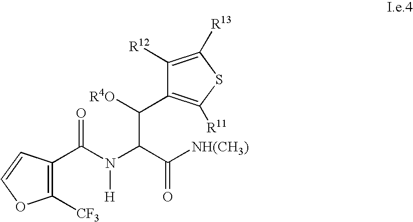

Most preference is likewise given to the compounds of the formula I.e.5, in particular to the compounds of the formulae I.e.5.1 to I.e.5.192, which differ from the corresponding compounds of the formulae I.a.1.1 to I.a.1.192 in that A is A3 where R⁷=CH₃ and R⁸=CF₃ and Het is Het-5.

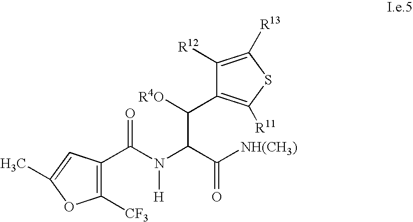

Most preference is likewise given to the compounds of the formula I.e.6, in particular to the compounds of the formulae I.e.6.1 to I.e.6.192, which differ from the corresponding compounds of the formulae I.a.1.1 to I.a.1.192 in that A is A4 where R⁷=H and R⁸=CF₃ and Het is Het-5.

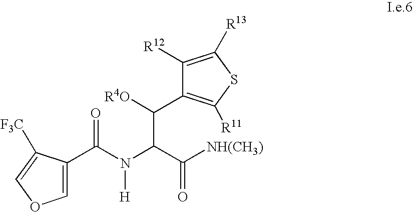

Most preference is likewise given to the compounds of the formula I.e.7, in particular to the compounds of the formulae I.e.7.1 to I.e.7.192, which differ from the corresponding compounds of the formulae I.a.1.1 to I.a.1.192 in that A is A5 where R⁷=H, R⁸=CF₃ and R⁹=H and Het is Het-5.

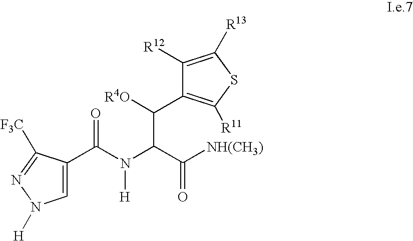

Most preference is likewise given to the compounds of the formula I.e.8, in particular to the compounds of the formulae I.e.8.1 to I.e.8.192, which differ from the corresponding compounds of the formulae I.a.1.1 to I.a.1.192 in that A is A5 where R⁷=CH₃, R⁸=CF₃ and R⁹=H and Het is Het-5.

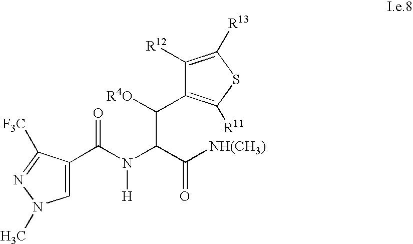

Most preference is likewise given to the compounds of the formula I.e.9, in particular to the compounds of the formulae I.e.9.1 to I.e.9.192, which differ from the corresponding compounds of the formulae I.a.1.1 to I.a.1.192 in that A is A8 where R⁷=H and R⁸=CF₃ and Het is Het-5.

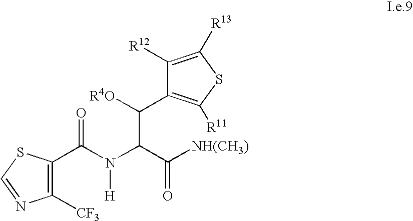

Most preference is likewise given to the compounds of the formula I.e.10, in particular to the compounds of the formulae I.e.10.1 to I.e.10.192, which differ from the corresponding compounds of the formulae I.a.1.1 to I.a.1.192 in that A is A8 where R⁷=CH₃ and R⁸=CF₃ and Het is Het-5.

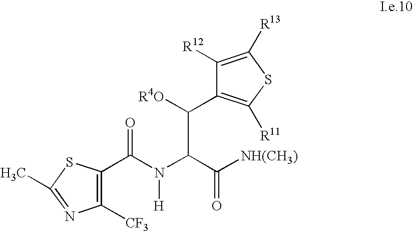

Most preference is likewise given to the compounds of the formula I.f.1, in particular to the compounds of the formulae I.f.1.1 to I.f.1.192, which differ from the corresponding compounds of the formulae I.a.1.1 to I.a.1.192 in that Het is Het-6.

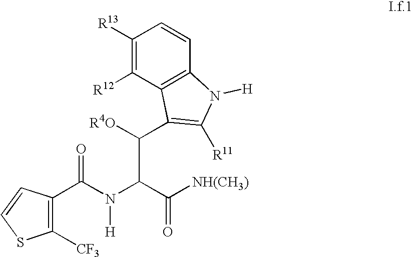

Most preference is likewise given to the compounds of the formula I.f.2, in particular to the compounds of the formulae I.f.2.1 to I.f.2.192, which differ from the corresponding compounds of the formulae I.a.1.1 to I.a.1.192 in that A is A1 where R⁷=CH₃ and R⁸=CF₃ and Het is Het-6.

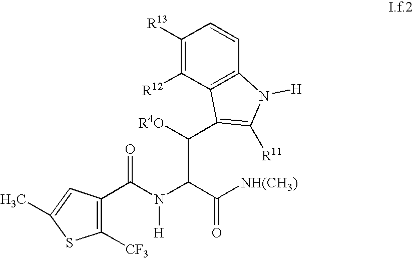

Most preference is likewise given to the compounds of the formula I.f.3, in particular to the compounds of the formulae I.f.3.1 to I.f.3.192, which differ from the corresponding compounds of the formulae I.a.1.1 to I.a.1.192 in that A is A2 where R⁷=H and R⁸=CF₃ and Het is Het-6.

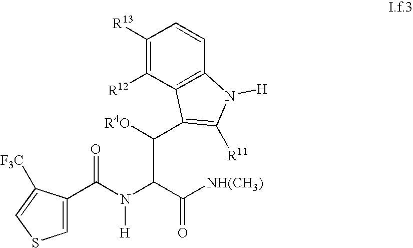

Most preference is likewise given to the compounds of the formula I.f.4, in particular to the compounds of the formulae I.f.4.1 to I.f.4.192, which differ from the corresponding compounds of the formulae I.a.1.1 to I.a.1.192 in that A is A3 where R⁷=H and R⁸=CF₃ and Het is Het-6.

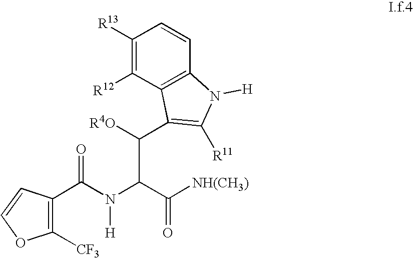

Most preference is likewise given to the compounds of the formula I.f.5, in particular to the compounds of the formulae I.f.5.1 to I.f.5.192, which differ from the corresponding compounds of the formulae I.a.1.1 to I.a.1.192 in that A is A3 where R⁷=CH₃ and R⁸=CF₃ and Het is Het-6.

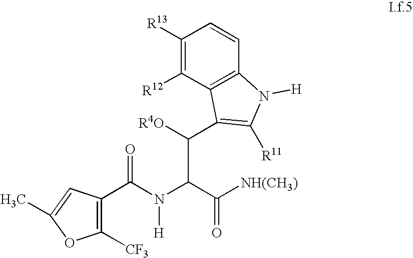

Most preference is likewise given to the compounds of the formula I.f.6, in particular to the compounds of the formulae I.f.6.1 to I.f.6.192, which differ from the corresponding compounds of the formulae I.a.1.1 to I.a.1.192 in that A is A4 where R⁷=H and R⁸=CF₃ and Het is Het-6.

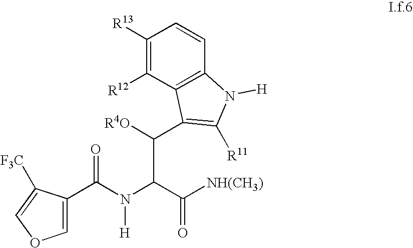

Most preference is likewise given to the compounds of the formula I.f.7, in particular to the compounds of the formulae I.f.7.1 to I.f.7.192, which differ from the corresponding compounds of the formulae I.a.1.1 to I.a.1.192 in that A is A5 where R⁷=H, R⁸=CF₃ and R⁹=H and Het is Het-6.

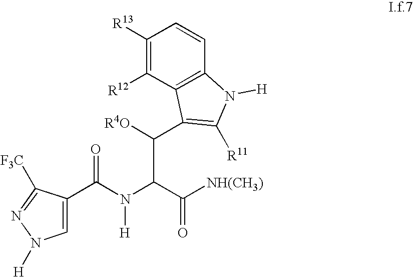

Most preference is likewise given to the compounds of the formula I.f.8, in particular to the compounds of the formulae I.f.8.1 to I.f.8.192, which differ from the corresponding compounds of the formulae I.a.1.1 to I.a.1.192 in that A is A5 where R⁷=CH₃, R⁸=CF₃ and R⁹=H and Het is Het-6.

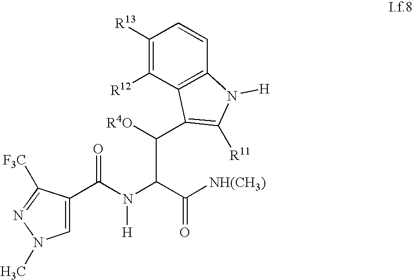

Most preference is likewise given to the compounds of the formula I.f.9, in particular to the compounds of the formulae I.f.9.1 to I.f.9.192, which differ from the corresponding compounds of the formulae I.a.1.1 to I.a.1.192 in that A is A8 where R⁷=H and R⁸=CF₃ and Het is Het-6.

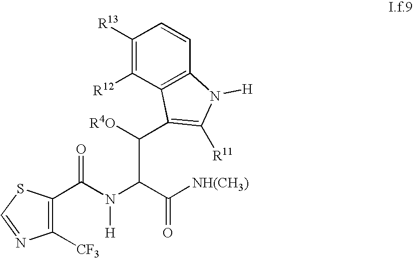

Most preference is likewise given to the compounds of the formula I.f.10, in particular to the compounds of the formulae I.f.10.1 to I.f.10.192, which differ from the corresponding compounds of the formulae I.a.1.1 to I.a.1.192 in that A is A8 where R⁷=CH₃ and R⁸=CF₃ and Het is Het-6.

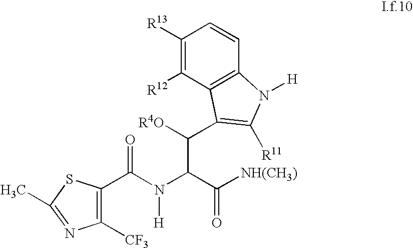

The benzoyl-substituted serineamides of the formula I can be obtained by different routes, for example by the following processes:

Process A

Serine derivatives of the formula V are initially reacted with heteroaryl acids/heteroaryl acid derivatives of the formula IV to give the corresponding heteroaroyl derivatives of the formula III which are then reacted with amines of the formula II to give the desired heteroaroyl-substituted serineamides of the formula I:

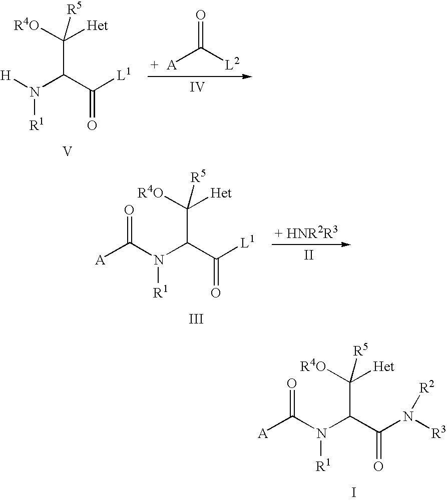

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₄-alkylsulfonyl, phosphoryl or isoureyl.

The reaction of the serine derivatives of the formula V with heteroaryl acids/heteroaryl acid derivatives of the formula IV where L² is hydroxyl to give heteroaroyl derivatives of the formula III is carried out in the presence of an activating reagent and a base, usually at temperatures of from 0° C. to the boiling point of the reaction mixture, preferably from 0° C. to 110° C., particularly preferably at room temperature, in an inert organic solvent [cf. Bergmann, E. D.; et al., J Chem Soc 1951, 2673; Zhdankin, V. V.; et al., Tetrahedron Lett. 2000, 41 (28), 5299-5302; Martin, S. F. et al., Tetrahedron Lett. 1998, 39 (12), 1517-1520; Jursic, B. S. et al., Synth Commun 2001, 31 (4), 555-564; Albrecht, M. et al., Synthesis 2001, (3), 468-472; Yadav, L. D. S. et al., Indian J. Chem B. 41(3), 593-595 (2002); Clark, J. E. et al., Synthesis (10), 891-894 (1991)].

Suitable activating reagents are condensing agents, such as, for example, polystyrene-bound dicyclohexylcarbodiimide, diisopropylcarbodiimide, carbonyldiimidazole, chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)phosphoryl chloride (BOPCI) or sulfonyl chlorides, such as methane-sulfonyl chloride, toluenesulfonyl chloride or benzenesulfonyl chloride.

Suitable solvents are aliphatic hydrocarbons, such as pentane, hexane, cyclohexane and mixtures of C₅-C₈-alkanes, aromatic hydrocarbons, such as benzene, toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran (THF), nitriles, such as acetonitrile and propionitrile, ketones, such as acetone, methyl ethyl ketone, diethyl ketone and tert-butyl methyl ketone, and also dimethyl sulfoxide, dimethylformamide (DMF), dimethyl-acetamide (DMA) and N-methylpyrrolidone (NMP), or else in water; particular preference is given to methylene chloride, THF and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methyl-morpholine and N-methylpiperidine, pyridine, substituted pyridines, such as collidine, lutidine and 4-dimethylaminopyridine, and also bicyclic amines. Particular preference is given to sodium hydroxide, triethylamine and pyridine.

The bases are generally employed in equimolar amounts. However, they can also be used in excess or, if appropriate, as solvent.

The starting materials are generally reacted with one another in equimolar amounts. It may be advantageous to use an excess of IV, based on V.

The reaction mixtures are worked up in a customary manner, for example by mixing with water, separating the phases and, if appropriate, chromatographic purification of the crude products. Some of the intermediates and end products are obtained in the form of viscous oils which are purified or freed from volatile components under reduced pressure and at moderately elevated temperatures. If the intermediates and end products are obtained as solids, purification can also be carried out by recrystallization or digestion.

The reaction of the serine derivatives of the formula V with heteroaryl acids/heteroaryl acid derivatives of the formula IV where L² is halogen, C₁-C₆-alkylcarbonyl, C₁-C₆-alkoxycarbonyl, C₁-C₄-alkylsulfonyl, phosphoryl or isoureyl to give heteroaroyl derivatives of the formula III is carried out in the presence of a base, usually at temperatures of from 0° C. to the boiling point of the reaction mixture, preferably at from 0° C. to 100° C., particularly preferably at room temperature, in an inert organic solvent [cf. Bergmann, E. D.; et al., J Chem Soc 1951, 2673; Zhdankin, V. V.; et al., Tetrahedron Lett. 2000, 41 (28), 5299-5302; Martin, S. F. et al., Tetrahedron Lett. 1998, 39 (12), 1517-1520; Jursic, B. S. et al., Synth Commun 2001, 31 (4), 555-564; Albrecht, M. et al., Synthesis 2001, (3), 468-472; Yadav, L. D. S. et al., Indian J. Chem B. 41(3), 593-595 (2002); Clark, J. E. et al., Synthesis (10), 891-894 (1991)].

Suitable solvents are aliphatic hydrocarbons, such as pentane, hexane, cyclohexane and mixtures of C₅-C₈-alkanes, aromatic hydrocarbons, such as benzene, toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran (THF), nitriles, such as acetonitrile and propionitrile, ketones, such as acetone, methyl ethyl ketone, diethyl ketone and tert-butyl methyl ketone, and also dimethyl sulfoxide, dimethylformamide (DMF), dimethylacetamide (DMA) and N-methylpyrrolidone (NMP), or else in water; particular preference is given to methylene chloride, THF and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methyl-morpholine and N-methylpiperidine, pyridine, substituted pyridines, such as collidine, lutidine and 4-dimethylaminopyridine, and also bicyclic amines. Particular preference is given to sodium hydroxide, triethylamine and pyridine.

The bases are generally employed in equimolar amounts. However, they can also be used in excess or, if appropriate, as solvent.

The starting materials are generally reacted with one another in equimolar amounts. It may be advantageous to use an excess of IV, based on V.

Work-up and isolation of the products can be carried out in a manner known per se.

It is, of course, also possible to initially react the serine derivatives of the formula V in an analogous manner with amines of the formula II to give the corresponding amides which are then reacted with heteroaryl acids/heteroaryl acid derivatives of the formula IV to give the desired heteroaroyl-substituted serineamides of the formula I.

The serine derivatives of the formula V (for example where L¹=hydroxyl or C₁-C₆-alkoxy) required for preparing the heteroaroyl derivatives of the formula III are known from the literature, even in enantiomerically and diastereomerically pure form, or they can be prepared in accordance with the literature cited:
by condensation of glycine enolate equivalents with heterocyclyl aldehydes or heterocyclyl ketones [Blaser, D. et al., Liebigs Ann. Chem. 10, 1067-1078 (1991); Seethaler, T. et al., Liebigs Ann. Chem. 1, 11-17 (1991); Weltenauer, G. et al., Gazz. Chim. Ital. 81, 162 (1951); Dalla Croce, P. et al., Heterocycles 52(3), 1337-1344 (2000); Van der Werf, A. W. et al., J. Chem. Soc. Chem. Commun. 100, 682-683 (1991); Caddick, S. et al., Tetrahedron 57 (30), 6615-6626 (2001); Owa, T. et al., Chem. Lett. 1, 83-86 (1988); Alker, D. et al., Tetrahedron 54 (22), 6089-6098 (1998); Rousseau, J. F. et al., J. Org. Chem. 63 (8), 2731-2737 (1998); Saeed, A. et al., Tetrahedron 48 (12), 2507-2514 (1992); Dong, L. et al., J. Org. Chem. 67 (14), 4759-4770 (2002)].
by aminohydroxylation of 3-heterocyclyl-substituted acrylic acid derivatives [Zhang, H. X. et al., Tetrahedron Asymmetr. 11(16), 3439-3447 (2000); Fokin, V. V. et al., Angew. Chem. Int. Edit. 40(18), 3455 (2001); Sugiyama, H. et al., Tetrahedron Lett. 43(19), 3489-3492 (2002); Bushey, M. L. et al., J. Org. Chem. 64(9), 2984-2985 (1999); Raatz, D. et al., Syntett (12), 1907-1910 (1999)].
by nucleophilic substitution of leaving groups in the 2-position of 3-heterocyclyl-3-hydroxypropionic acid derivatives [Owa, T. et al., Chem. Lett. (11), 1873-1874 (1988); Boger, D. L. et al., J. Org. Chem. 57(16), 4331-4333 (1992); Alcaide, B. et al., Tetrahedron Lett. 36(30), 5417-5420 (1995)].
by condensation of heterocyclyl aldehydes with nucleophiles with formation of oxazolines and subsequent hydrolysis [Evans, D. A. et al., Angew. Chem. Int. Edit. 40(10), 1884-1888 (2001); Ito, Y. et al., Tetrahedron Lett. 26(47), 5781-5784 (1985); Togni, A. et al., J. Organomet. Chem. 381(1), C21-5 (1990); Longmire, J. M. et al., Organometallics 17(20), 4374-4379 (1998); Suga, H. et al., J. Org. Chem. 58(26), 7397-7405 (1993)].
by oxidative cyclization of 2-acylamino-3-heterocyclylpropionic acid derivatives to give oxazolines and subsequent hydrolysis (JP10101655).
by hetero-Diels-Alder reactions of vinylimines with heterocyclyl aldehydes to give the tetrahydrooxazine and subsequent hydrolysis [Bongini, A. et al., Tetrahedron Asym. 12(3), 439-454 (2001)].

The heteroaroyl acids/heteroaryl acid derivatives of the formula IV required for preparing the heteroaroyl derivatives of the formula III are commercially available or can be prepared analogously to procedures known from the literature from the corresponding halide by a Grignard reaction [for example A. Mannschuk et al., Angew. Chem. 100, 299 (1988)].

The reaction of the heteroaroyl derivatives of the formula III where L¹=hydroxyl or salts thereof with amines of the formula II to give the desired heteroaroyl-substituted serineamides of the formula I is carried out in the presence of an activating reagent and, if appropriate, in the presence of a base, usually at temperatures of from 0° C. to the boiling point of the reaction mixture, preferably at from 0° C. to 100° C., particularly preferably at room temperature, in an inert organic solvent [cf. Perich, J. W., Johns, R. B., J. Org. Chem. 53 (17), 4103-4105 (1988); Somlai, C. et al., Synthesis (3), 285-287 (1992); Gupta, A. et al., J. Chem. Soc. Perkin Trans. 2, 1911 (1990); Guan et al., J. Comb. Chem. 2, 297 (2000)].

Suitable activating reagents are condensing agents, such as, for example, polystyrene-bound dicyclohexylcarbodiimide, diisopropylcarbodiimide, carbonyldiimidazole, chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)phosphoryl chloride (BOPCI) or sulfonyl chlorides, such as methane-sulfonyl chloride, toluenesulfonyl chloride or benzenesulfonyl chloride.

Suitable solvents are aliphatic hydrocarbons, such as pentane, hexane, cyclohexane and mixtures of C₅-C₈-alkanes, aromatic hydrocarbons, such as benzene, toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide (DMF), dimethylacetamide (DMA) and N-methylpyrrolidone (NMP), or else in water; particular preference is given to methylene chloride, THF, methanol, ethanol and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methyl-morpholine and N-methylpiperidine, pyridine, substituted pyridines, such as collidine, lutidine and 4-dimethylaminopyridine, and also bicyclic amines. Particular preference is given to sodium hydroxide, triethylamine, ethyldiisopropylamine, N-methylmorpholine and pyridine.

The bases are generally employed in catalytic amounts; however, they can also be employed in equimolar amounts, in excess or, if appropriate, as solvent.

The starting materials are generally reacted with one another in equimolar amounts. It may be advantageous to use an excess of II, based on Ill.

Work-up and isolation of the products can be carried out in a manner known per se.

The reaction of the heteroaroyl derivatives of the formula III where L¹=C₁-C₆-alkoxy with amines of the formula II to give the desired heteroaroyl-substituted serineamides of the formula I is usually carried out at temperatures of from 0° C. to the boiling point of the reaction mixture, preferably from 0° C. to 10° C., particularly preferably at room temperature, in an inert organic solvent, if appropriate in the presence of a base [cf. Kawahata, N. H. et al., Tetrahedron Lett. 43 (40), 7221-7223 (2002); Takahashi, K. et al., J. Org. Chem. 50 (18), 3414-3415 (1985); Lee, Y. et al., J. Am. Chem. Soc. 121 (36), 8407-8408 (1999)].

Suitable solvents are aliphatic hydrocarbons, such as pentane, hexane, cyclohexane and mixtures of C₅-C₈-alkanes, aromatic hydrocarbons, such as benzene, toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide (DMF), dimethylacetamide (DMA) and N-methylpyrrolidone (NMP), or else in water; particular preference is given to methylene chloride, THF, methanol, ethanol and water.

It is also possible to use mixtures of the solvents mentioned.

If appropriate, the reaction can be carried out in the presence of a base. Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methyl-morpholine and N-methylpiperidine, pyridine, substituted pyridines, such as collidine, lutidine and 4-dimethylaminopyridine, and also bicyclic amines. Particular preference is given to sodium hydroxide, triethylamine, ethyldiisopropylamine, N-methylmorpholine and pyridine.

The bases are generally employed in catalytic amounts; however, they can also be employed in equimolar amounts, in excess or, if appropriate, as solvent.

The starting materials are generally reacted with one another in equimolar amounts. It may be advantageous to use an excess of II, based on III.

Work-up and isolation of the products can be carried out in a manner known per se.

The amines of the formula II required for preparing the heteroaroyl-substituted serineamides of the formula I are commercially available.

Process B

Heteroaroyl derivatives of the formula III where R⁴=hydrogen can also be obtained by condensing acylated glycine derivatives of the formula VIII where the acyl group may be a cleavable protective group, such as benzyloxycarbonyl (cf. VIIIa where Σ=benzyl) or tert-butyloxycarbonyl (cf. VIIIa where Σ=tert-butyl), with heterocyclylcarbonyl compounds VII to give the corresponding aldol products VI. The protective group is then cleaved and the resulting serine derivative of the formula V where R⁴=hydrogen is acylated using heteroaryl acid derivatives of the formula IV.

Analogously, it is also possible to convert an acylated glycine derivative of the formula VIII where the acyl group is a substituted heteroaroyl radical (cf. VIIIb) in the presence of a base with a heterocyclylcarbonyl compound VII into the heteroaroyl derivative III where R⁴=hydrogen:

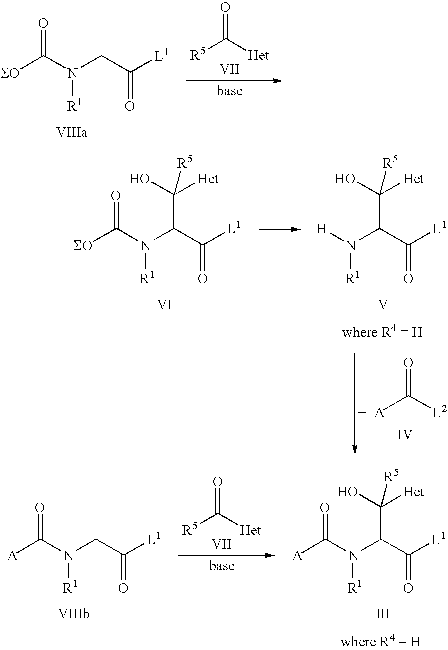

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₄-alkylsulfonyl, phosphoryl or isoureyl.

The reaction of the glycine derivatives VIII with heterocyclyl compounds VII to give the corresponding aldol product VI or heteroaroyl derivative III where R⁴=hydrogen is usually carried out at temperatures of from −100° C. to the boiling point of the reaction mixture, preferably at from −80° C. to 20° C., particularly preferably at from −80° C. to −20° C., in an inertorganic solvent in the presence of a base [cf. J.-F. Rousseau et al., J. Org. Chem. 63, 2731-2737 (1998)].

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, tert-butyl methyl ether, dioxane, anisole and tetrahydrofuran, and also dimethyl sulfoxide, dimethylformamide and dimethylacetamide, particularly preferably diethyl ether, dioxane and tetrahydrofuran.

It is also possible to use mixtures of the solvents mentioned.

Suitable bases are, in general, inorganic compounds, such as alkali metal and alkaline earth metal hydrides, such as lithium hydride, sodium hydride, potassium hydride and calcium hydride, alkali metal azides, such as lithium hexamethyldisilazide, organometallic compounds, in particular alkali metal alkyls, such as methyllithium, butyllithium and phenyllithium, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to sodium hydride, lithium hexamethyldisilazide and lithium diisopropylamide.

The bases are generally employed in equimolar amounts; however, they can also be used catalytically, in excess or, if appropriate, as solvents.

The starting materials are generally reacted with one another in equimolar amounts. It may be advantageous to employ an excess of base and/or heterocyclylcarbonyl compounds VII, based on the glycine derivatives VIII.

Work-up and isolation of the products can be carried out in the manner known per se. The glycine derivatives of the formula VIII required for preparing the compounds I are commercially available, known from the literature [for example H. Pessoa-Mahana et al., Synth. Comm. 32, 1437 (2002)] or can be prepared in accordance with the literature cited.

The protective group is cleaved off by methods known from the literature, giving serine derivatives of the formula V where R⁴=hydrogen [cf. J.-F. Rousseau et al., J. Org. Chem. 63, 2731-2737 (1998); J. M. Andres, Tetrahedron 56, 1523 (2000)]; in the case of Σ=benzyl by hydrogenolysis, preferably using hydrogen and Pd/C in methanol; in the case of Σ=tert-butyl using acid, preferably hydrochloric acid in dioxane.

The reaction of the serine derivatives V where R⁴=hydrogen with heteroaryl acids/heteroaryl acid derivatives IV to give heteroaroyl derivatives III where R⁴=hydrogen is usually carried out analogously to the reaction of the serine derivatives of the formula V with heteroaryl acids/heteroaryl acid derivatives of the formula IV to give heteroaroyl derivatives III mentioned in process A.

Analogously to process A, the heteroaroyl derivatives of the formula III where R⁴=hydrogen can then be reacted with amines of the formula II to give the desired heteroaroyl-substituted serineamides of the formula I where R⁴=hydrogen which can then be derivatized with compounds of the formula IX to give heteroaroyl-substituted serineamides of the formula I [cf., for example, Yokokawa, F. et al., Tetrahedron Lett. 42 (34), 5903-5908 (2001); Arrault, A. et al., Tetrahedron Lett. 43 (22), 4041-4044 (2002)].

It is also possible to initially derivatize the heteroaroyl derivatives of the formula III where R⁴=hydrogen with compounds of the formula IX to give further heteroaroyl derivatives of the formula III [cf., for example, Troast, D. et al., Org. Lett. 4 (6), 991-994 (2002); Ewing W. et al., Tetrahedron Lett., 30 (29), 3757-3760 (1989); Paulsen, H. et al., Liebigs Ann. Chem. 565 (1987)], followed by reaction with amines of the formula II analogously to process A, giving the desired heteroaroyl-substituted serineamides of the formula I:

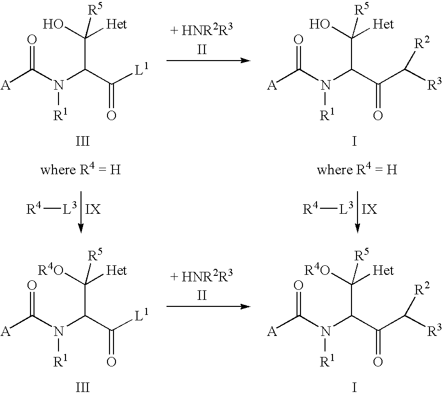

L¹ is a nucleophilically displaceable leaving group, for example hydroxyl or C₁-C₆-alkoxy.

L³ is a nucleophilically displaceable leaving group, for example halogen, hydroxyl, or C₁-C₆-alkoxy.

The reaction of the heteroaroyl derivatives of the formula III (where, if appropriate, R⁴=hydrogen) with amines of the formula II to give heteroaroyl-substituted serineamides of the formula I (where, if appropriate, R⁴=hydrogen) is usually carried out analogously to the reaction of the heteroaroyl derivatives of the formula III with amines of the formula II described in process A.

The reaction of the heteroaroyl derivatives of the formula III where R⁴=hydrogen or of the heteroaroyl-substituted serineamides of the formula I where R⁴=hydrogen with compounds of the formula IX to give heteroaroyl derivatives of the formula III or heteroaroyl-substituted serineamides of the formula I is usually carried out at temperatures of from 0° C. to 100° C., preferably from 10° C. to 50° C., in an inert organic solvent in the presence of a base [cf., for example, Troast, D. et al., Org. Lett. 4 (6), 991-994 (2002); Ewing W. et al., Tetrahedron Lett., 30 (29), 3757-3760 (1989); Paulsen, H. et al., Liebigs Ann. Chem. 565 (1987)].

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dichloromethane, tert-butyl methyl ether, dioxane and tetrahydrofuran.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to sodium hydroxide, sodium hydride and triethylamine.

The bases are generally employed in equimolar amounts; however, they can also be employed catalytically, in excess or, if appropriate, as solvents.

The starting materials are generally reacted with one another in equimolar amounts. It may be advantageous to use an excess of base and/or IX, based on III or 1.

Work-up and isolation of the products can be carried out in a manner known per se.

The required compounds of the formula VIII are commercially available.

Process C

Heteroaroyl derivatives of the formula III where R⁴=hydrogen can also be obtained by initially acylating aminomalonyl compounds of the formula X1 with heteroaryl acids/heteroaryl acid derivatives of the formula IV to give the corresponding N-acyl-aminomalonyl compounds of the formula X, followed by condensation with a heterocyclylcarbonyl compound of the formula VII with decarboxylation:

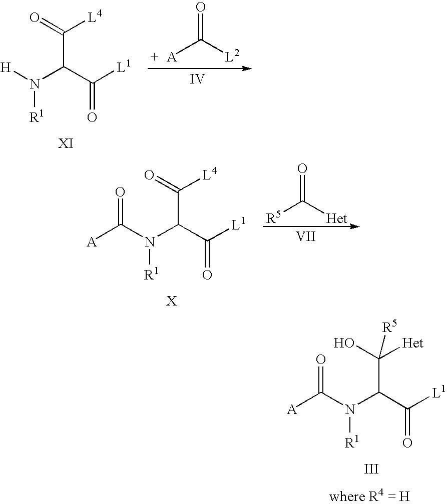

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₆-alkylsulfonyl, phosphoryl or isoureyl.

L⁴ is a nucleophilically displaceable leaving group, for example hydroxyl or C₁-C₆-alkoxy.

The acylation of the aminomalonyl compounds of the formula X1 with heteroaryl acids/heteroaryl acid derivatives of the formula IV to give the corresponding N-acyl-aminomalonyl compounds of the formula X is usually carried out analogously to the reaction, mentioned in process A, of the serine derivatives of the formula V with heteroaryl acids/heteroaryl acid derivatives of the formula IV to give the corresponding heteroaroyl derivatives of the formula III.

The reaction of the N-acylaminomalonyl compounds of the formula X with heterocyclylcarbonyl compounds of the formula VII give heteroaroyl derivatives of the formula III where R⁴=hydrogen is usually carried out at temperatures of from 0° C. to 100° C., preferably from 10° C. to 50° C., in an inert organic solvent in the presence of a base [cf., for example, U.S. Pat. No. 4,904,674; Hellmann, H. et al., Liebigs Ann. Chem. 631, 175-179 (1960)].

If L⁴ in the N-acylaminomalonyl compounds of the formula X is C₁-C₆-alkoxy, it is advantageous to initially convert L⁴ by ester hydrolysis [for example Hellmann, H. et al., Liebigs Ann. Chem. 631, 175-179 (1960)] into a hydroxyl group.

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably diethyl ether, dioxane and tetrahydrofuran.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to triethylamine and diisopropylethylamine.

The bases are generally employed in catalytic amounts; however, they can also be used in equimolar amounts, in excess or, if appropriate, as solvents.

The starting materials are generally reacted with one another in equimolar amounts. It may be advantageous to employ an excess of base, based on X.

Work-up and isolation of the products can be carried out in a manner known per se.

According to process A or B mentioned above, the resulting heteroaroyl derivatives of the formula III where R⁴=hydrogen can then be converted into the desired heteroaroyl-substituted serineamides of the formula I.

The required aminomalonyl compounds of the formula X1 are commercially available and/or known from the literature [for example U.S. Pat. No. 4,904,674; Hellmann, H. et al., Liebigs Ann. Chem. 631, 175-179 (1960)], or they can be prepared in accordance with the literature cited.

The required heterocyclic compounds of the formula VII are commercially available.

Process D

Heteroaroyl derivatives of the formula III where R⁴ and R⁵=hydrogen can also be obtained by initially reducing keto compounds of the formula XIII with heteroaryl acids/heteroaryl acid derivatives of the formula IV to give the corresponding N-acyl keto compounds of the formula XIII, followed by reduction of the keto group [Girard A, Tetrahedron Lett. 37 (44), 7967-7970 (1996); Nojori R., J. Am. Chem. Soc. 111 (25), 9134-9135 (1989); Schmidt U., Synthesis (12), 1248-1254 (1992); Bolhofer, A.; J. Am. Chem. Soc. 75, 4469 (1953)]:

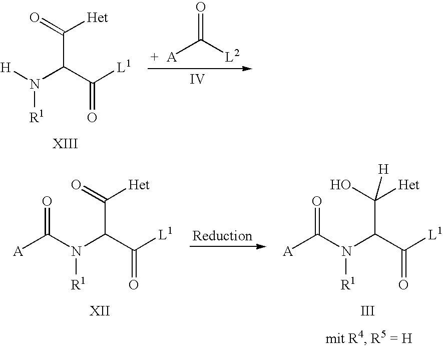

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₆-alkylsulfonyl, phosphoryl or isoureyl.

The acylation of the keto compounds of the formula XIII with heteroaryl acids/heteroaryl acid derivatives of the formula IV to give N-acyl keto compounds of the formula XII is usually carried out analogously to the reaction, mentioned in process A, of the serine derivatives of the formula V with heteroaryl acids/heteroaryl acid derivatives of the formula IV to give the corresponding heteroaroyl derivatives of the formula III.

The keto compounds of the formula XIII required for preparing the heteroaroyl derivatives of the formula III where R⁴ and R⁵=hydrogen are known from the literature [WO 02/083111; Boto, A. et al., Tetrahedron Letters 39 (44), 8167-8170 (1988); von Geldern, T. et al., J. of Med. Chem. 39(4), 957-967 (1996); Singh, J. et al., Tetrahedron Letters 34 (2), 211-214 (1993); ES 2021557; Maeda, S: et al., Chem. & Pharm. Bull. 32 (7), 2536-2543 (1984); Ito, S. et al., J. of Biol. Chem. 256 (15), 7834-4783 (1981); Vinograd, L. et al., Zhurnal Organicheskoi Khimii 16 (12), 2594-2599 (1980); Castro, A. et al., J. Org. Chem. 35 (8), 2815-2816 (1970); JP 02-172956; Suzuki, M. et al., J. Org. Chem. 38 (20), 3571-3575 (1973); Suzuki, M. et al, Synthetic Communications 2 (4), 237-242 (1972)] or can be prepared according to the literature cited.

The reduction of the N-acyl keto compounds of the formula XIII to heteroaroyl derivatives of the formula III where R⁴ and R⁵=hydrogen is usually carried out at temperatures of from 0° C. to 10° C., preferably from 20° C. to 80° C., in an inert organic solvent in the presence of a reducing agent.

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethyl formamide and dimethyl acetamide, particularly preferably toluene, methylene chloride or tert-butyl methyl ether.

It is also possible to use mixtures of the solvents mentioned.

Suitable reducing agents are, for example, sodium borohydride, zinc borohydride, sodium cyanoborohydride, lithium triethylborohydride (Superhydrid®), lithium tri-sec-butylborohydride (L-Selectrid®), lithium aluminum hydride or borane [cf., for example, WO 00/20424; Marchi, C. et al., Tetrahedron 58 (28), 5699 (2002); Blank, S. et al., Liebigs Ann. Chem. (8), 889-896 (1993); Kuwano, R. et al., J. Org. Chem. 63 (10), 3499-3503 (1998); Clariana, J. et al., Tetrahedron 55 (23), 7331-7344 (1999)].

Furthermore, the reduction can also be carried out in the presence of hydrogen and a catalyst. Suitable catalysts are, for example, [Ru(BlNAP)Cl₂] or Pd/C [cf. Noyori, R. et al., J. Am. Chem. Soc. 111 (25), 9134-9135 (1989); Bolhofer, A. et al., J. Am. Chem. Soc. 75, 4469 (1953)].

In addition, the reduction can also be carried out in the presence of a microorganism. The suitable microorganism is, for example, Saccharomyces rouxii [cf. Soukup, M. et al., Helv. Chim. Acta 70, 232 (1987)].

The N-acyl keto compounds of the formula XIII and the reducing agent in question are generally reacted with one another in equimolar amounts. It may be advantageous to employ an excess of reducing agent, based on XII.

Work-up and isolation of the products can be carried out in the manner known per se.

The resulting heteroaroyl derivatives of the formula III where R⁴ and R⁵=hydrogen can then, according to the processes A and B mentioned above, be converted into the desired heteroaroyl-substituted serineamides of the formula I.

Heteroaroyl derivatives of the formula III 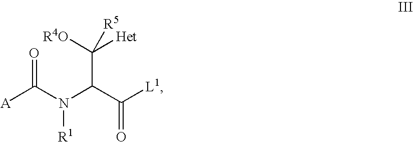
where A, Het, R¹ and R⁴ and R⁵ are as defined above and L¹ is hydroxyl or C₁-C₆-alkoxy also form part of the subject-matter of the present invention.

The particularly preferred embodiments of the intermediates with respect to the variables correspond to those of the radicals A, Het, R¹ and R⁴ and R⁵ of formula I.

Particular preference is given to heteroaroyl derivatives of the formula III in which
A is 5- or 6-membered heteroaryl selected from the group consisting of thienyl, furyl, pyrazolyl, imidazolyl, thiazolyl, oxazolyl and pyridyl;
   where the heteroaryl radicals mentioned may be partially or fully halogenated and/or may carry 1 to 3 radicals from the group consisting of C₁-C₆-alkyl, C₃-C₆-cycloalkyl and C₁-C₆-haloalkyl;
Het is mono- or bicyclic heteroaryl selected from the group consisting of thienyl, thiazolyl, tetrazolyl, pyridyl and indolyl,
   where the heteroaryls mentioned may be partially or fully halogenated and/or may carry 1 to 2 radicals from the group consisting of nitro, C₁-C₄-alkyl, C₁-C₄-haloalkyl, hydroxyl, C₁-C₄-alkoxy, C₁-C₄-haloalkoxy, hydroxy-carbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)-amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)aminocarbonylamino;
R¹ is hydrogen;
R⁴ is hydrogen, C₁-C₄-alkylcarbonyl, C₁-C₄-alkylaminocarbonyl, di-(C₁-C₄-alkyl)-aminocarbonyl, phenylaminocarbonyl, N—(C₁-C₄-alkyl)-N-(phenyl)aminocarbonyl, SO₂CH₃, SO₂CF₃ or SO₂(C₆H₅); and
R⁵ is hydrogen.

The examples below serve to illustrate the invention.

### PREPARATION EXAMPLES

### Example 1

### rac-erythro-N-(2-hydroxy-1-methylcarbamoyl-2-pyridin-3-ylethyl)-1-methyl-3-trifluoro-methyl-1H-pyrazole-4-carboxamide (Tab. 3. No. 3.3)

### 1.1) Diethyl 2-[(1-methyl-3-trifluoromethyl-1H-pyrazole-4-carbonyl)amino]malonate

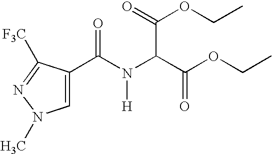

5.00 g (23.6 mmol) of diethyl aminomalonate hydrochloride were dissolved in methylene chloride, and a little THF, 4.59 g (23.6 mmol) of 1-methyl-3-trifluoromethyl-1H-pyrazole-4-carboxylic acid and 7.17 g (70.9 mmol) of triethylamine were added. With ice-cooling, 6.01 g (23.6 mmol) of bis-(2-oxo-3-oxazolidinyl)phosphoryl chloride were then added. The reaction mixture was stirred with ice-cooling for 2 h and at room temperature (RT) for 14 h. The solvents were then removed by distillation and the residue taken up in ethyl acetate, washed with 10% HCl, water and saturated NaHCO₃ solution, dried and concentrated. This gave 7.30 g (88.1%) of the title compound as a yellow powder.

¹H-NMR (DMSO): δ=1.20 (t, 6H); 3.95 (s, 3H); 4.20 (m, 4H); 5.25 (d, 1H); 8.55 (s, 1H); 9.05 (d, 1H).

### 1.2) Monoethyl rac-2-[(1-methyl-3-trifluoromethyl-1H-pyrazole-4-carbonyl)amino]-malonate

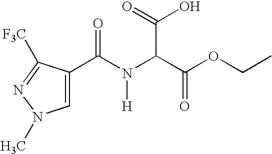

7.30 g (20.8 mmol) of diethyl 2-[(1-methyl-3-trifluoromethyl-1H-pyrazole-4-carbonyl)amino]malonate were dissolved in dioxane, and 25 ml of 1 M NaOH were added dropwise at RT. After 14 h of stirring at RT, the solution was concentrated using a rotary evaporator and extracted with diethyl ether, and the phases were separated. Ethyl acetate was added to the aqueous phase, and 14 ml of 1 M H₂SO₄ were added dropwise with ice-cooling. The organic phase was separated off, and the aqueous phase was extracted. The combined organic phases were dried and the solvent was removed. This gave 5.60 g (83.5%) of the title compound as a beige powder.

¹H-NMR (DMSO) δ=:1.20 (t, 3H); 3.95 (s, 3H); 4.20 (m, 2H); 5.20 (d, 1H); 8.55 (s, 1H); 8.95 (d, 1H).

### 1.3) Ethyl rac-erythro-3-hydroxy-2-[(1-methyl-3-trifluoromethyl-1H-pyrazole-4-carbonyl)-amino]-3-pyridin-3-ylpropionate (Tab. 2, No. 2.3)

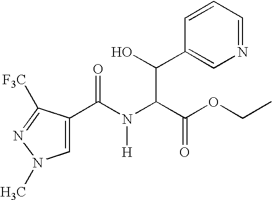

0.90 g (2.78 mmol) of monoethyl rac-2-[(1-methyl-3-trifluoromethyl-1H-pyrazole-4-carbonyl)amino]malonate was dissolved in THF, and 0.30 g (2.78 mmol) of pyridin-3-aldehyde and 0.28 g (2.78 mmol) of triethylamine were added dropwise. The mixture was stirred at RT for 14 h. The solvent was then removed by distillation, the residue was taken up in methylene chloride and the solution was washed, dried and concentrated. This gave 0.47 g (43.8%) of the title compound as a colorless powder.

¹H-NMR (DMSO): δ=1.10 (t, 3H); 3.90 (s, 3H); 4.1 (m, 2H); 4.65 (t, 1H); 4.90 (q, 1H); 6.00 (d, 1H); 7.30 (q, 1H); 7.80 (d, 1H); 8.30 (s, 1H); 8.40 (d, 1H); 8.50 (d, 1H); 8.55 (s, 1H).

### 1.4) rac-erythro-N-(2-hydroxy-1-methylcarbamoyl-2-pyridin-3-ylethyl)-1-methyl-3-trifluoromethyl-1H-pyrazole-4-carboxamide (Tab. 3, No. 3.3)

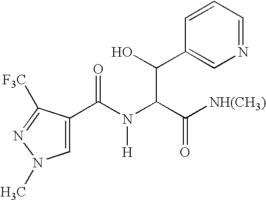

0.47 g (1.22 mmol) of ethyl rac-erythro-3-hydroxy-2-[(1-methyl-3-trifluoromethyl-1H-pyrazole-4-carbonyl)amino]-3-pyridin-3-ylpropionate was dissolved in methanol, and for a period of 4 h, methylamine was introduced into the solution at 5-10° C. After 14 h of stirring, the solvent was removed by distillation. This gave 0.41 g (90.6%) of the title compound as a solid (m.p. 175° C.).

### Example 2

rac-erythro-N-(2-hydroxy-1-methylcarbamoyl-2-quinolin-3-ylethyl)-4-trifluoromethyl-thiophene-3-carboxamide (Tab. 3. No. 3.15)

### 2.1) Diethyl 2-[(4-trifluoromethylthiophene-3-carbonyl)amino]malonate

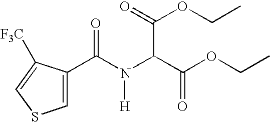

16.2 g (76.5 mmol) of diethyl aminomalonate hydrochloride were dissolved in methylene chloride, and a little THF, 15.0 g (76.5 mmol) of 3-trifluoromethyl-1H-thiophene-4-carboxylic acid and 23.2 g (229 mmol) of triethylamine were added. With ice-cooling, 19.5 g (76.5 mmol) of bis-(2-oxo-3-oxazolidinyl)phosphoryl chloride were then added. The reaction mixture was stirred with ice-cooling for 2 h and at RT for 14 h. The solvents were then removed by distillation and the residue was taken up in ethyl acetate, washed with 10% HCl, water and saturated NaHCO₃ solution, dried and concentrated. This gave 23.5 g (87.0%) of the title compound as a yellow powder.

¹H-NMR (DMSO): δ=1.20 (t, 6H); 4.20 (m, 4H); 5.30 (d, 1H); 8.25 (s, 1H) 8.30 (s, 1H); 9.40 (d, 1H).

### 2.2) Monoethyl rac-2-[(4-trifluoromethylthiophene-3-carbonyl)amino]malonate

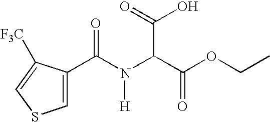

23.5 g (66.6 mmol) of diethyl 2-[(4-trifluoromethylthiophene-3-carbonyl)amino]malonate were dissolved in dioxane, and 66.6 ml of 1 M NaOH were added dropwise at RT. After 62 h of stirring at RT, the solution was concentrated using a rotary evaporator and extracted with diethyl ether, and the phases were separated. Ethyl acetate was added to the aqueous phase, and 45 ml of 1 M H₂SO₄ were added dropwise with ice-cooling. The organic phase was separated off, and the aqueous phase was extracted. The combined organic phases were dried and the solvent was removed. This gave 12.3 g (56.8%) of the title compound as a colorless viscous oil.

¹H-NMR (DMSO): δ=1.20 (t, 3H); 4.20 (m, 2H); 5.15 (d, 1H); 8.30 (s, 1H); 8.35 (s, 1H); 9.20 (d, 1H).

### 2.3) Ethyl rac-erythro-3-hydroxy-3-quinolin-3-yl-2-[(4-trifluoromethylthiophene-3-carbonyl)amino]propionate (Tab. 2, No. 2.9)

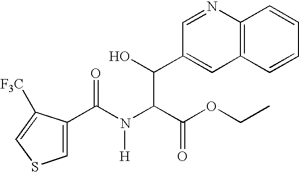

0.80 g (1.97 mmol) of monoethyl rac-2-[(4-trifluoromethylthiophene-3-carbonyl)amino]-malonate was dissolved in THF, and 0.32 g (1.97 mmol) of quinoline-3-aldehyde and 0.20 g (1.97 mmol) of triethylamine were added dropwise. This mixture was stirred at RT for 96 h. The solvent was then removed by distillation, the residue was taken up in methylene chloride and the solution was washed, dried and concentrated.

Chromatographic purification (silica gel; cyclohexane/ethyl acetate) gave 0.39 g (45.1%) of the title compound as a colorless powder.

¹H-NMR (DMSO): δ=1.15 (t, 3H); 4.1 (m, 2H); 4.75 (t, 1H); 5.20 (q, 1H); 6.20 (d, 1H); 7.60 (t, 1H); 7.70 (t, 1H); 8.0 (m, 3H); 8.20 (s, 1H); 8.35 (s, 1H); 8.90 (d, 1H); 8.95 (s, 1H).

### 2.4) rac-erythro-N-(2-hydroxy-1-methylcarbamoyl-2-quinolin-3-ylethyl)-4-trifluoromethyl-thiophene-3-carboxamide (Tab. 3, No. 3.15)

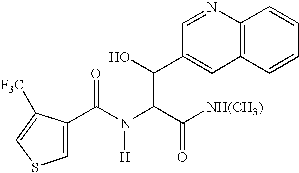

0.26 g (0.59 mmol) of ethyl rac-erythro-3-hydroxy-3-quinolin-3-yl-2-[(4-trifluoromethyl-thiophene-3-carbonyl)amino]propionate were dissolved in methanol, and for a period of 4 h, methylamine was introduced into the solution at 5-10° C. After 14 h of stirring, the solvent was removed by distillation. This gave 0.22 g (88.3%) of the title compound as a solid (m.p. 206° C.).

In addition to the above compounds, further heteroaroyl derivatives of the formula III and heteroaroyl-substituted serineamides of the formula I which were prepared or are preparable in a manner analogously to the processes described above are listed in Tables 2 and 3 below.

TABLE 2

| | |
|---|---|
| | III |
| 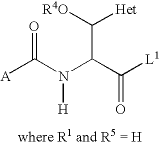 | |

| | | | | | threo: | | |
|---|---|---|---|---|---|---|---|
| No. | A | Het | R⁴ | L¹ | erythro | Config. | 1H-NMr, 400 MHz, DMSO-d6, δ [ppm] |
| 2.1 | 1-CH₃-3-CF₃-pyrazol-4-yl | pyrid-2-yl | H | OC₂H₅ | 0:1 | rac. | 1.1 (t, 3H); 3.9 (s, 3H); 4.0 (m, 2H); 5.00 (m, |
| | | | | | | | 2H); 5.95 (d, 1H); 7.25 (q, 1H); 7.45 (d, 1H); |
| | | | | | | | 7.80 (t, 1H); 8.10 (s, 1H); 8.25 (s, 1H); 8.50 (d, |
| | | | | | | | 1H); 8.70 (d, 1H) |
| 2.2 | 1-CH₃-3-CF₃-pyrazol-4-yl | 3-CH₃-pyrid-2-yl | H | OC₂H₅ | 0:1 | rac. | 1.0 (t, 3H); 3.9 (s, 3H); 4.1 (m, 2H); 5.0 (m, |
| | | | | | | | 2H); 5.90 (d, 1H); 7.10 (d, 1H); 7.25 (d, 1H); |
| | | | | | | | 7.65 (t, 1H); 8.30 (d, 1H); 8.45 (s, 1H) |
| 2.3 | 1-CH₃-3-CF₃-pyrazol-4-yl | pyrid-3-yl | H | OC₂H₅ | 0:1 | rac. | 1.10 (t, 3H); 3.90 (s, 3H); 4.1 (m, 2H); 4.65 (t, |
| | | | | | | | 1H); 4.90 (q, 1H); 6.00 (d, 1H); 7.30 (q, 1H); |
| | | | | | | | 7.80 (d, 1H); 8.30 (s, 1H); 8.40 (d, 1H); 8.50 (d, |
| | | | | | | | 1H); 8.55 (s, 1H) |
| 2.4 | 1-CH₃-3-CF₃-pyrazol-4-yl | pyrid-4-yl | H | OC₂H₅ | 0:1 | rac. | 1.1 (t, 3H); 3.90 (s, 3H); 4.1 (m, 2H); 4.60 (t, |
| | | | | | | | 1H); 4.9 (q, 1H); 6.10 (d, 1H); 7.40 (d, 2H); 8.35 |
| | | | | | | | (s, 1H); 8.50 (d, 2H); 8.50 (d, 1H) |
| 2.5 | 4-CF₃-thien-3-yl | pyrid-3-yl | H | OC₂H₅ | 0:1 | rac. | 1.15 (t, 3H); 4.15 (m, 2H); 4.60 (m, 1H); 4.95 |
| | | | | | | | (m, 1H); 6.15 (d, 1H); 7.45 (m, 2H); 8.00 (s, |
| | | | | | | | 1H); 8.25 (s, 1H); 8.50 (m, 2H); 8.90 (d, 1H) |
| 2.6 | 4-CF₃-thien-3-yl | 6-CF₃-pyrid-3-yl | H | OC₂H₅ | 1:4 | rac. | 1.15 (t, 3H); 4.10 (m, 2H); 4.80 (t, 1H); 5.25 (t, |
| | | | | | | | 1H); 6.30 (d, 1H); 7.70 (d, 1H); 7.95 (s, 1H); |
| | | | | | | | 8.20 (s, 1H); 8.70 (d, 1H); 8.80 (d, 1H); 8.90 (s, |
| | | | | | | | 1H) |
| 2.7 | 4-CF₃-thien-3-yl | 5-(4-F-phenyl)- | H | OC₂H₅ | 0:1 | rac. | 1.15 (t, 3H); 4.15 (m, 2H); 4.75 (t, 1H); 5.05 (q, |
| | | pyrid-3-yl | | | | | 1H); 6.10 (d, 1H); 7.30 (t, 2H); 7.70 (q, 2H); |
| | | | | | | | 7.90 (s, 1H); 8.05 (s, 1H); 8.20 (s, 1H); 8.55 (s, |
| | | | | | | | 1H); 8.75 (s, 1H); 8.90 (d, 1H) |
| 2.8 | 4-CF₃-thien-3-yl | 4-Cl-pyrid-3-yl | H | OC₂H₅ | 1:4 | rac. | 1.15 (t, 3H); 4.10 (m, 2H); 4.60 (t, 1H); 4.95 |
| | | | | | | | (q, 1H); 6.15 (d, 1H); 7.45 (d, 1H); 7.85 (d, 1H); |
| | | | | | | | 7.95 (s, 1H); 8.20 (s, 1H); 8.40 (s, 1H); 8.85 (d, |
| | | | | | | | 1H) |
| 2.9 | 4-CF₃-thien-3-yl | quinolin-3-yl | H | OC₂H₅ | 0:1 | rac. | 1.15 (t, 3H); 4.15 (m, 2H); 4.80 (t, 1H); 5.20 (q, |
| | | | | | | | 1H); 6.20 (d, 1H); 7.60 (t, 1H); 7.75 (t, 1H); 8.0 |
| | | | | | | | (m, 3H); 8.30 (s, 1H); 8.85 (s, 1H); 8.90 (d, 1H); |
| | | | | | | | 8.95 (s, 1H) |

TABLE 3

| | |
|---|---|
| | I |
| 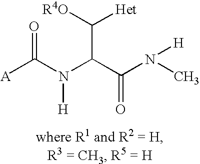 | |

| | | | | threo: | | m.p. [° C.] or |
|---|---|---|---|---|---|---|
| No. | A | Het | R⁴ | erythro | Config. | ¹H-NMr, 400 MHz, d4-MeOH, δ [ppm] |
| 3.1 | 1-CH₃-3-CF₃-pyrazol-4-yl | pyrid-2-yl | H | 0:1 | rac. | 184° C. |
| 3.2 | 1-CH₃-3-CF₃-pyrazol-4-yl | 6-CH₃-pyrid-2-yl | H | 0:1 | rac. | 213° C. |
| 3.3 | 1-CH₃-3-CF₃-pyrazol-4-yl | pyrid-3-yl | H | 0:1 | rac. | 175° C. |
| 3.4 | 1-CH₃-3-CF₃-pyrazol-4-yl | pyrid-4-yl | H | 0:1 | rac. | 220° C. |
| 3.5 | 1-CH₃-3-CF₃-pyrazol-4-yl | 2-thienyl | H | 3:2 | rac. | 207° C. |
| 3.6 | 1-CH₃-3-CF₃-pyrazol-4-yl | 2-thienyl | C(O)N(CH₃)₂ | 1:1 | rac. | 195° C. |
| 3.7 | 1-CH₃-3-CF₃-pyrazol-4-yl | 3-thienyl | H | 0:1 | rac. | 162° C. |
| 3.8 | 1-CH₃-3-CF₃-pyrazol-4-yl | 3-furanyl | H | 0:1 | rac. | 2.40 (d, 3H); 3.95 (s, 3H); 4.70 (d, 1H); |
| | | | | | | 5.95 (d, 1H); 6.45 (d, 1H); 7.4 (s, 1H); 7.5 |
| | | | | | | (s, 1H); 8.1 (s, 1H); 8.2 (s, 2H) |
| 3.9 | 4-CF₃-thien-3-yl | pyrid-2-yl | H | 0:1 | rac. | 152° C. |
| 3.10 | 4-CF₃-thien-3-yl | pyrid-3-yl | H | 0:1 | rac. | 155° C. |
| 3.11 | 4-CF₃-thien-3-yl | 4-Cl-pyrid-3-yl | H | 1:4 | rac. | 225° C. |
| 3.12 | 4-CF₃-thien-3-yl | 6-CF₃-pyrid-3-yl | H | 1:4 | rac. | 185° C. |
| 3.13 | 4-CF₃-thien-3-yl | 5-(4-F-phenyl)-pyrid-3-yl | H | 0:1 | rac. | 223° C. |
| 3.14 | 4-CF₃-thien-3-yl | pyrid-4-yl | H | 0:1 | rac. | 234° C. |
| 3.15 | 4-CF₃-thien-3-yl | quinolin-3-yl | H | 0:1 | rac. | 206° C. |
| 3.16 | 1-CH₃-3-CF₃-4-pyrazolyl | 2-pyridyl | C(O)N(CH₃)₂ | 0:1 | rac | m/z 442 |
| 3.17 | 1-CH₃-3-CF₃-4-pyrazolyl | 5-pyrazolyl | H | 1:1 | rac | 207° C. |
| 3.18 | 1-CH₃-3-CF₃-4-pyrazolyl | 5-pyrazolyl | C(O)CH₃ | 0:1 | rac | m/z 414 |
| 3.19 | 1-CH₃-3-CF₃-4-pyrazolyl | 5-pyrazolyl | C(O)CH₃ | 1:1 | rac | 202° C. |
| 3.20 | 1-CH₃-3-CF₃-4-pyrazolyl | 5-pyrazolyl | C(O)N(CH₃)₂ | 1:1 | rac | m/z 443 |
| 3.21 | 1-CH₃-3-CF₃-4-pyrazolyl | 2-furyl | H | 2:3 | rac | m/z 360 |
| 3.22 | 1-CH₃-3-CF₃-4-pyrazolyl | 2-furyl | C(O)CH₃ | 2:3 | rac | m/z 402 |
| 3.23 | 1-CH₃-3-CF₃-4-pyrazolyl | 2-furyl | C(O)N(CH₃)₂ | 1:3 | rac | m/z 431 |
| 3.24 | 1-CH₃-3-CF₃-4-pyrazolyl | 5-Br-2-furyl | H | 1:2 | rac | 190° C. |
| 3.25 | 1-CH₃-3-CF₃-4-pyrazolyl | 5-Br-2-furyl | C(O)CH₃ | 1:2 | rac | 180° C. |
| 3.26 | 1-CH₃-3-CF₃-4-pyrazolyl | 5-(CH₂OH)-2-furyl | H | 1:1 | rac | m/z 390 |
| 3.27 | 1-CH₃-3-CF₃-4-pyrazolyl | 5-(CH₂OH)-2-furyl | H | 2:1 | rac | m/z 390 |
| 3.28 | 1-CH₃-3-CF₃-4-pyrazolyl | 5-[CH₂—O(CO)CH₃]-2-furyl | C(O)CH₃ | 1:0 | rac | m/z 474 |
| 3.29 | 1-CH₃-3-CF₃-4-pyrazolyl | 5-[CH₂—O(CO)CH₃]-2-furyl | C(O)CH₃ | 1:1 | rac | m/z 474 |
| 3.30 | 1-CH₃-3-CF₃-4-pyrazolyl | 5-[CH₂—O(CO)CH₃]-2-furyl | C(O)CH₃ | 1:3 | rac | m/z 474 |
| 3.31 | 1-CH₃-3-CF₃-4-pyrazolyl | 5-[CH₂—O(CO)-N(CH₃)₂]-2-furyl | C(O)N(CH₃)₂ | 2:1 | rac | m/z 532 |
| 3.32 | 1-CH₃-3-CF₃-4-pyrazolyl | 5-[CH₂—O(CO)-N(CH₃)₂]-2-furyl | C(O)N(CH₃)₂ | 1:1 | rac | m/z 532 |
| 3.33 | 1-CH₃-3-CF₃-4-pyrazolyl | 1-CH₃-3-pyrazolyl | H | 1:0 | rac | 220° C. |
| 3.34 | 1-CH₃-3-CF₃-4-pyrazolyl | 1-CH₃-3-pyrazolyl | H | 1:4 | rac | 170° C. |
| 3.35 | 1-CH₃-3-CF₃-4-pyrazolyl | 1-CH₃-3-pyrazolyl | C(O)N(CH₃)₂ | 1:0 | rac | m/z 445 |
| 3.36 | 1-CH₃-3-CF₃-4-pyrazolyl | 1-CH₃-4-Cl-pyrazolyl | H | 1:5 | rac | m/z 409 |
| 3.37 | 1-CH₃-3-CF₃-4-pyrazolyl | 1-CH₃-4-Cl-pyrazolyl | H | 3:1 | rac | m/z 409 |
| 3.38 | 1-CH₃-3-CF₃-4-pyrazolyl | 1-CH₃-4-Cl-pyrazolyl | C(O)CH₃ | 1:5 | rac | m/z 451 |
| 3.39 | 1-CH₃-3-CF₃-4-pyrazolyl | 1-CH₃-4-Cl-pyrazolyl | C(O)CH₃ | 3:1 | rac | m/z 451 |
| 3.40 | 1-CH₃-3-CF₃-4-pyrazolyl | 1-CH₃-4-Cl-pyrazolyl | C(O)CH₃ | 0:1 | rac | 215° C. |
| 3.41 | 1-CH₃-3-CF₃-4-pyrazolyl | 1-CH₃-4-Cl-pyrazolyl | C(O)N(CH₃)₂ | 0:1 | rac | m/z 480 |
| 3.42 | 1-CH₃-3-CF₃-4-pyrazolyl | 1-CH₃-4-Cl-pyrazolyl | C(O)N(CH₃)₂ | 3:1 | rac | m/z 480 |
| 3.43 | 1-CH₃-3-CF₃-4-pyrazolyl | 4-imidazolium-hydrochlorid | H | 1:0 | rac | 205° C. |
| 3.44 | 1-CH₃-3-CF₃-4-pyrazolyl | 4-imidazolium-hydrochlorid | H | 1:1 | rac | 80° C. |
| 3.45 | 1-CH₃-3-CF₃-4-pyrazolyl | 4-CH₃-5-1,2,4-thiazolyl | H | 1:0 | rac | 211° C. |
| 3.46 | 1-CH₃-3-CF₃-4-pyrazolyl | 4-CH₃-5-1,2,4-thiazoiyl | H | 0:1 | rac | 248° C. |
| 3.47 | 1-CH₃-3-CF₃-4-pyrazolyl | 4-CH₃-5-1,2,4-thiazolyl | C(O)CH₃ | 0:1 | rac | 242° C. |
| 3.48 | 1-CH₃-3-CF₃-4-pyrazolyl | 4-CH₃-5-1,2,4-thiazolyl | C(O)OCH₃ | 0:1 | rac | 204° C. |
| 3.49 | 1-CH₃-3-CF₃-4-pyrazolyl | 4-CH₃-5-1,2.4-thiazolyl | C(O)N(CH₃)₂ | 1:0 | rac | 209° C. |
| 3.50 | 1-CH₃-3-CF₃-4-pyrazolyl | 4-CH₃-5-1,2,4-thiazolyl | C(O)N(CH₃)₂ | 0:1 | rac | 236° C. |
| 3.51 | 1-CH₃-3-CF₃-4-pyrazolyl | 2-thiazolyl | H | 1:7 | rac | m/z 377 |
| 3.52 | 1-CH₃-3-CF₃-4-pyrazolyl | 2-thiazolyl | H | 3:1 | rac | m/z 377 |
| 3.53 | 1-CH₃-3-CF₃-4-pyrazolyl | 2-thiazolyl | C(O)CH₃ | 1:0 | rac | m/z 419 |
| 3.54 | 1-CH₃-3-CF₃-4-pyrazolyl | 2-thiazolyl | C(O)CH₃ | 1:3 | rac | m/z 419 |
| 3.55 | 1-CH₃-3-CF₃-4-pyrazolyl | 2-thiazolyl | C(O)CH₃ | 0:1 | rac | m/z 419 |
| 3.56 | 1-CH₃-3-CF₃-4-pyrazolyl | 2-thiazolyl | C(O)N(CH₃)₂ | 1:0 | rac | m/z 448 |
| 3.57 | 1-CH₃-3-CF₃-4-pyrazolyl | 2-thiazolyl | C(O)N(CH₃)₂ | 2:1 | rac | m/z 448 |
| 3.58 | 1-CH₃-3-CF₃-4-pyrazolyl | 2-thiazolyl | C(O)N(CH₃)₂ | 2:3 | rac | m/z 448 |
| 3.59 | 1-CH₃-3-CF₃-4-pyrazolyl | 2-thiazolyl | C(O)N(CH₃)₂ | 1:4 | rac | m/z 448 |
| 3.60 | 1-CH₃-3-CF₃-4-pyrazolyl | 3-CH₃-2-thiazolyl | H | 1:0 | rac | m/z 405 |
| 3.61 | 1-CH₃-3-CF₃-4-pyrazolyl | 2-benzofuranyl | H | 0:1 | rac | m/z 410 |
| 3.62 | 1-CH₃-3-CF₃-4-pyrazolyl | 2-benzofuranyl | H | 1:1 | rac | m/z 410 |
| 3.63 | 1-CH₃-3-CF₃-4-pyrazolyl | 1-CH₃-2-benzimidazolyl | H | 1:0 | rac | 230° C. |
| 3.64 | 1-CH₃-3-CF₃-4-pyrazolyl | 1-CH₃-2-benzimidazolyl | C(O)CH₃ | 1:1 | rac | m/z 466 |
| 3.65 | 1-CH₃-3-CF₃-4-pyrazolyl | 1-CH₃-2-benzimidazolyl | C(O)N(CH₃)₂ | 1:1 | rac | m/z 495 |
| 3.66 | 4-CF₃-3-thienyl | 2-pyridyl | C(O)CH₃ | 0:1 | rac | m/z 415.39 |
| 3.67 | 4-CF₃-3-thienyl | 3-pyridyl | C(O)CH₃ | 0:1 | rac | m/z 415.39 |
| 3.68 | 4-CF₃-3-thienyl | 4-pyridyl | C(O)CH₃ | 1:2 | rac | 150° C. |
| 3.69 | 4-CF₃-3-thienyl | 2-Cl-5-pyridyl | C(O)N(CH₃)₂ | 0:1 | rac | 200° C. |
| 3.70 | 4-CF₃-3-thienyl | 2-Cl-5-pyridyl | C(O)N(CH₃)₂ | 1:1 | rac | 186° C. |

Biological Activity

The heteroaroyl-substituted serineamides of the formula I and their agriculturally useful salts are suitable, both in the form of isomer mixtures and in the form of the pure isomers, as herbicides. The herbicidal compositions comprising compounds of the formula I control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds of the formula I, or herbicidal compositions comprising them, can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, lpomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds of the formula I may also be used in crops which tolerate the action of herbicides owing to breeding, including genetic engineering methods.

The compounds of the formula I, or the compositions comprising them, can be used for example in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading or watering. The use forms depend on the intended purpose; in any case, they should guarantee the finest possible distribution of the active ingredients according to the invention.

The herbicidal compositions comprise a herbicidally effective amount of at least one compound of the formula I or an agriculturally useful salt of 1, and auxiliaries which are customary for the formulation of crop protection agents.

Suitable as inert auxiliaries are essentially the following:

mineral oil fractions of medium to high boiling point, such as kerosene and diesel oil, furthermore coal tar oils and oils of vegetable or animal origin, aliphatic, cyclic and aromatic hydrocarbons, e.g. paraffins, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, e.g. amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the substrates, either as such or dissolved in an oil or solvent, can be homogenized in water by means of a wetting agent, tackifier, dispersant or emulsifier. Alternatively, it is also possible to prepare concentrates comprising active substance, wetting agent, tackifier, dispersant or emulsifier and, if desired, solvent or oil, which are suitable for dilution with water.

Suitable surfactants (adjuvants) are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active substances together with a solid carrier.

Granules, e.g. coated granules, impregnated granules and homogeneous granules, can be prepared by binding the active ingredients to solid carriers. Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

The concentrations of the compounds of the formula I in the ready-to-use preparations can be varied within wide ranges. In general, the formulations comprise approximately from 0.001 to 98% by weight, preferably 0.01 to 95% by weight of at least one active ingredient. The active ingredients are employed in a purity of from 90% to 100%, preferably 95% to 100% (according to NMR spectrum).

The formulation examples below illustrate the preparation of such compositions:
I. 20 parts by weight of an active ingredient of the formula I are dissolved in a mixture composed of 80 parts by weight of alkylated benzene, 10 parts by weight of the adduct of from 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active ingredient of the formula I.
II. 20 parts by weight of an active ingredient of the formula I are dissolved in a mixture composed of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active ingredient of the formula I.
III. 20 parts by weight of an active ingredient of the formula I are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active ingredient of the formula I.
IV. 20 parts by weight of an active ingredient of the formula I are mixed thoroughly with 3 parts by weight of sodium diisobutylnaphthalenesulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20 000 parts by weight of water gives a spray mixture which comprises 0.1% by weight of the active ingredient of the formula I.
V. 3 parts by weight of an active ingredient of the formula I are mixed with 97 parts by weight of finely divided kaolin. This gives a dust which comprises 3% by weight of the active ingredient of the formula I.
VI. 20 parts by weight of an active ingredient of the formula I are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.
VII. 1 part by weight of an active ingredient compound of the formula I is dissolved in a mixture composed of 70 parts by weight of cyclohexanone, 20 parts by weight of ethoxylated isooctylphenol and 10 parts by weight of ethoxylated castor oil. This gives a stable emulsion concentrate.
VIII. 1 part by weight of an active ingredient of the formula I is dissolved in a mixture composed of 80 parts by weight of cyclohexanone and 20 parts by weight of Wettol® 31 (=nonionic emulsifier based on ethoxylated castor oil). This gives a stable emulsion concentrate.

The compounds of the formula I or the herbicidal compositions can be applied pre- or post-emergence. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

The rates of application of the compound of the formula I are from 0.001 to 3.0, preferably 0.01 to 1.0, kg/ha of active substance (a.s.), depending on the control target, the season, the target plants and the growth stage.

To widen the spectrum of action and to achieve synergistic effects, the heteroaroyl-substituted serineamides of the formula I may be mixed with a large number of representatives of other herbicidal or growth-regulating active ingredient groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-(het)aroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydro-benzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, 2-phenylpropionic acid and its derivatives, pyrazoles, phenylpyrazoles, pyridazines, pyrmidinecarboxylic acid and its derivatives, pyrimidyl ethers, sulfonamides, sulfonylureas, triazines, triazinones, triazolinones, triazolecarboxamides and uracils.

It may furthermore be beneficial to apply the compounds of the formula I alone or in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

### Use Examples

The herbicidal activity of the heteroaroyl-substituted serineamides of the formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover causes uniform germination of the test plants, unless this has been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment. The rate of application for the post-emergence treatment was 1.0 or 0.5 kg/ha of a.s. (active substance).

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100.100 means no emergence of the plants, or complete destruction of at least the aerial parts, and 0 means no damage, or normal course of growth.

The plants used in the greenhouse experiments belonged to the following species:

| Scientific name | Common name |
|---|---|
| Amaranthus retroflexus | pig weed |
| Chenopodium album | lambsquarters |
| Setaria viridis | green foxtail |

At application rates of 1.0 kg/ha, the compound 3.1 (Table 3) showed very good post-emergence action against the unwanted plants pig weed, lambsquarters and green foxtail.

Furthermore, compound 3.2 (Table 3), applied by the post-emergence method, effected, at application rates of 0.5 kg/ha, very good control of the harmful plants pig weed, lambsquarters and green foxtail.

The activity of compound 3.5 (Table 3), applied by the post-emergence method, at application rates of 1.00 kg/ha, against the unwanted plants pig weed, lambsquarters and green foxtail was very good.

Compound 3.6 (Table 3), at application rates of 0.5 kg/ha, effected very good post-emergence control of the harmful plants pig weed, lambsquarters and green foxtail.

Compound 3.19 and 3.54 (Table 3), at application rates of 0.5 kg/ha, effected very good post-emergence control of the harmful plants pig weed, lambsquarters and green foxtail.

Furthermore, compounds 3.17, 3.20, 3.33, 3.36, 3.41, 3.42, 3.51, 3.52, 3.57 and 3.59 (Table 3), applied by the post-emergence method, effected, at application rates of 1.0 kg/ha, very good control of the harmful plants pig weed, lambsquarters and green foxtail.

1. A compound which is a heteroaroyl-substituted serineamide of the formula I 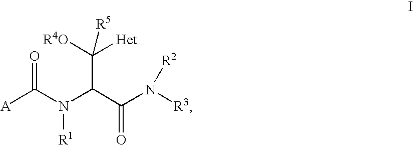
in which the variables are as defined below:
A is 5- or 6-membered heteroaryl having one to four nitrogen atoms or one to three nitrogen atoms and one oxygen or sulfur atom or one oxygen or sulfur atom, which heteroaryl may be partially or fully halogenated and/or may carry 1 to 3 radicals selected from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
Het is mono- or bicyclic heteroaryl having 5 to 10 ring members comprising 1 to 4 heteroatoms selected from the group consisting of nitrogen, oxygen and sulfur, which heteroaryl may be partially or fully halogenated and/or may carry 1 to 3 radicals selected from the group consisting of cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di-(C₁-C₆-alkyl)amino, C₁-C₆-alkylsulfonylamino, C₁-C₆-haloalkylsulfonylamino, aminocarbonylamino, (C₁-C₆-alkylamino)carbonylamino, di-(C₁-C₆-alkyl)aminocarbonylamino, aryl and aryl-(C₁-C₆-alkyl);
R¹, R² are independently hydrogen, hydroxyl or C₁-C₆-alkoxy;
R³ is C₁-C₆-alkyl, C₁-C₄-cyanoalkyl or C₁-C₆-haloalkyl;
R⁴ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di-(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, di-(C₁-C₆-alkyl)aminothiocarbonyl, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-(di-C₁-C₆-alkylamino)imino-C₁-C₆-alkyl or tri-C₁-C₄-alkylsilyl,
where the alkyl, cycloalkyl and alkoxy radicals mentioned may be partially or fully halogenated and/or may carry one to three of the following groups: cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di-(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
phenyl, phenyl-C₁-C₆-alkyl, phenylcarbonyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl, heterocyclyl, heterocyclyl-C₁-C₆-alkyl, heterocyclylcarbonyl, heterocyclylcarbonyl-C₁-C₆-alkyl, heterocyclyloxycarbonyl, heterocyclylaminocarbonyl, heterocyclylsulfonylaminocarbonyl, N—(C₁-C₆-alkyl)-N-(heterocyclyl)aminocarbonyl, or heterocyclyl-C₁-C₆-alkylcarbonyl,
where the phenyl and the heterocyclyl radical of the 17 last-mentioned substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R⁶;
R⁵ is hydrogen or C₁-C₆-alkyl;
R⁶ is C₁-C₆-alkyl, C₁-C₆-haloalkyl or phenyl,
where the phenyl radical may be partially or fully halogenated and/or may carry one to three of the following groups: C₁-C₆-alkyl, C₁-C₆-haloalkyl or C₁-C₆-alkoxy;
or an agriculturally useful salt thereof.

2. The compound according to claim 1 where A is 5- or 6-membered heteroaryl selected from the group consisting of pyrrolyl, thienyl, furyl, pyrazolyl, imidazolyl, thiazolyl, oxazolyl, tetrazolyl, pyridyl and pyrimidinyl; where the heteroaryl radicals mentioned may be partially or fully halogenated and/or may carry 1 to 3 radicals selected from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl.

3. The compound according to claim 1, where Het is mono- or bicyclic heteroaryl selected from the group consisting of furyl, thienyl, pyrazolyl, imidazolyl, thiazolyl, triazolyl, tetrazolyl, pyridyl, pyrimidinyl, quinolinyl and indolyl,
where the heteroaryl radicals mentioned may be partially or fully halogenated and/or may carry 1 to 3 radicals selected from the group consisting of nitro, C₁-C₄-alkyl, C₁-C₄-haloalkyl, hydroxyl, C₁-C₄-alkoxy, C₁-C₄-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di-(C₁-C₆-alkyl)amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)aminocarbonylamino.

4. The compound according to claim 1 where R¹, R² and R⁵ are hydrogen.

5. A process for preparing a heteroaroyl-substituted serineamide[s] of the formula I according to claim 1, wherein a serine derivative[s] of the formula V 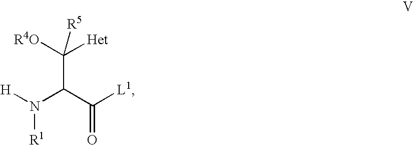
where Het and R¹, R⁴ and R⁵ are as defined in claim 1 and L¹ is a nucleophilically displaceable leaving group
is reacted with a heteroaryl acid (derivative[s]) of the formula IV 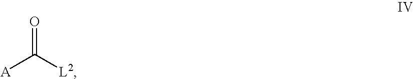
where A is as defined claim 1 and L² is a nucleophilically displaceable leaving group to give the corresponding heteroaroyl derivative[s] of the formula IV 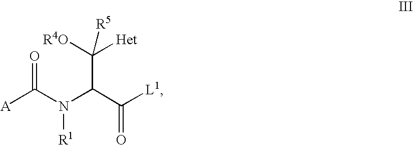
where A, Het, R¹, R⁴ and R⁵ are as defined in claim 1 and L¹ is a nucleophilically displaceable leaving group,
and the resulting heteroaroyl derivative[s] of the formula III &F is then reacted with an amine of the formula II
HNR²R³ II,
where R² and R³ are as defined in claim 1 to produce a heteroaroyl-substituted serineamide of formula 1.

6. A process for preparing a heteroaroyl-substituted serineamide[s] of the formula I according to claim 5 where R⁴ and R⁵ are hydrogen, wherein a heteroaroyl derivative[s] of the formula III where R⁴ and R⁵ are hydrogen is prepared by acylating a keto compound[s] of the formula XIII 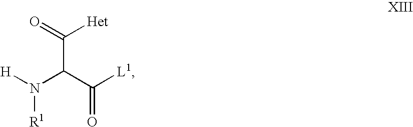
where R¹ is as defined in claim 5 and L¹ is a nucleophilically displaceable leaving group
with a heteroaryl acid (derivatives) of the formula IV to give a N-acyl keto compound[s] of the formula XII 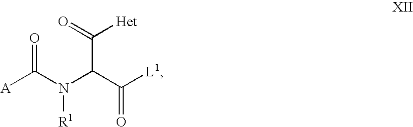
where A, Het and R¹ are as defined in claim 5 and L¹ is a nucleophilically displaceable leaving group
and subsequently reducing of the keto group to produce the heteroaroyl derivative of formula III.

7. A heteroaroyl derivative of the formula III 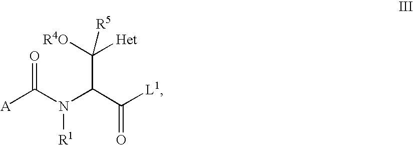
wherein
A is 5- or 6-membered heteroaryl having one to four nitrogen atoms or one to three nitrogen atoms and one oxygen or sulfur atom or one oxygen or sulfur atom, which heteroaryl may be partially or fully halogenated and/or may carry 1 to 3 radicals selected from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
Het is mono- or bicyclic heteroaryl having 5 to 10 ring members comprising 1 to 4 heteroatoms selected from the group consisting of nitrogen, oxygen and sulfur, which heteroaryl may be partially or fully halogenated and/or may carry 1 to 3 radicals selected from the group consisting of cyan, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di-(C₁-C₆-alkyl)amino, C₁-C₆-alkylsulfonylamino, C₁-C₆-haloalkylsulfonylamino, aminocarbonylamino. (C₁-C₆-alkylamino)carbonylamino, di-(C₁-C₆-alkyl)aminocarbonylamino, aryl and aryl-(C₁-C₆-alkyl);
R¹ is hydrogen, C₁-C₄-alkyl or C₁-C₄-haloalkyl;
R⁴ is hydrogen, C₁-C₆-alkyl, C₃-C₆-cycloalkyl, C₃-C₆-alkenyl, C₃-C₆-alkynyl, C₃-C₆-haloalkenyl, C₁-C₆-haloalkynyl, formyl, C₁-C₆-alkylcarbonyl, C₃-C₆-cycloalkylcarbonyl, C₂-C₆-alkenylcarbonyl, C₁-C₆-alkynylcarbonyl, C₁-C₆-alkoxycarbonyl, C₃-C₆-alkenyloxycarbonyl, C₃-C₆-alkynyloxycarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di-(C₁-C₆-alkyl)aminocarbonyl, N—(C₁-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl. N—(C₃-C₆-alkynyl)-N—(C₁-C₆-alkoxy)aminocarbonyl, di-(C₁-C₆-alkyl)aminothiocarbonyl, C₁-C₆-alkylcarbonyl-C₁-C₆-alkyl, C₁-C₆-alkoxyimino-C₁-C₆-alkyl. N—(C₁-C₆-alkylamino)imino-C₁-C₆-alkyl, N-(di-C₁-C₆-alkylamino)imino-C₁-C₆-alkyl or tri-C₁-C₄-alkylsilyl,
where the alkyl, cycloalkyl and alkoxy radicals mentioned may be partially or fully halogenated and/or may carry one to three of the following groups:
cyano, hydroxyl, C₃-C₆-cycloalkyl, C₁-C₆-alkoxy-C₁-C₄-alkyl, C₁-C₄-alkoxy-C₁-C₄-alkoxy-C₁-C₄ alkyl, C₁-C₄-alkoxy, C₁-C₄-alkylthio, di-(C₁-C₄-alkyl)amino, C₁-C₄-alkyl-C₁-C₆-alkoxycarbonylamino, C₁-C₄-alkylcarbonyl,
hydroxycarbonyl, C₁-C₄-alkoxycarbonyl, aminocarbonyl, C₁-C₄ alkylaminocarbonyl, di-(C₁-C₄-alkyl)aminocarbonyl or C₁-C₄-alkylcarbonyloxy;
phenyl, phenyl-C₁-C₆-alkyl, phenylcarbonyl, phenylcarbonyl-C₁-C₆-alkyl, phenoxycarbonyl. Phenylaminocarbonyl. Phenylsulfonylaminocarbonyl. N—(C₁-C₆-alkyl)-N-(phenyl)aminocarbonyl, phenyl-C₁-C₆-alkylcarbonyl, heterocyclyl, heterocyclyl-C₁-C₆-alkyl, heterocyclylcarbonyl, heterocyclylcarbonyl-C₁-C₆-alkyl, heterocyclyloxycarbonyl, heterocyclylaminocarbonyl,
heterocyclylsulfonylaminocarbonyl. N—(C₁-C₆-alkyl)-N-(heterocyclyl)-aminocarbonyl, or heterocyclyl-C₁-C₆-alkylcarbonyl,
where the phenyl and the heterocyclyl radical of the 17 last-mentioned substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R⁶;
R⁵ is hydrogen or C₁-C₆-alkyl;
and L¹ is a nucleophilically displaceable leaving group.

8. A composition, comprising a herbicidally effective amount of at least one heteroaroyl-substituted serineamide of the formula I or an agriculturally useful salt thereof according to claim 1 and auxiliaries customary for formulating crop protection agents.

9. A process for preparing a herbicidal composition comprising mixing a herbicidally effective amount of at least one heteroaroyl-substituted serineamide of the formula I or an agriculturally useful salt thereof according to claim 1 and auxiliaries customary for formulating crop protection agents are mixed.

10. A method for controlling unwanted vegetation, wherein a herbicidally effective amount of at least one heteroaroyl-substituted serineamide of the formula I or an agriculturally useful salt thereof according to claim 1 is allowed to act on plants, their habitat and/or on seed.

11. (canceled)

12. The compound of claim 1, wherein
A is 5- or 6-membered heteroaryl which is attached via carbon and selected from the group consisting of A1 to A14 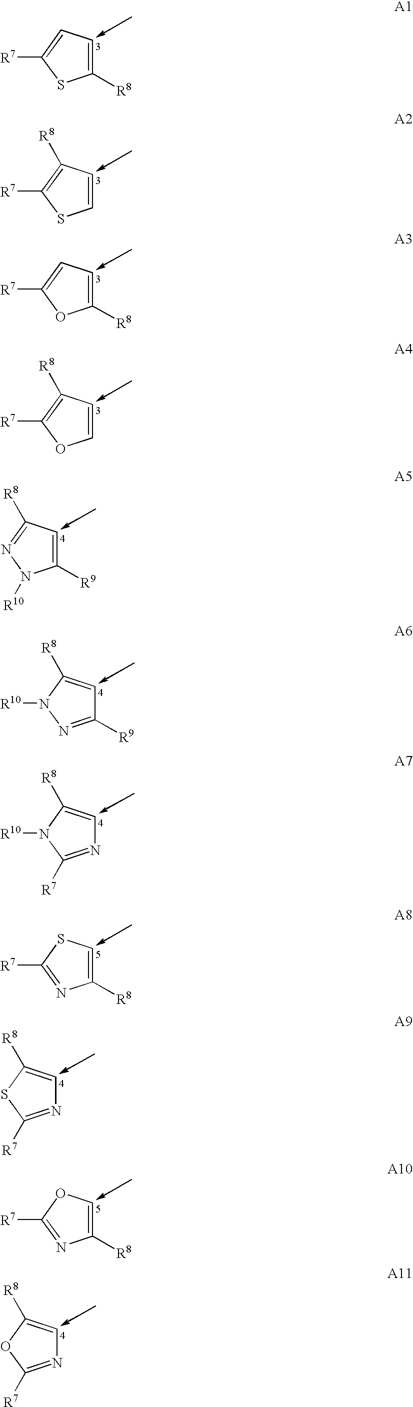 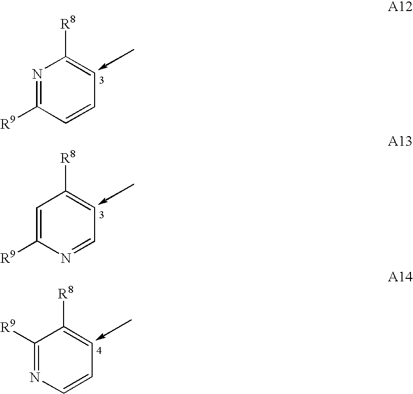
where the arrow indicates the point of attachment and
R⁷ is hydrogen, halogen, C₁-C₆-alkyl or C₁-C₆-haloalkyl;
R⁸ is halogen, C₁-C₆-alkyl, C₁-C₆-haloalkyl or C₁-C₆-haloalkoxy;
R⁹ is hydrogen, halogen, C₁-C₆-alkyl or C₁-C₆-haloalkyl;
R¹⁰ is hydrogen, C₁-C₆-alkyl, C₃-C₆-cycloalkyl, C₁-C₆-haloalkyl or C₁-C₆-alkoxy-C₁-C₄-alkyl;

13. The compound of claim 3, wherein Het is selected from the group consisting of thienyl, thiazolyl, tetrazolyl, pyridyl and indolyl,
where the heteroaryls mentioned may be partially or fully halogenated and/or may carry 1 to 2 radicals selected from the group consisting of nitro, C₁-C₄-alkyl, C₁-C₄-haloalkyl, hydroxyl, C₁-C₄-alkoxy, C₁-C₄-haloalkoxy, hydroxycarbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)aminocarbonylamino.

14. The compound of claim 3, wherein Het is Het-1, Het-2, Het-3, Het-4, Het-5 or Het-6; 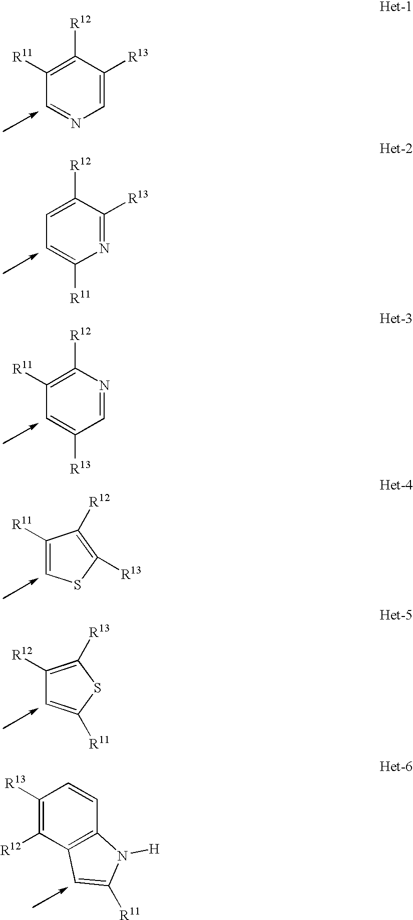
where the arrow indicates the point of attachment and
R¹¹ is hydrogen, halogen, C₁-C₆-alkyl or C₁-C₆-haloalkyl;
R¹² is hydrogen, halogen, C₁-C₆-alkyl or C₁-C₆-haloalkyl; and
R¹³ is hydrogen, halogen or C₁-C₄-alkyl;

15. The compound of claim 1, wherein
R¹ is hydrogen;
R² is hydrogen or hydroxyl; and
R³ is C₁-C₆-alkyl or C₁-C₆-haloalkyl.

16. The compound of claim 15, wherein R⁴ is
R⁴ is hydrogen, C₁-C₆-alkyl, C₃-C₆-alkenyl, C₃-C₆-alkynyl, C₁-C₆-alkylcarbonyl, C₂-C₆-alkenylcarbonyl, C₃-C₆-cycloalkylcarbonyl, C₁-C₆-alkoxycarbonyl, C₁-C₆-alkylaminocarbonyl, C₁-C₆-alkylsulfonylaminocarbonyl, di-(C₁-C₆-alkyl)aminocarbonyl, N—(C₁-C₆-alkoxy)-N—(C₁-C₆-alkyl)aminocarbonyl, di-(C₁-C₆-alkyl)-aminothiocarbonyl, C₁-C₆-alkoxyimino-C₁-C₆-alkyl,
where the alkyl, cycloalkyl and alkoxy radicals mentioned may be partially or fully halogenated and/or may carry one to three of the following groups: cyano, hydroxyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl, or C₁-C₄-alkylcarbonyloxy; phenyl, phenyl-C₁-C₆-alkyl, phenylcarbonyl, phenylcarbonyl-C₁-C₆-alkyl, phenylsulfonylaminocarbonyl or phenyl-C₁-C₆-alkylcarbonyl,
where the phenyl radical of the 6 last-mentioned substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R⁶.

17. The compound of claim 1, wherein
A is 5- or 6-membered heteroaryl selected from the group consisting of thienyl, furyl, pyrazolyl, imidazolyl, thiazolyl, oxazolyl and pyridyl;
where the heteroaryl radicals mentioned may be partially or fully halogenated and/or may carry 1 to 3 radicals selected from the group consisting of C₁-C₆-alkyl, C₃-C₆-cycloalkyl and C₁-C₆-haloalkyl;
Het is mono- or bicyclic heteroaryl selected from the group consisting of thienyl, thiazolyl, tetrazolyl, pyridyl and indolyl,
where the heteroaryls mentioned may be partially or fully halogenated and/or may carry 1 to 2 radicals selected from the group consisting of nitro, C₁-C₄-alkyl, C₁-C₄-haloalkyl, hydroxyl, C₁-C₄-alkoxy, C₁-C₄-haloalkoxy, hydroxycarbonyl, C₁-C₄-alkoxycarbonyl, hydroxycarbonyl-C₁-C₄-alkoxy, C₁-C₄-alkoxycarbonyl-C₁-C₄-alkoxy, amino, C₁-C₄-alkylamino, di-(C₁-C₄-alkyl)amino, C₁-C₄-alkylsulfonylamino, C₁-C₄-haloalkylsulfonylamino, aminocarbonylamino, (C₁-C₄-alkylamino)carbonylamino and di-(C₁-C₄-alkyl)aminocarbonylamino;
R¹ and R² are hydrogen;
R³ is C₁-C₄-alkyl,
R⁴ is hydrogen, C₁-C₄-alkylcarbonyl, C₁-C₄-alkylaminocarbonyl, di-(C₁-C₄-alkyl)-aminocarbonyl, phenylaminocarbonyl, N—(C₁-C₄-alkyl)-N-(phenyl)aminocarbonyl, SO₂CH₃ or SO₂(C₆H₅); and
R⁵ is hydrogen.

18. The method of claim 10, wherein the heteroaroyl-substituted serineamide or salt thereof is applied at a rate of application of 0.001 to 3.0 kg/ha.

19. The method of claims 18, wherein the amount is 0.01 to 1.0 kg/ha.
